(19) United States
(12) Patent Application Publication
(10) Pub. No.: US 2024/0327549 A1
(43) Pub. Date: Oct. 3, 2024

(54) METAL-CONTAINING POLYMERS FOR MASS CYTOMETRY

(71) Applicants: THE GOVERNING COUNCIL OF THE UNIVERSITY OF TORONTO, Toronto (CA); STANDARD BIOTOOLS CANADA INC., Markham (CA)

(72) Inventors: Mitchell A. Winnik, Toronto (CA); Edmond Chi Ngae Wong, Toronto (CA); Yefeng Zhang, Markham (CA); Daniel Majonis, Markham (CA); Peng Liu, Markham (CA)

(21) Appl. No.: 18/575,268

(22) PCT Filed: Jul. 8, 2022
(86) PCT No.: PCT/CA2022/051073

§ 371 Date: Dec. 28, 2023

Related U.S. Application Data
(60) Provisional application No. 63/219,787, filed on Jul. 8, 2021.
(60) Provisional application No. 63/359,182, filed on Jul. 7, 2022.

(51) Int. Cl. C08F 8/42; C08F 20/38

(52) U.S. Cl. CPC C08F 8/42; C08F 20/38

(57) ABSTRACT

The present disclosure relates to compounds of Formula I, element tags comprising soft metals, and methods and kits therefore for performing mass cytometry.

### CROSS-REFERENCE TO RELATED APPLICATIONS

The present disclosure claims the benefit of priority from U.S. patent application No. 63/219,787, filed on Jul. 8, 2021, and from U.S. patent application No. 63/359,182, filed on Jul. 7, 2022, the contents of which are incorporated herein by reference in their entirety.

### FIELD

The present disclosure relates to metal-containing polymers and in particular soft-metal containing polymers as element tags for mass cytometry.

### INTRODUCTION

Metal-containing polymers are one of important classes of polymers developed in the 20th century.^{1,2} By incorporating different metal units into traditional organic polymers, functional polymers with novel magnetic, optic, electronic, catalytic and bioactive properties can be obtained and have found broad application in diverse fields, such as sensing, catalysis, bioimaging, drug delivery, anti-cancer drugs and biocides.³⁻⁷ Recently, a novel single-cell proteomic technique, known as mass cytometry, was developed to address the limits of multiplexing capability of conventional flow-cytometry due to spectral overlap.⁸ A feature of this technique is the use of metal-tagged antibodies in combination with inductively coupled plasma time-of-flight mass spectrometry (ICP-MS) detection. By tagging antibodies with isotopes of different heavy metal ions, one can examine the expression of multiple biomarkers in individual cells by simultaneously monitoring the signals in different mass channels. The development of mass cytometry provides new opportunities for metal-containing polymers to be used as elemental mass tags for high-parameter single-cell analysis.

Over the 15 years, several types of metal-chelating polymers have been developed with pendant aminocarboxylate chelators such as diethylenetriaminepentaaceitc acid (DTPA) or 1,4,7,10-Tetraazacyclododecane-1,4,7,10-tetraacetic acid (DOTA) for application to mass cytometry.⁹⁻¹³ These chelators are particularly effective at binding hard metal ions such as the lanthanides, yttrium and bismuth. With these polymer tags, researchers can measure more than 40 different biomarkers per cell. These chelators are less effective at binding ions of soft metals such as Pd or Pt. One study explored a series of metal-chelating polymers with a variety of aminocarboxylate pendant groups as carriers for platinum or palladium ions in mass cytometry applications.¹⁴ While the metal-containing polymers could be prepared, they were unable to stain target biomarkers and appeared to lose the heavy metal through interactions with soft ligands (likely thiols) associated with cells. However, there is a still growing demand to bring newer metals to mass cytometry and to increase the number of mass channels that can be used, and in this way to expand the capacity of mass cytometry.

### SUMMARY

Described herein are metal-chelating polymers with heterocyclic pendant groups such as dipicolylamine (DPA) and imidazole suitable for binding soft-metal ions including Re, Hg, or Ag. It has been shown that these polymers are useful in mass cytometry applications. As shown in embodiments herein, metal-tagged antibodies provide accurate quantification for single-cell immunophenotyping and can be used in conjugation with commercial reagents for mass cytometry immunoassays. A polymer comprising DPA chelating groups was employed in a 4-plex assay of PBMCs and shown to be able to quantify cell populations. DPA is an effective metal chelator for a number of different polarizable heavy metal ions. Thus, these results introduce new mass units to mass cytometry.

The resulting chelates exhibit great stability towards ligand substitution and decomposition due to the d⁶ low-spin electron configuration of Tc(I) and Re(I).^{17,19,20} It can be appreciated that the stability can be observed in other soft metals of similar electron configuration. Since each soft metal element has multiple naturally occurring isotopes, soft-metal chelating polymers enables new mass channels for mass cytometry applications.

Metal-containing polymers to be employed in mass cytometry applications may have one or more of the following characteristics: First, the polymer may have a relatively narrow distribution of chain lengths such that each labeled antibody carries a similar number of metal ions. Second, the metals can be bound in a way that they do not undergo little or no exchange during storage of applications (e.g., in lyophilized form) and/or during use in aqueous solution for the hours over which a mass cytometry experiment may take place. Third, the polymer may contain functional groups for antibody conjugation. Finally, the polymer can be water-soluble since bioassays are performed in aqueous media. Simultaneously satisfying multiple characteristics above represents a synthetic challenge.

In one aspect, the present disclosure includes A compound of Formula I

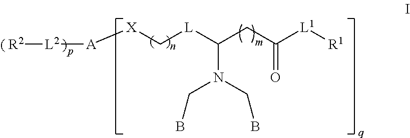

wherein
A is a polymer backbone, optionally, the polymer is a linear polymer, branched polymer, hyperbranched polymer, co-polymer, or combinations thereof;
each B is independently a nitrogen-containing 5-membered to 7-membered heterocycle, optionally substituted with one or more polar functional groups selected from C1-C6 COOH, C1-C6 alkoxy, C1-C6 alkyl phosphonate, alkyl ether, polyether, or combinations thereof;
L is absent or a linker;
each L² is independently absent or a linker;
each R² is independently a first modifying group selected from a solubility modifier, a reactive functional group, a biomolecule, or combinations thereof;
X is a functional group selected from ester, ether and amide;
each L¹ is independently absent or a linker;
each R¹ is independently H, C1 to C8 alkyl, C3-C8 cycloalkyl, OH, C1 to C10 alkoxy, C1 to C10 alkyl amine, a solubility modifier, a reactive functional group, a biomolecule and combinations thereof;
n is an integer between 0 to 50;
m is an integer between 0 to 40;
p is an integer between 0 to 30; and
q is an integer above 0.

In another aspect, the present disclosure includes a compound of Formula I, wherein the compound of Formula I is chelated to one or more metal M, and wherein the compound has a structure of Formula II

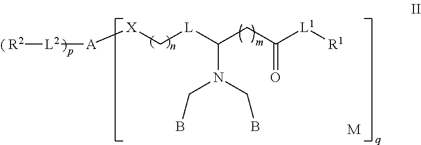

or a derivative or salt thereof.

In another aspect, the present disclosure includes a composition comprising one or more compounds of Formula I and one or more metals M.

In another aspect, the present disclosure includes a compound of Formula I or II for use in mass cytometry.

In another aspect, the present disclosure includes an element tag comprising a linear or a branched polymer comprising a plurality of chelating groups, wherein at least one chelating group is chelated to a soft metal atom of the soft metal, the soft metal being a single isotope.

In another aspect, the present disclosure includes a kit comprising
an isotopic composition comprising multiple soft metal atoms of a single isotope of a soft metal; and
an element tag comprising a linear or branched polymer comprising a plurality of chelating groups comprising two nitrogen-containing 5-membered or 6-membered heterocycles, wherein each chelating group includes at least one soft metal atom of the isotopic composition or is capable of binding at least one soft metal atom of the isotopic composition;
wherein the kit does not comprise any radioactive soft metal.

In another aspect, the present disclosure includes a method comprising:
providing an isotopic composition comprising multiple soft metal atoms of a single isotope of a soft metal;
providing an element tag comprising a linear or branched polymer comprising a plurality of chelating groups each independently comprising two nitrogen-containing 5-membered or or 6-membered heterocycles, wherein each chelating group is capable of binding at least one of the soft metal atoms of the isotopic composition; and
binding the soft metal atoms of the isotopic composition to the one or more chelating groups of the element tag;
wherein the soft metal atoms are non-radioactive.

In another aspect, the present disclosure includes a method for the analysis of an analyte in a biological sample, comprising:
(i) incubating an element tagged affinity reagent with the analyte, the element tagged affinity reagent comprising an affinity reagent tagged with an element tag, the element tag comprising a linear or branched polymer having multiple chelating groups each independently comprising two nitrogen-containing 5-membered or 6-membered heterocycles, the element tag further comprising multiple soft metal atoms of a single isotope of a soft metal;
wherein:
   each chelating group of the element tag includes at least one of the soft metal atoms or is capable of binding at least one of the soft metal atoms,
   the soft metal atoms are non-radioactive, and
   the affinity reagent specifically binds the analyte,
(ii) separating unbound element tagged affinity reagent from bound element tagged affinity reagent; and
(iii) analyzing the element tag bound to the affinity reagent attached to the analyte by mass spectrometric atomic spectroscopy.

### DRAWINGS

Illustrative embodiments of the present disclosure will be further described in relation to the drawings in which:

FIG. 1 is a ¹H-NMR (600 MHz) spectrum of compound I-1.

FIG. 2(a) is a ¹H-NMR (600 MHz) spectrum of the aromatic region of the Re-loaded polymer. FIG. 2(b) is a FTIR spectra of the Re salt, compound I-1, and Re-loaded compound II-1.

FIG. 3(a)-(e) are biaxial scatter plots of ¹⁷⁰Er-CD3 vs. ¹⁸⁷Re-CD20 within human PBMCs at different titers. FIG. 3(f) is a biaxial scatter plot of ¹⁷⁰Er-CD3 vs. ¹⁴⁷Sm-CD20 within human PMBCs at optimal titer.

FIG. 4(a) is a ¹H-NMR (600 MHz) spectrum of RAFT reaction mixture and FIG. 4(b) is a GPC trace of poly(PFPA) synthesized by RAFT polymerization of PFPA monomer.

FIG. 5 is a ¹H-NMR (600 MHz) spectrum of poly(PFPA) and a ¹⁹F-NMR (564 MHz) spectrum of poly(PFPA).

FIG. 6 is a ¹H-NMR (600 MHz) spectrum of compound I-2 (top), compound I-3 (middle), and compound I-4 (bottom).

FIG. 7 is a series ¹⁹F-NMR (564 MHz) spectra depicting aminolysis of PolyPFPA with lysine-based rhenium chelator.

FIG. 8 is a ¹H-NMR (600 MHz) spectrum of polymer 2-2 and a ¹⁹F-NMR (564 MHz) spectrum of polymer 2-2.

FIG. 9 is a UV-vis spectrum of polymer 2-2 and DDMAT CTA.

FIG. 10 is a ¹H-NMR (600 MHz) spectrum of polymer 2-3 by PEGlylation of polymer 2-2.

FIG. 11(a) is a ¹H-NMR (600 MHz) spectrum of Bis-Mal-PEG₆. FIG. 11(b) is a ¹H-NMR (600 MHz) spectrum of Bis-Mal-PEG₆ and rhenium salt mixture.

FIG. 12 is an image of lyophilized rhenium-loaded polymer compound II-1.

FIG. 13(a) is a UV-vis spectrum of Re-loaded polymer compound II-1 in PBS. FIG. 13(b) is a FPLC chromatogram of pure CD20 antibody. FIG. 13(c) is a FPLC chromatogram of antibody-polymer conjugate.

FIG. 14(a) is a ¹H-NMR (600 MHz) spectrum of Polymer 3-2/I-5 and FIG. 14(b) is a ¹H-NMR (600 MHz) spectrum of compound I-1.

FIG. 15 is a FTIR spectrum of Polymer 3-2/I-5.

FIG. 16 is a ¹H-NMR (600 MHz) spectrum of Pt-loaded Polymer 4-1/II-2.

FIG. 17 is a is a series of biaxial scatter plots of ¹⁷⁰Er-CD3 vs. ¹⁹⁵Pt-CD20 within human PBMCs at different titers and ¹⁷⁰Er-CD3 vs. ¹⁴⁷Sm-CD20 within T lymphocytes and B lymphocytes.

FIG. 18 is a ¹H-NMR (600 MHz) spectrum of Polymer 2-3.

FIG. 19 is a ¹H-NMR (600 MHz) spectrum of Hg-loaded Polymer 5-1/II-3.

FIG. 20 is a ¹H-NMR (600 MHz) spectrum of Ag-loaded Polymer 6-1/II-4.

FIG. 21 is a ¹H-NMR (600 MHz) spectrum of (a) Pt-loaded Polymer 14-3/II-6, where the arrow shows chemical shift change of the pyridyl protons after metalation with Pt, and (b) Hg-loaded Polymer 14-2/II-5, where the arrow shows chemical shift change of the pyridyl protons after metalation with Hg.

FIG. 22 is a ¹H-NMR (600 MHz) spectrum of the compound 11-4.

FIG. 23 is a ¹H-NMR (600 MHz) spectrum of the compound I-11.

FIG. 24 is a ¹H-NMR (600 MHz) spectrum of the compound I-12.

FIG. 25 is the mass cytometry immunoassay results of identification of CD20+ B cells from PBMCs by rhenium-tagged zwitterionic solubility modifier containing polymers of the present disclosure conjugated to CD20 antibodies at various concentrations of the polymer conjugate. Maxpar™ ¹⁴⁷Sm-CD20 conjugate was used as a positive control.

FIG. 26 is the mass cytometry immunoassay results of identification of CD8+ T cells from PBMCs by rhenium-tagged zwitterionic solubility modifier containing polymers of the present disclosure conjugated to CD8a antibodies at various concentrations of the polymer conjugate. Maxpar™ ¹⁴⁶Nd-CD8a conjugate was used as a positive control.

FIG. 27 is a graph showing signal distribution histograms of ¹⁸⁷Re signals and ¹⁴⁷Sm signals obtained from non-T/B cells (CD3-CD20−) within PBMCs with rhenium-tagged zwitterionic solubility modifier containing polymers of the present disclosure conjugated to CD20 antibodies at various concentrations of the polymer conjugate. Maxpar™ ¹⁴⁷Sm-CD20 conjugate was used as a control.

FIG. 28 is a graph showing signal distribution histograms of ¹⁸⁷Re signals and ¹⁴⁶Nd signals obtained from B cells (CD3-CD20+) within PBMCs with rhenium-tagged zwitterionic solubility modifier containing polymers of the present disclosure conjugated to CD8a antibodies at various concentrations of the polymer conjugate. Maxpar™ ¹⁴⁶Nd-CD8a conjugate was used as a control.

FIG. 29 is a graph showing results from the non-specific binding tests of both PEG modified rhenium polymer (group A, polymer concentration of 1, 2 and 5 ug/mL) and zwitterion modified rhenium polymer (group B, polymer concentration of 1, 2 and 5 ug/mL).

FIG. 30 is a ¹H-NMR (600 MHz) spectrum of the compound 16-3.

FIG. 31 is a series of graphs showing results from non-specific binding tests of glutathione modified polymers of the present disclosure vs non-glutathione modified polymers of the present disclosure. Maxpar™ was used as positive control.

### DESCRIPTION OF VARIOUS EMBODIMENTS

### 1. Definitions

Unless otherwise indicated, the definitions and embodiments described in this and other sections are intended to be applicable to all embodiments and aspects of the present disclosure herein described for which they are suitable as would be understood by a person skilled in the art.

The term “compound of the disclosure” or “compound of the present disclosure” and the like as used herein refers to a compound of Formula I or II, and salts, solvates and/or derivatives thereof.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the disclosure exist as individual salts and hydrates, as well as a combination of, for example, a solvate of a salt of a compound of the disclosure.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. For example, a metal chelated to a second component can be different from a metal chelated to a first component, when the second component and the first component can have the same chelator. A “third” component is different from the other, first, and second components, and further enumerated or “additional” components are similarly different.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or condition would depend on the specific synthetic manipulation to be performed, the identity of the molecule(s) to be transformed and/or the specific use for the compound, but the selection would be well within the skill of a person trained in the art.

In embodiments of the present disclosure, the compounds described herein may have at least one asymmetric center. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (for example, less than 20%, suitably less than 10%, more suitably less than 5%) of compounds of the present disclosure having an alternate stereochemistry. It is intended that any optical isomers, as separated, pure or partially purified optical isomers or racemic mixtures thereof are included within the scope of the present disclosure.

The compounds of the present disclosure may also exist in different tautomeric forms and it is intended that any tautomeric forms which the compounds form, as well as mixtures thereof, are included within the scope of the present disclosure.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least or up to ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cn1-n2”. For example, the term C1-10alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cn1-n2”. For example, the term C2-6alkylene means an alkylene group having 2, 3, 4, 5 or 6 carbon atoms.

The term “available”, as in “available hydrogen atoms” or “available atoms” refers to atoms that would be known to a person skilled in the art to be capable of replacement by a substituent.

The term “amine” or “amino,” as used herein, whether it is used alone or as part of another group, refers to groups of the general formula NR′R″, wherein R′ and R″ are each independently selected from hydrogen or C1-6alkyl.

The term “cycloalkyl,” as used herein, whether it is used alone or as part of another group, means a saturated carbocyclic group containing one or more rings. The number of carbon atoms that are possible in the referenced cycloalkyl group are indicated by the numerical prefix “Cn1-n2”. For example, the term C3-10cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring. In an embodiment of the disclosure, the aryl group contains from 6, 9 or 10 carbon atoms, such as phenyl, indanyl or naphthyl.

The term “heterocycle”, “heterocyclic” and the like as used herein, whether it is used alone or as part of another group, refers to cyclic groups containing at least one aromatic or non-aromatic ring in which one or more of the atoms are a heteroatom selected from O, S and N. Heterocyclic groups are either saturated or unsaturated (i.e. contain one or more double bonds). When a heterocyclic group contains the prefix Cn1-n2 this prefix indicates the number of carbon atoms in the corresponding carbocyclic group, in which one or more, suitably 1 to 5, of the ring atoms is replaced with a heteroatom as defined above.

The term “heteroaryl”, “heteroaromatic”, and the like, as used herein, whether it is used alone or as part of another group, refers to cyclic groups containing at least one heteroaromatic ring in which one or more of the atoms are a heteroatom selected from O, S and N. When a heteroaryl group contains the prefix Cn1-n2 this prefix indicates the number of carbon atoms in the corresponding carbocyclic group, in which one or more, suitably 1 to 5, of the ring atoms is replaced with a heteroatom as defined above.

All cyclic groups, including aryl and cyclo groups, contain one or more than one ring (i.e. are polycyclic). When a cyclic group contains more than one ring, the rings may be fused, bridged, spirofused or linked by a bond.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halo” as used herein refers to a halogen atom and includes fluoro, chloro, bromo and iodo.

The term “optionally substituted” refers to groups, structures, or molecules that are either unsubstituted or are substituted with one or more substituents.

The term “atm” as used herein refers to atmosphere.

The term “MS” as used herein refers to mass spectrometry.

The term “aq.” as used herein refers to aqueous.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3rd Edition, 1999 and in Kocienski, P. Protecting Groups, 3rd Edition, 2003, Georg Thieme Verlag (The Americas).

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “cell” as used herein refers to a single cell or a plurality of cells and includes a cell either in a cell culture or optionally in a subject.

The term “solvate” as used herein means a compound, or a salt or derivative of a compound, wherein molecules of a suitable solvent are incorporated in the crystal lattice. Examples of suitable solvents can include ethanol, water and the like. When water is the solvent, the molecule is referred to as a “hydrate”.

The term “antibody” as used herein is intended to include any and all antibodies and fragments thereof, including monoclonal antibodies, polyclonal antibodies, and chimeric antibodies and binding fragments thereof. The antibody may be from recombinant sources and/or produced in transgenic animals. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. Antibody fragments as used herein mean binding fragments

The term “oligonucleotide” as used herein refers to a nucleic acid comprising, a sequence of nucleotide or nucleoside monomers consisting of naturally and non-naturally occurring bases, sugars, and intersugar (backbone) linkages, and includes single-stranded and double-stranded molecules, RNA and DNA. Oligonucleotides may be long (e.g. greater than 1000 monomers and up to 10K monomers), medium sized (e.g. between and inclusive of 200 and 1000 nucleotides) or short for example less than 200 monomers, 100 monomers, 50 monomers, including non-naturally occurring monomers. The term “oligonucleotide” includes, for example, single stranded DNA (ssDNA), genomic DNA (gDNA), complementary DNA (cDNA, reverse transcribed from an RNA), messenger RNA (mRNA), “antisense oligonucleotides” and “miRNA” as well as oligonucleotide analogues such as “morpholino oligonucleotides”, “phosphorothioate oligonucleotides”, or any oligonucleotide or analog thereof known to one of skill in the art.

The term “element tag”, “tag” and the like as used herein refers to a chemical moiety which includes an element or multitude of elements having one or many isotopes (such as soft metals) attached to a supporting molecular structure, or that is capable of binding said element(s) or isotope(s). The element tag can also comprise the means of attaching the element tag to a molecule of interest or target molecule (for example, a biomolecule such as an analyte). Different element tags may be distinguished on the basis of the elemental composition of the tags. An element tag can contain many copies of a given isotope and can have a reproducible copy number of each isotope in each tag. An element tag is functionally distinguishable from a multitude of other element tags in the same sample because its elemental or isotopic composition is different from that of the other tags.

The term “ICP-MS” as used herein refers to the Inductively Coupled Plasma Mass Spectrometer—a sensitive mass spectrometry based elemental analyzer. Different ICP-MS configurations are primarily distinguished by the mass selecting technique employed and can be, for example the quadrupole or time-of-flight (ICP-TOF) or magnetic sector (high resolution ICP-MS). There are many commercially available ICP-MS models having a wide spectrum of configurations, capabilities and modifications.

The term “polymer” as used herein refers to a substance composed of molecules characterized by the multiple repetitions of one or more species of atoms or groups of atoms (constitutional units) linked to each other in amounts sufficient to provide a set of properties that do not vary markedly with the addition or removal of one or a few constitutional units. (IUPAC definition, see E. S. White, J. Chem. Inf. Comput. Sci. 1997, 37, 171-192). A polymer molecule can be thought of in terms of its backbone, the connected link of atoms that span the length of the molecule, and the pendant groups, attached to the backbone portion of each constituent unit. The pendant groups are often chemically and functionally different from the backbone chain. Pendant groups that have a high affinity for metal ions can act as chelating groups or ligands for those ions. In some instances, a polymer can have about 10 to about 300 units.

The term “copolymers” as used herein refers to polymers that consist of two or more chemically different constituent units. A “linear polymer” is a polymer characterized by a linear sequence of constituent units. A “block copolymer” is a linear polymer with sequences of constituent units of a common type, joined to sequences of constituent units of a different type. A “branched polymer” is a polymer in which additional polymer chains (the branches) issue from the backbone of the polymer. One commonly refers to the longest linear sequence as the “main chain”. A branched polymer in which the chemical composition of the constituent units of the branch chains is different than those of the main chain is called a “graft copolymer”.

The term “star polymers” as used herein refers to polymers that have multiple linear polymer chains emanating from a common constituent unit or core. The term “hyperbranched polymers” as used herein refers to multiple branched polymers in which the backbone atoms are arranged in the shape of a tree. These polymers are related to “dendrimers”, which have three distinguishing architectural features: an initiator core, interior layers (generations) composed of repeating units radially attached to the initiator core, and an exterior surface of terminal functionality attached to the outermost generation. “Dendrimers” differ from hyperbranched polymers by their extraordinary symmetry, high branching, and maximized (telechelic) terminal functionality.

The term “metal tagged polymer” (also a “polymeric metal tag carrier”, or “metal-polymer conjugate”, or “chelate-derivatized polymer”) and the like as used herein refers to a variety of the element tag which consists of a polymer backbone bearing at least one pendant chelating group with metal atoms attached to them. These metal-tagged polymers can be, but are not limited to, linear, star, branched, or hyperbranched homopolymers or copolymers as well as block or graft copolymers.

The term “metal binding pendant group” as used herein is a pendant group on the polymer that is capable of binding a metal or an isotope of a metal. It can also be referred to as a chelator.

The term “chelation” as used herein refers to the process of binding of a ligand, the chelant, chelator or chelating agent, to a metal ion, forming a metal complex, the chelate. In contrast to the simple monodentate ligands like H₂O or NH₃, the polydentate chelators form multiple bonds with the metal ion.

The term “metal” as used herein refers to an element having one of the following atomic numbers 3, 4, 11-13, 19-33, 37-52, 55-84, 87-102.

The term “soft metal” as used herein refers to a metal that is considered soft according to the Pearson's Hard and Soft Lewis Acids and Bases theory.

It is envisioned that when referring to a single isotope, it is referring to substantially a single isotope of a metal. For example, a single isotope can contain trace amounts of other isotopes of the metal and/or trace amounts of another metal. For example, substantially a single isotope can mean an isotope having a purity of the isotope of any one of at least 95%, at least 96%, at least 97%, at least 98%, at least 99%, at least 99.5%, or at least 99.9%, or having a purity of 100% of the isotope. For instance, a single isotope can comprise about 95% or above 95% of the isotope and about 5% or less than 5% of other isotopes. In some embodiments, a single isotope can comprise about 97% or above 97% of the isotope and about 3% or less than 3% of other isotopes. In some embodiments, a single isotope can comprise about 98% or above 98% of the isotope and about 2% or less than 2% of other isotopes. In some embodiments, a single isotope can comprise about 99% or above 99% of the isotope and about 1% or less than 1% of other isotopes. In some embodiments, a single isotope can comprise about 99.5% or above 99.5% of the isotope and about 0.5% or less than 0.5% of other isotopes. In some embodiments, a single isotope can comprise about 99.9% or above 99.9% of the isotope and about 0.1% or less than 0.1% of other isotopes. In some embodiments, a single isotope comprises 100% of the isotope.

The terms Mn, Mw and PDI (polydispersity index): Mw/Mn are used to indicate the number and average molecular weight and the polydispersity index describes the molecular weight distribution, respectively.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

For ranges described herein, subranges are also contemplated, for example every, 0.1 increment there between. For example, if the range is 0 ppm to about 5 ppm, also contemplated are 0.1 ppm to about 5 ppm, 0 ppm to about 4.9 ppm, 0.1 ppm to about 4.9 ppm and the like.

### II. Compounds, Compositions and Kits

In one aspect, the present disclosure includes A compound of Formula I

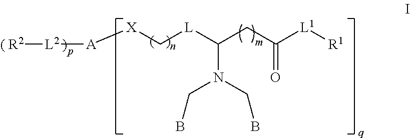

wherein
   A is a polymer backbone, optionally, the polymer is a linear polymer, branched polymer, hyperbranched polymer, co-polymer, or combinations thereof;
   each B is independently a nitrogen-containing 5-membered to 7-membered heterocycle, optionally substituted with one or more polar functional groups selected from C1-C6 COOH, C1-C6 alkoxy, C1-C6 alkyl phosphonate, alkyl ether, polyether, or combinations thereof;
   L is absent or a linker;
   each L² is independently absent or a linker;
   each R² is independently a first modifying group selected from a solubility modifier, a reactive functional group, a biomolecule, or combinations thereof;
   X is a functional group selected from ester, ether and amide;
   each L¹ is independently absent or a linker;
   each R¹ is independently H, C1 to C8 alkyl, C1 to C8 alkyl, C2 to C8 alkenyl, C3-C8 cycloalkyl, OH, C1 to C10 alkoxy, C1 to C10 alkyl amine, a solubility modifier, a reactive functional group, a biomolecule and combinations thereof;
   n is an integer between 0 to 50;
   m is an integer between 0 to 40;
   p is an integer between 0 to 30; and
   q is an integer above 0.

In another aspect, the present disclosure includes a compound of Formula I, wherein the compound of Formula I is chelated to one or more metal M, and wherein the compound has a structure of Formula II

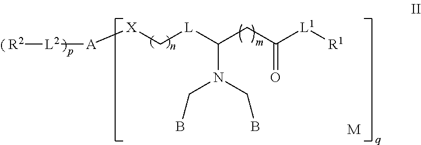

or a derivative or salt thereof.

In another aspect, the present disclosure includes a composition comprising one or more compounds of Formula I or one or more compounds of Formula II, and a solvent.

In another aspect, the present disclosure includes a compound of Formula I or II for use in mass cytometry.

In some embodiments, the polymeric backbone A can include from 10 to 300 monomeric units.

In some embodiments, n is an integer between 0 to 20, between 1 and 10, or between 0 and 7. In some embodiments, m is an integer between 0 to 30, between 0 and 20, between 0 and 10, or between 0 and 4. In some embodiments, p is an integer between 0 and 20, between 0 and 10, between 0 and 5 or between 0 to 3. In some embodiments, q is an integer above 0. For example, q is an integer between 2 to 300, between 2 to 200, between 2 to 150, between 2 and 100, between 4 and 80, between 4 and 60, between 4 and 20, between 4 and 12, or between 10 and 60. For example, q is an integer that is at least 2, at least 4, or at least 10. For example, q is an integer that is up to 300, up to 250, up to 200, up to 150, up to 100, up to 80, up to 60, up to 20, up to 12, or up to 10. In some embodiments, q may be greater than 1 but less than 20 to avoid steric hindrance and/or reduce background, such as when used for staining tissue for imaging mass cytometry or for labeling intracellular targets in suspension mass cytometry. In some embodiments, n is an integer between 0 to 7; m is an integer between 0 to 4; p is an integer between 0 to 3; and q is an integer above 0. For example, n is 2, 3, 4, or 5. In some embodiments, m is 0, 1, or 2. In some embodiments, p is 1 or 2.

As used herein, the term “modifying group” such as in a first modifying or a second modifying group refers to a group, a moiety, a structure, and/or a substituent that when attached to a chemical entity or chemical structure such as a polymer modifies, changes, adjusts, or alters the functionality and/or properties of the chemical entity or chemical structure such as the polymer. For example, a modifying group can modify, change, adjust or alter the solubility, reactivity, and/or hydrophobicity of a chemical entity, or the affinity of the chemical entity towards another chemical entity. In some embodiments, a modifying group can be a solubility modifier, and/or a reactive functional group.

As used herein, a “solubility modifier” refers to a group, a moiety, a structure and/or a substituent that when attached to a chemical entity or chemical structure such as an oligomer or a polymer modifies, changes, adjusts, or alters the solubility of the chemical entity or chemical structure in water. For example, a solubility modifier can include a water soluble polymer such as polyethyleneglycol (PEG), a zwitterionic polymer, or a charged polymer. The zwitterionic polymer can include poly(sulfobetaine methacrylate) (PSBMA) and poly(carboxybetaine methacrylate) (PCBMA). In some embodiments, the solubility modifier of the first modifying group of each R² and the solubility modifier of the second modifying group each independently comprises polyethylglycol (PEG), sugar, oligosaccharide, or zwitterionic polymer such as poly(carboxylbetaine) methacrylate or poly(sulfobetaine) methacrylate (PBSMA). The solubility modifier may increase the solubility of the polymer (e.g., polymer loaded with a metal as described herein) compared to if the solubility modifier were absent, such as a two-fold increase in the amount of the polymer that can be in a solution (e.g. an aqueous solution, such as a solution buffered at a pH at or between 6 and 8). In certain aspects, the solubility modifier may increase the solubility of the polymer (e.g., polymer loaded with a metal as described herein) compared to if the solubility modifier were absent. In some embodiments, the oligomer can have up to 10 monomeric units. In some embodiments, the solubility modifier can include a polymer that has from about 10 to about 5000 units. For example, the solubility modifier can be a PEG group. For example, the PEG group can have about 10 to about 350 units, about 10 to about 300 units, about 10 to about 250 units, about 10 to 200 units, about 10 to 150 units, or about 110 units of ethylene glycol. For example, the PEG group can have at least 10, at least 20, or at least 30 units of ethylene glycol. For example, the PEG group can have up to 300, up to 250, up to 200, up to 150, up to 100, or up to 50 units of ethylene glycol. For example, the PEG group can have a Mn of about 5000 g/mol to about 10000 g/mol.

In certain aspects, the solubility modifier can also reduce non-specific binding of the compounds of the present disclosure to a target in a sample. In some embodiments, it has been shown that certain solubility modifiers are more effective at reducing non-specific binding. It has been shown for example, that a zwitterionic solubility modifier demonstrated less non-specific binding than a PEG solubility modifier.

In some embodiments, at least a portion or all of the metal M can be coordinated to one or more solubility modifiers. For example, the solubility modifier can be a ligand of the metal M. In some embodiments, the solubility modifier can comprise a thiol small molecule. For instance, the thiol small molecule can be selected from glutathione, cysteine, thioglycolic acid, mercaptosuccinic acid, methyl thioglycolate, dimercaprol, dimercaptosuccinic acid, 2,3-dimercapto-1-propanesulfonate, and combinations thereof. In some embodiments, the solubility modifier is glutathione. It can be appreciated that the solubility modifier can be coordinated to the metal M by ligand exchange reactions.

As used herein, a “reactive functional group” refers to a group of atoms or a single atom that interacts or reacts with another group of atoms or a single atom to form a chemical interaction between the two groups of atoms or the two atoms. For instance, attaching one or more reactive functional groups on a chemical entity or chemical structure such as a polymer modifies or changes the reactivity of the chemical entity or chemical structure such as the polymer to allow the chemical entity or chemical structure to interact or react with groups of atoms on another chemical entity or chemical structure such as a biomolecule. It can be appreciated that in some instances, a given reactive functional group can interact or react with a specified functional group to form a chemical interaction. For example, it is known that azide is amenable to click chemistry and that maleimide can react with thiol. In some embodiments, the chemical interaction is covalent or ionic. For example, the chemical interaction is covalent. In some embodiments, the reactive functional group is for attachment to one or more biomolecules. In some embodiments, the reactive functional group of the first modifying group of each R² and the reactive functional group of the second modifying group is each independently selected from carboxylic acid, N-hydroxysuccinimide ester, tetrafluorophenyl ester, pentafluorophenyl ester, maleimide, thiol, azide, dibenzocyclooctyne (DBCO), trans-cyclooctene (TCO), tetrazine, furan, hydrazide, or aldehyde.

It can be appreciated that the reactive functional group can be reversibly protected or capped with suitable protective groups until the reactive functional group is needed for further reaction. For example, a thiol can be capped with a thiol capping group such as maleimide or other groups known in the art. For example, a thiol containing polymer can be temporarily protected or can temporarily exist as a disulfide dimer, which can be reduced using known methods (e.g. DTT reduction) to reveal the thiol group as needed. Thus, it is contemplated that the reactive functional group as described herein also includes protected versions of the reactive functional group.

As used herein, when a metal is non-radioactive, it means that the metal is essentially non-radioactive. For example, a radioactive metal can have a decay rate that is suitable for use in radiometric detection assays, whereas a non-radioactive metal can have a decay rate that is not suitable for radiometric detection assays, or below detection limit of common radiometric detection assays used in the field of radiolabelling. In some embodiments, non-radioactive metals can have a half-life of more than about 150,000 years, more than 200,000 years, or more than about 210,000 years. For example, it is known that ⁹⁹Tc isotope has a half-life of 210,000 years and is thus considered non-radioactive for the purpose of the present disclosure.

In some embodiments, each B is independently a 5- or 6-membered heterocycle. For example, each B can be independently substituted or unsubstituted tetrahydropyrrole. In some embodiments, each B is independently a nitrogen-containing 5-membered or 6-membered heteroaryl, optionally substituted with one or more polar functional groups selected from COOH, C1-C6 alkoxy, C1-C6 alkyl phosphonate, alkyl ether, polyether, or combinations thereof, and wherein optionally one or more B are coordinated to a soft metal, and/or conjugated to one or more biomolecules.

For example, each B is independently pyridine or imidazole, optionally substituted with one or more polar functional groups selected from C1 to C5 alkyl, C2 to C5 alkenyl, COOH, C1-C6 alkoxy, C1-C6 alkyl phosphonate, alkyl ether, polyether, or combinations thereof, and wherein optionally one or more B are coordinated to a soft metal, and/or conjugated to one or more biomolecules.

It is contemplated that two B attached to the same nitrogen are not necessarily the same chelating group. Nevertheless, out of synthetic ease, the two B attached to the same nitrogen can be the same.

In some embodiments, one or more B are coordinated to a soft metal. It is contemplated that it is not necessary that all B of a compound of Formula I are coordinated to a metal. For example, in some embodiments, about 30% to about 95%, about 40% to about 90%, about 50% to about 85% of B are coordinated to a metal. In some embodiments, at least or about 50%, at least or about 55%, at least or about 60%, at least or about 65%, at least or about 70%, at least or about 75%, at least or about 80%, or at least or about 85% of B are coordinated to a metal. In some embodiments, up to 95%, up to 90%, up to 85%. Up to 80%, up to 75%, up to 70%, or up to 65% of B are coordinated to a metal. In some embodiments, about 75% to about 80% of B are coordinated to a metal. In some embodiments, all of B are coordinated to a metal. Without wishing to be bound by theory, it can be appreciated that two B attached to the same nitrogen atom can chelate to a same metal atom in a bidentate fashion.

In some embodiments, B is optionally substituted pyridine.

In some embodiments, B is substituted or unsubstituted imidazole. Optionally, B is 2-substituted or 4-substituted imidazole. Suitable imidazole-based chelators include those described in Maresca et al., Bioconjugate Chem., 2010, 21, 1032, the content of which is incorporated in its entirety by reference.

In some embodiments, R¹ is a biomolecule. For example, R¹ can be an antibody. In some embodiments, R¹ is an affinity reagent.

In some embodiments, R² is a biomolecule. For example, R² can be an antibody. In some embodiments, R² is an affinity reagent.

In some embodiments, X is amide. For example, X is —C(O)NR⁴— or —NR⁴C(O)—, wherein R⁴ is H or C1 to C4 alkyl.

In some embodiments, X is —C(O)NR⁴— and the compound has a structure of Formula Ia

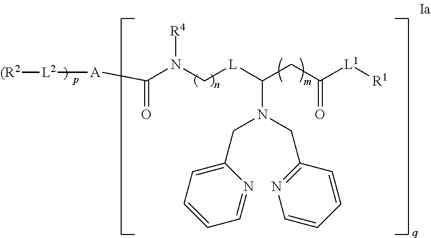

In some embodiments, X is —NR⁴C(O)— and the compound has a structure of Formula Ib

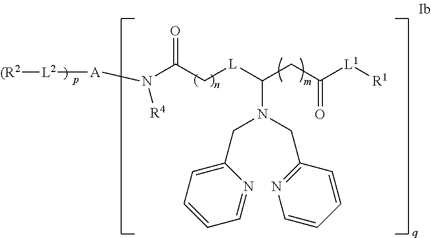

In some embodiments, X is —C(O)NR⁴— and the compound has a structure of Formula Ic

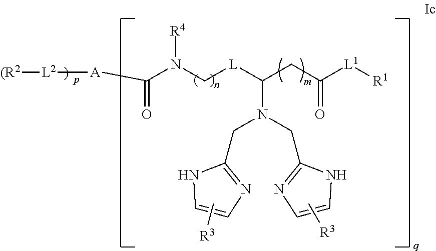

wherein each R³ is independently selected from H, C1 to C5 alkyl, C2 to C5 alkenyl, C1-C6 COOH, C1-C6 alkoxy, C1-C6 alkyl phosphonate, alkyl ether, or polyether.

In some embodiments, X is —C(O)NR⁴— and the compound has a structure of Formula Id or Ie

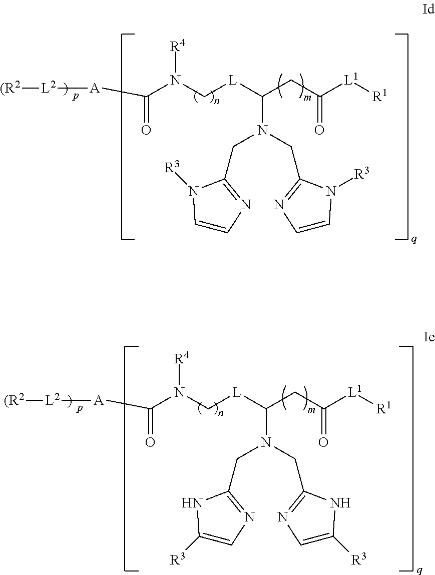

In some embodiments, X is —NR⁴C(O)— and the compound has a structure of Formula If

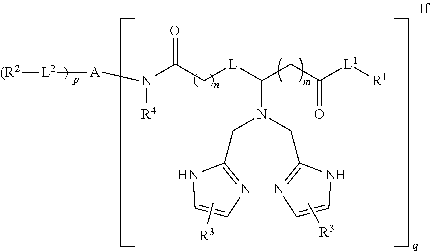

wherein each R³ is independently selected from H, C1 to C5 alkyl, C2 to C5 alkenyl, C1-C6 COOH, C1-C6 alkoxy, C1-C6 alkyl phosphonate, alkyl ether, or polyether.

In some embodiments, X is —NR⁴C(O)— and the compound has a structure of Formula Ig or

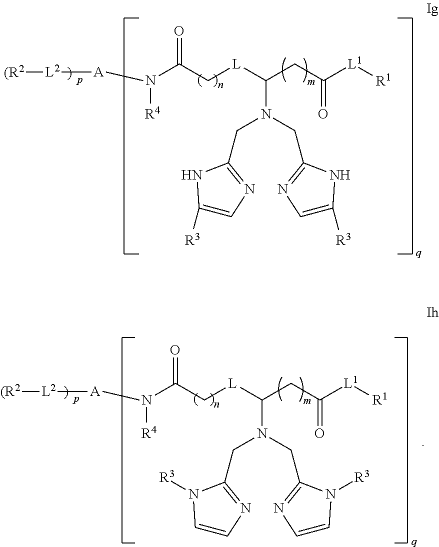

In some embodiments, R⁴ is H. In some embodiments, R⁴ is C1 to C3 alkyl.

In some embodiments, the compound of Formula I has a structure of Formula II

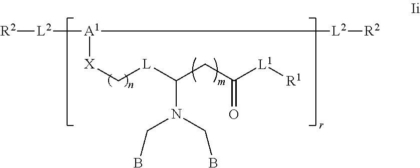

wherein A¹ is a monomer of A, and r is about 3 to about 300, about 3 to about 250, about 3 to about 200, about 3 to about 150, about 3 to about 100, about 3 to about 50, about 6 to about 30, or about 10 to about 25. In some embodiments, r is up to 300, up to 250, up to 200, up to 150, up to 100, up to 50, up to 30, or up to 25. In some embodiments, r is at least 3, at least 6, or at least 10. Optionally, the polymer backbone A is a linear polymer or copolymer.

In some embodiments, the compound of Formula I has a structure of Formula Ij

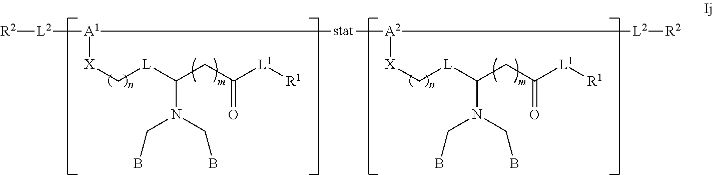

wherein A¹ and A² are each a monomer of A, and r is about 3 to about 300, about 3 to about 250, about 3 to about 200, about 3 to about 150, about 3 to about 100, about 3 to about 50, about 6 to about 30, or about 10 to about 25, and the polymer backbone A is a linear copolymer copolymer. In some embodiments, r is up to 300, up to 250, up to 200, up to 150, up to 100, up to 50, up to 30, or up to 25. In some embodiments, r is at least 3, at least 6, or at least 10.

In some embodiments, each R³ is independently selected from H, —(CH₂)₁₋₃COOH, —(CH₂)₁₋₃O(CH₂)₁₋₂CH₃, —(CH₂)₂₋₄OH, —(CH₂)₂₋₅P(O)(OCH₂CH₃)₂, or —CH₂CH(OMe)₂.

In some embodiments, A is selected from polyacrylate, polyacrylamide, polyether, polyamino acid, polyvinyl amine, poly(2-oxazoline), polyethylene glycol, polysaccharide, dendrimer, co-polymers thereof, or combinations thereof.

For example, A can be a polyamino acid. For instance, the polyamino acid can be optionally substituted polyglutamic acid, polyaspartic acid, polylysine, poly(2,4-dimethylaminobutyric acid) (polyDab), poly(2,4-diaminopimelic acid) (polyDap), derivatives thereof, or combinations thereof.

It is contemplated that the polymer backbone A of compound of the present disclosure can be a copolymer. For example, it can be a copolymer comprising PEG.

In some embodiments, the polymer backbone is a linear polymer. For example, the compound of Formula I can have a structure shown below.

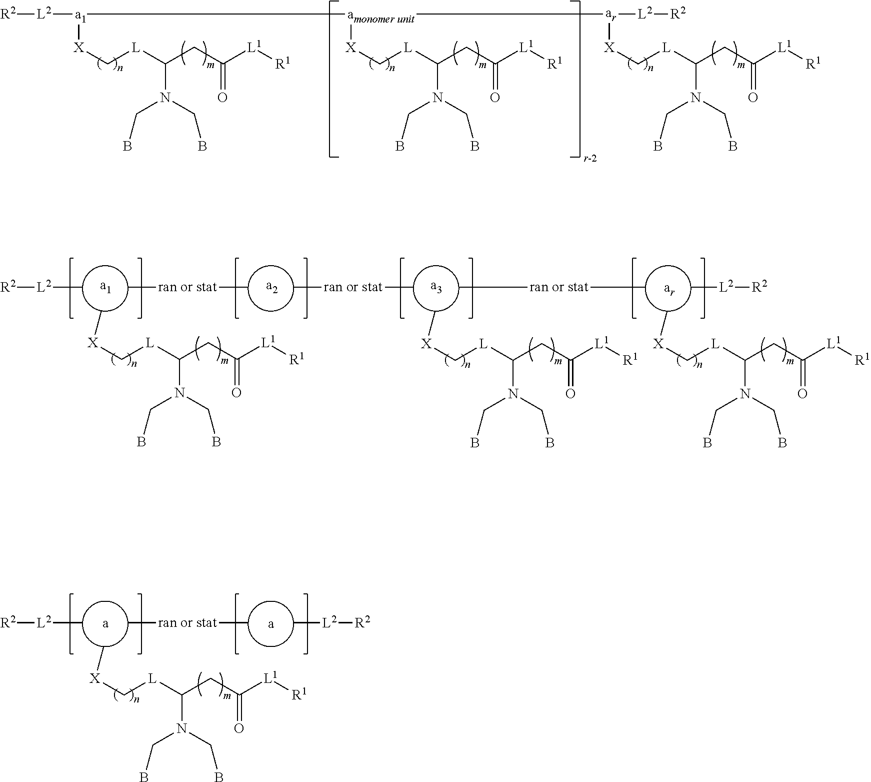

In some embodiments, the polymer backbone is a branched polymer, such as a hyperbranched polymer, or graft polymer. Exemplary representations of the compounds of Formula I include the structures shown below.

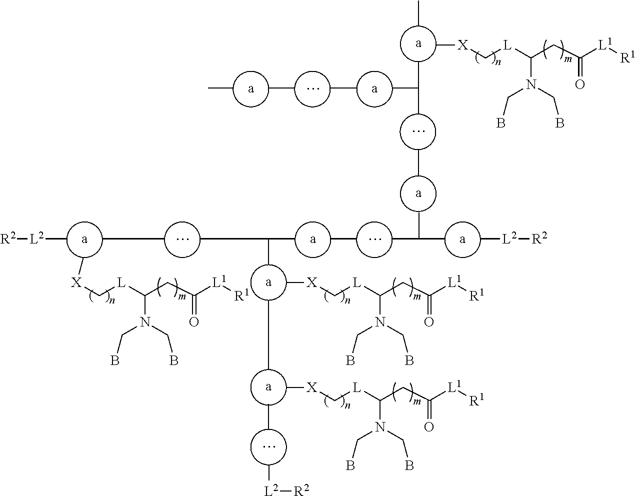

Each a (e.g. a₁, a₂, . . . , aᵣ) is a monomeric unit of the polymer backbone. The polymer backbone of the compound of Formula I can be a homopolymer or a copolymer. The copolymer can include graft copolymer or a block copolymer. It can be appreciated that each monomeric unit (e.g. a, a₁, a₂, . . . , aᵣ) can be the same for instance in a homopolymer, or different for example in a copolymer. For example, a₂ can be the same monomer as a₁, or a different monomer. Similarly, as can be the same monomer as a₂ and/or a₁, or a different monomer.

It is contemplated that at least one monomeric unit a is attached to a structure

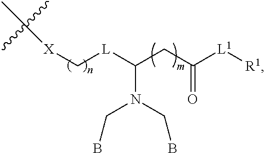

the latter capable of chelating to a metal, such as a soft metal. In some embodiments, each monomeric unit of the polymeric backbone is attached to

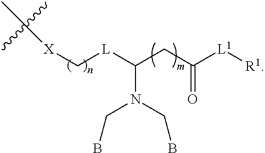

In some other embodiments, some of the monomeric units of the polymeric backbone, but not all, are attached to

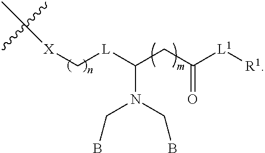

In some embodiments, the first modifying group R² can be present at the ends of the polymeric backbone. For example, each end of the polymeric backbone can be functionalised with a first modifying group R² through an optional linker L², each first modifying group R² and each linker L² being independently defined herein. In some embodiments, some of the ends of the polymeric backbone, but not all, can be functionalised with a first modifying group R² through an optional linker L², each first modifying group R² and each linker L² being independently defined herein.

In some embodiments, the polymer backbone can be a copolymer of monomers comprising different pendant groups. For example, it is envisioned that in a polyacrylamide backbone, the acrylamide monomers may be attached to a chelator pendant group or a modifying group such as a solubility modifying group or a reactive functional group. Exemplary polymer compounds of the present disclosure having a copolymer backbone are shown below.

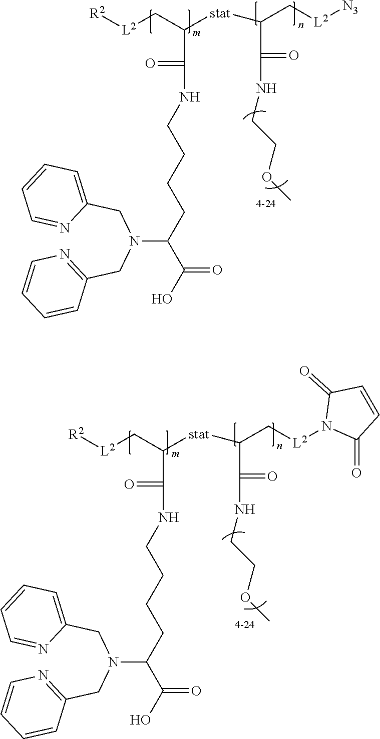

In some embodiments, the degree of polymerization (DP) can be approximately 1 to 1000 (1 to 2000 backbone atoms). Larger polymers are in the scope of the invention with the same functionality and are possible as would be understood by practitioners skilled in the art. Typically the degree of polymerization will be between 10 and 250. The polymers may be amenable to synthesis by a route that leads to a relatively narrow polydispersity. The polymer may be synthesized by atom transfer radical polymerization (ATRP), reversible addition-fragmentation (RAFT) polymerization or ring-opening polymerisation, which should lead to values of Mw/Mn in the range of 1.1 to 1.2. An alternative strategy involving anionic polymerization, where polymers with Mw/Mn of approximately 1.02 to 1.05 are obtainable. As such, the polymer may have a polydispersity index of 1.02 to 1.5, such as 1.02 to 1.2, 1.02 to 1.05, or 1.2 to 1.5. These methods permit control over end groups, through a choice of initiating or terminating agents. This allows synthesizing polymers to which the linker can be attached. A strategy of preparing polymers containing functional pendant groups in the repeat unit to which the liganded transition metal unit (for example a soft metal unit) can be attached in a later step can be adopted. This embodiment has several advantages. It avoids complications that might arise from carrying out polymerizations of ligand-containing monomers. In addition, the polymer backbone is a known one that can be adapted for most if not all of the soft-metal-containing polymers. Thus, the polymers may have a common mean chain length and chain-length distribution.

In some embodiments, each linker independently comprises or is independently selected from C3-C8 alkyl amine, C3-C8 alkylene, C3-C8 cycloalkyl, C3-C8 heterocycloalkyl, 5-membered or 6-membered aryl or heteroaryl, alkylaryl, alkylheteroaryl, C3-C8 cycloalkylaryl, C3-C8 cycloalkylheteroaryl, C(O), C(O)O, amide, amine, thioether, maleimide-thiol conjugate, polyethylene glycol (PEG), or mixtures thereof, optionally each of the amine, alkylene, aryl, alkylaryl, alkylheteroaryl, cycloalkyl, cycloalkylaryl, and cycloalkylheteroaryl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

In some embodiments, each L² independently comprises or is independently selected from C3-C8 alkylene, C3-C8 alkyl amine, ester, amine, amide, thioether, maleimide-thiol conjugate, PEG, or mixtures thereof, optionally each of the alkylene and alkyl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

In some embodiments, each L¹ independently comprises or is independently selected from C3-C8 alkylene, C3-C8 alkyl amine, ester, amine, amide, thioether, maleimide-thiol conjugate, PEG, or mixtures thereof, optionally each of the alkylene and alkyl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

In some embodiments, L is absent or a C3-C8 alkyl amine.

In some embodiments, it is understood that a linker can include the functional group that attaches the linker to the remaining of the compound.

A biomolecule may be classified as a protein, an oligonucleotide, a lipid, a carbohydrate, or a small molecule or combinations thereof. Alternatively or in addition, a biomolecule may be classified by its functionality. The biomolecule is not particularly limited and different functionalizations can be used to conjugate the biomolecule to the compounds of the present disclosure. For example, an oligonucleotide may be a single stranded DNA molecule, optionally cDNA that hybridizes under stringent conditions to a target nucleic acid analyte (e.g. a sample nucleic acid biomolecule) or the oligonucleotide can be an aptamer. For example, a biomolecule may be an oligonucleotide that specifically hybridizes a target oligonucleotide, such as a target mRNA endogenous to a sample (e.g. hybridizes to the sample oligonucleotide). Hybridization may be of a sequence that is more than 8, more than 10, more than 15, or more than 20 nucleotides.

In certain aspects, a biomolecule may be classified by its functionality. For example, a biomolecule may be an affinity reagent, an antigen (e.g., an analyte specifically bound by an affinity reagent), or an enzyme substrate. An affinity reagent may be an antibody (e.g., or fragment thereof), aptamer, receptor (e.g., or portion thereof), or any other biomolecule that specifically binds a target (e.g., an avidin, such as streptavidin, that specifically binds biotin). For example, an element tag may be associated with an antibody may be used to detect and/or analyze the presence of its target antigen in a sample, such as the presence of a cytokine, viral protein, cancer biomarker, or the like. In certain methods and kits, an element tag may be functionalized with an avidin for attachment of another biomolecule functionalized with biotin (e.g., to allow a compound of the present disclosure to be adapted to any of a number of different assays). An antigen may be a protein (or peptide sequence thereof) comprising an epitope that is specifically bound by an affinity reagent such as an antibody. For example, a compound of the present disclosure may be attached to a viral antigen (such as a viral protein sequence), and may be used to detect the presence of antibodies in the sample that specifically bind the viral antigen, as described further herein. An enzyme substrate may be any substrate that is acted on by a specific enzyme, such as by an oxidoreductase, transferase, hydrolase, lyase, isomerase or ligase. For example, a substrate may be a protein (e.g., or a peptide sequence thereof) that is a substrate for an enzyme such as a protease, phosphatase, kinase, methyltransferase, demethylases. Non-protein substrates include, for example, a double stranded oligonucleotide comprising a restriction sequence cleavable by a restriction enzyme or a site (such as a nick) for DNA repair, an oligonucleotide sequence comprising a sequence targeted by a DNA methyltransferase, or any non-protein substrate known to one of skill in the art. For example, a compound of the present disclosure may be attached to a substrate and exposed to a sample comprising an enzyme that modifies the substrate, and modification (or lack thereof) of the substrate may be detected (e.g., as described further herein).

In some embodiments, the one or more biomolecules are each independently selected from a small molecule, a polypeptide, an oligonucleotide, a lipid, a carbohydrate, or a mixture thereof.

In some embodiments, the one or more biomolecules are each independently an affinity reagent, optionally, wherein the affinity reagent is an antibody.

In some embodiments, the affinity reagent is or comprises an antibody or a binding fragment thereof. The antibody can for example be a biotinylated antibody or binding fragment and can be added directly or indirectly to the compound of present disclosure.

In some embodiments, the compound of Formula I is selected from

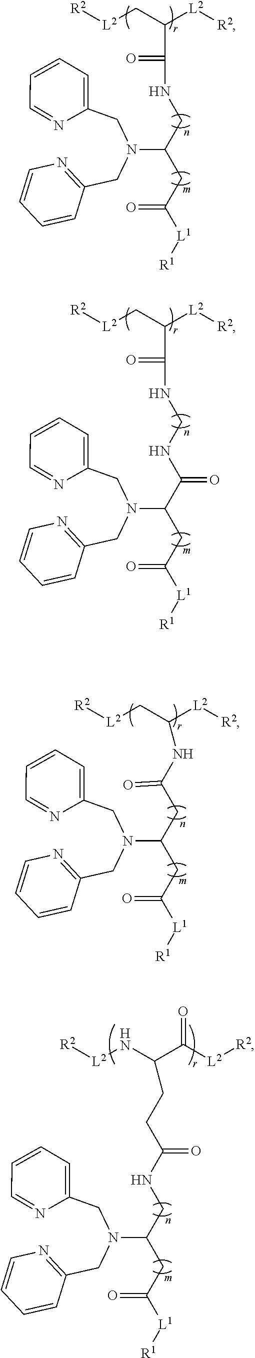

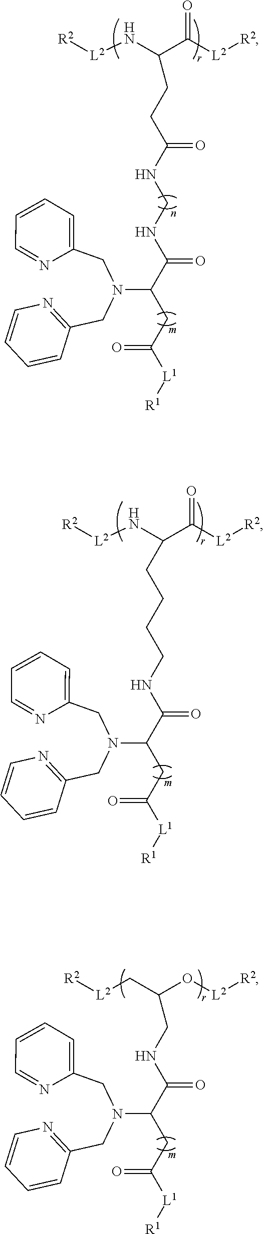

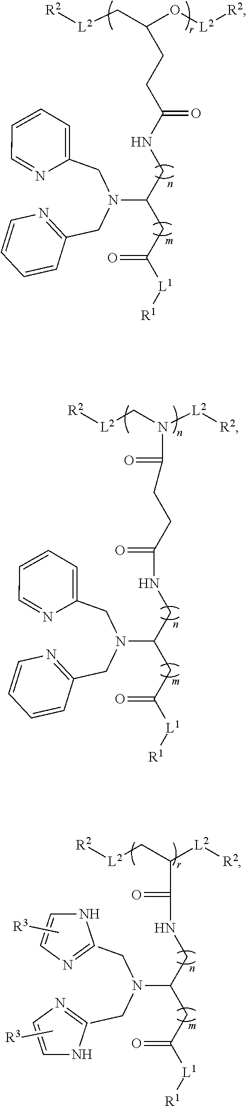

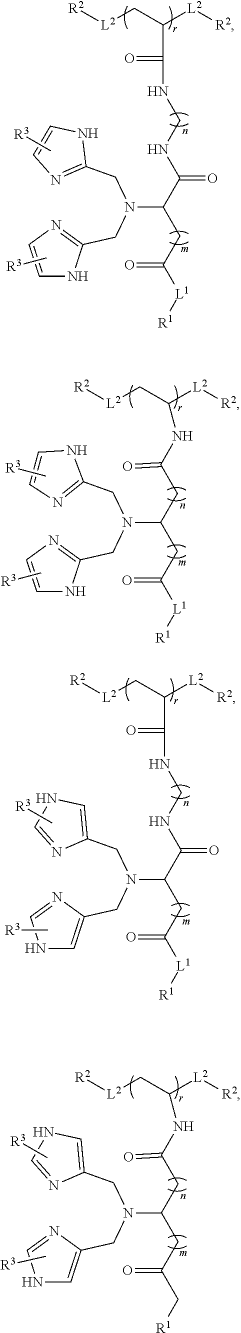

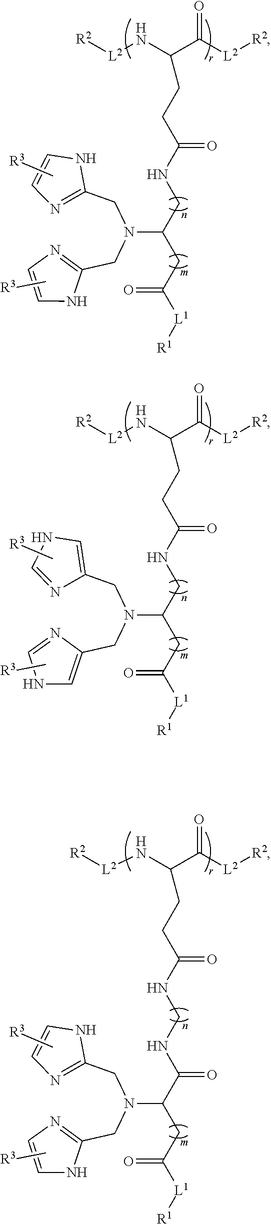

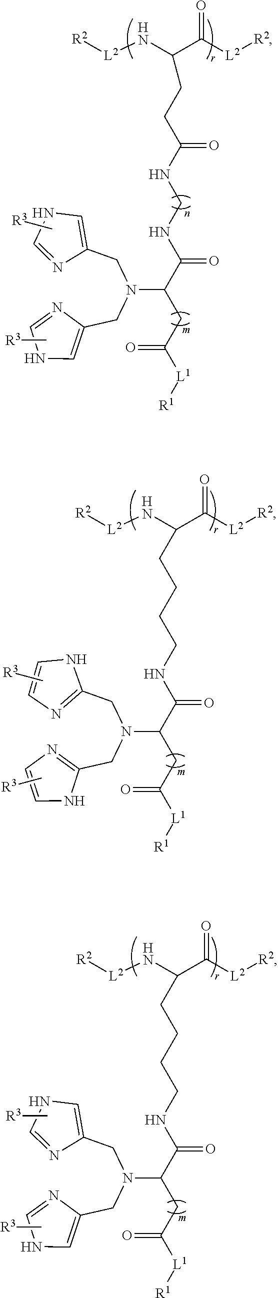

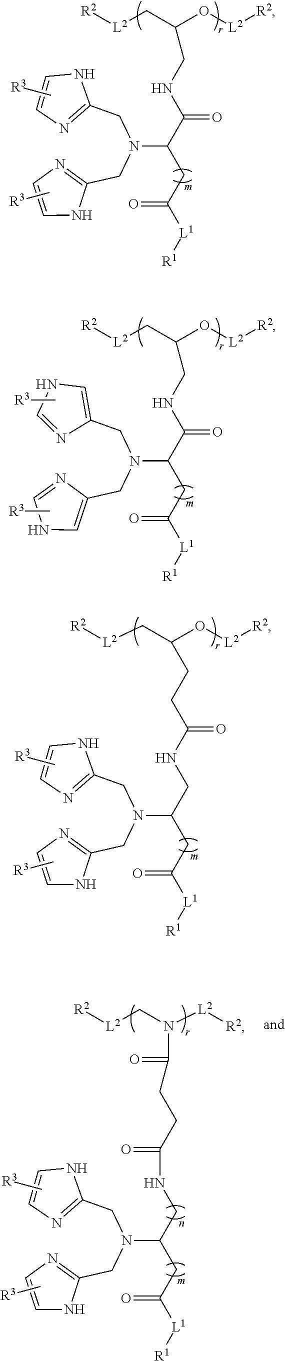

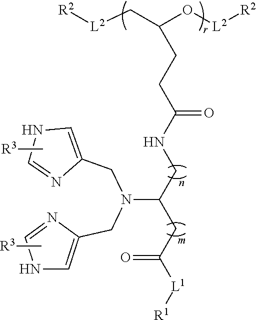

wherein r is about 3 to about 300, about 3 to about 250, about 3 to about 200, about 3 to about 150, about 3 to about 100, about 3 to about 50, about 6 to about 30, or about 10 to about 25, and wherein R¹, R², L¹, L² and R³ are each as defined herein. In some embodiments, r is up to 300, up to 250, up to 200, up to 150, up to 100, up to 50, up to 30, or up to 25. In some embodiments, r is at least 3, at least 6, or at least 10.

In some embodiments, the compound of Formula I is selected from

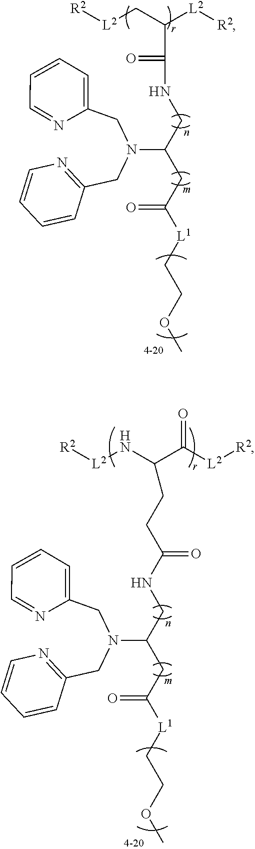

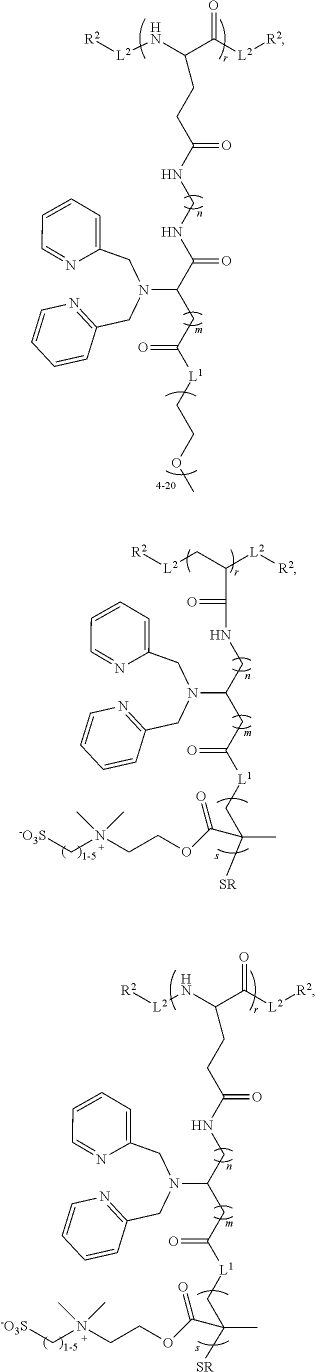

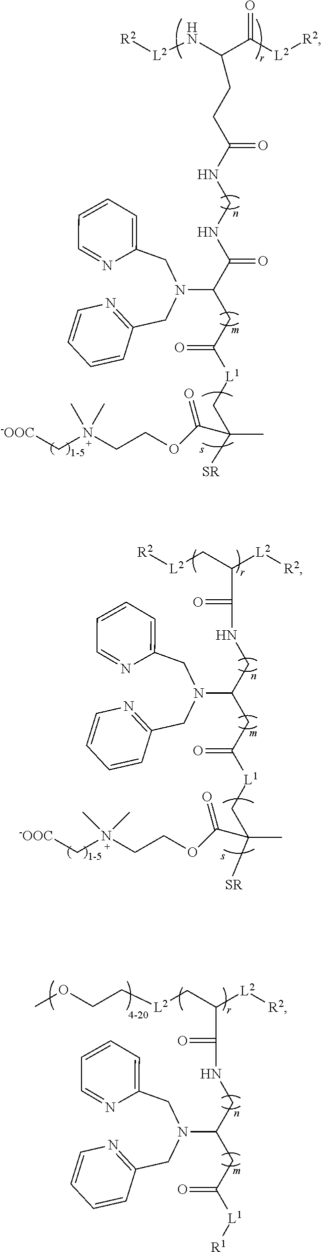

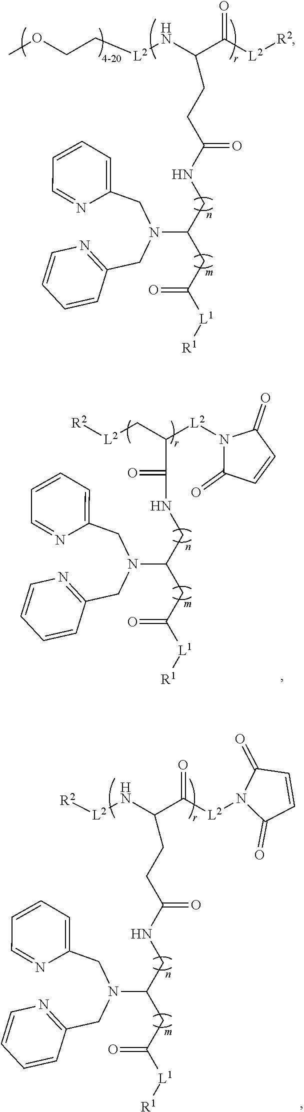

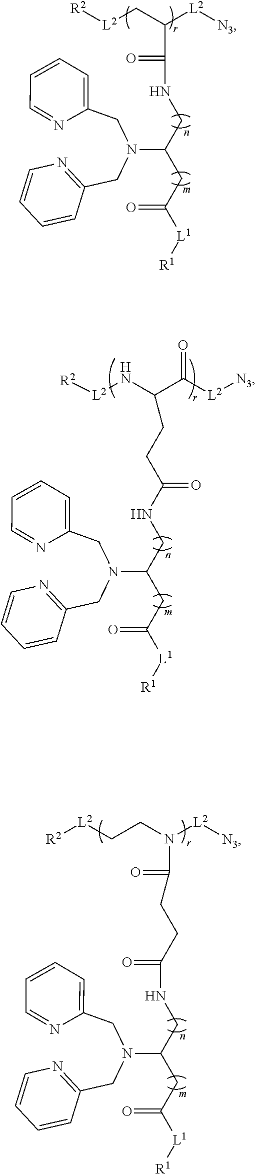

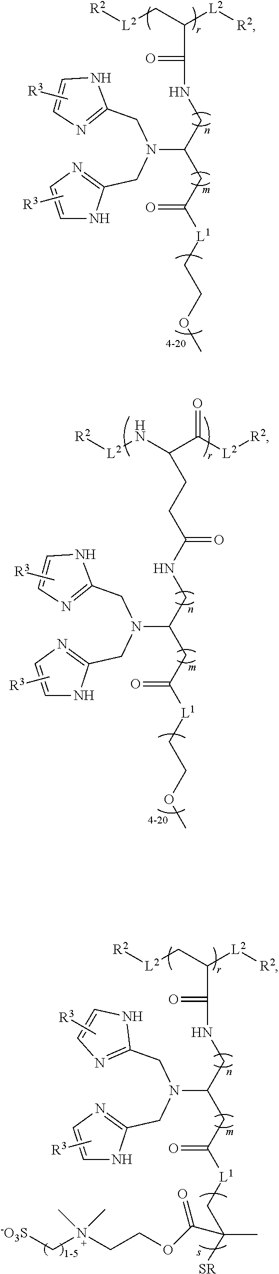

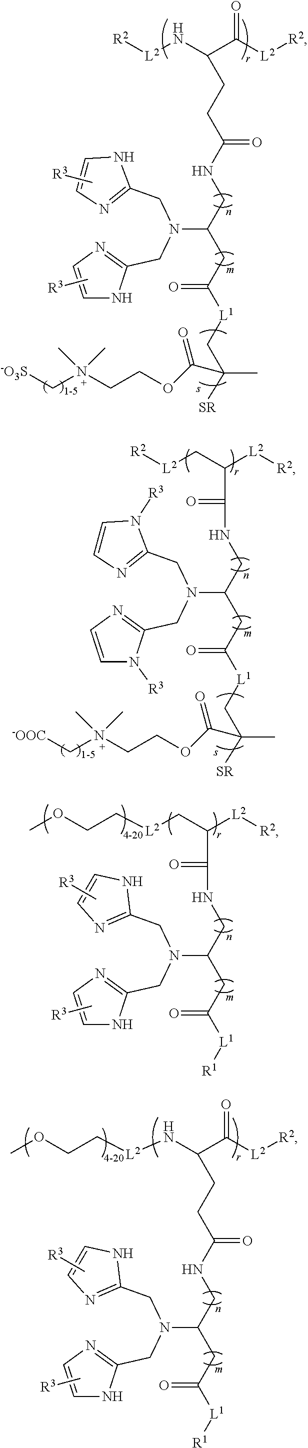

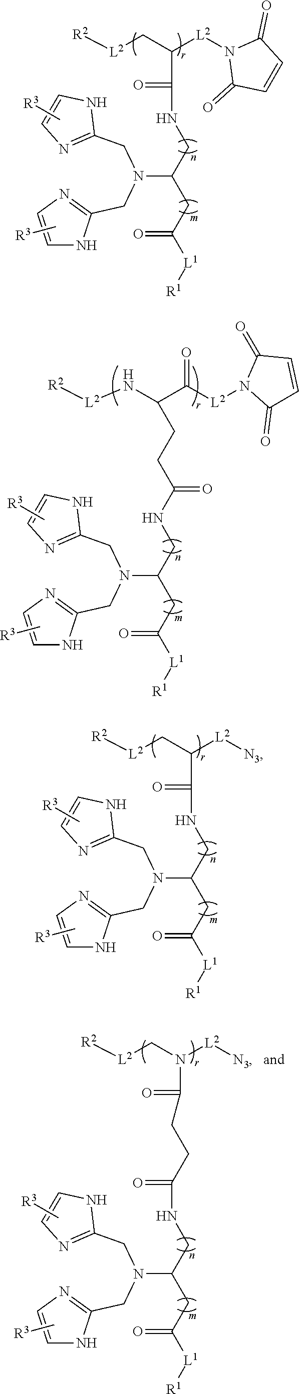

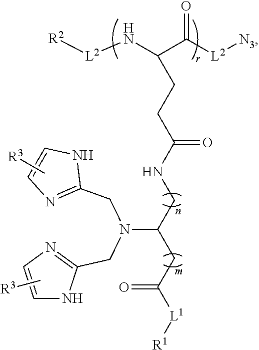

wherein s is about 1 to about 50, about 2 to about 40, about 5 to about 30, about 10 to about 30, about 5 to about 35, or about 20 to about 30, and r is about 3 to about 300, about 3 to about 250, about 3 to about 200, about 3 to about 150, about 3 to about 100, about 3 to about 50, about 6 to about 30, or about 10 to about 25, and wherein R¹, R², L¹, L² and R³ are each as defined herein. In some embodiments, r is up to 300, up to 250, up to 200, up to 150, up to 100, up to 50, up to 30, or up to 25. In some embodiments, r is at least 3, at least 6, or at least 10.

In some embodiments, the compound of Formula I is selected from

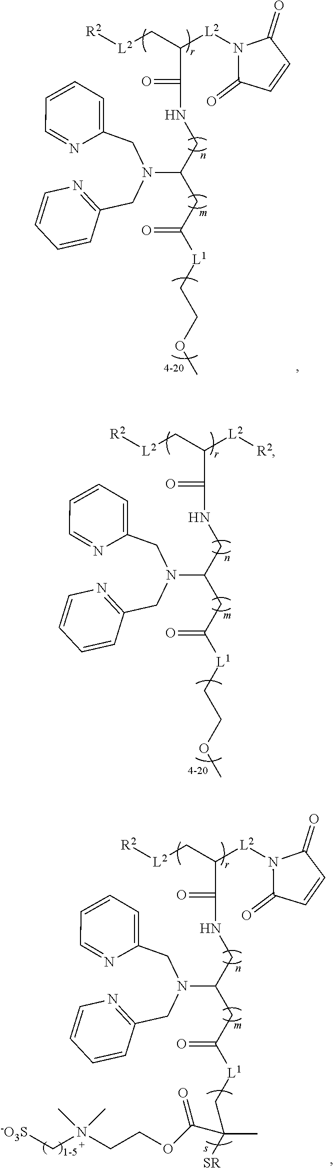

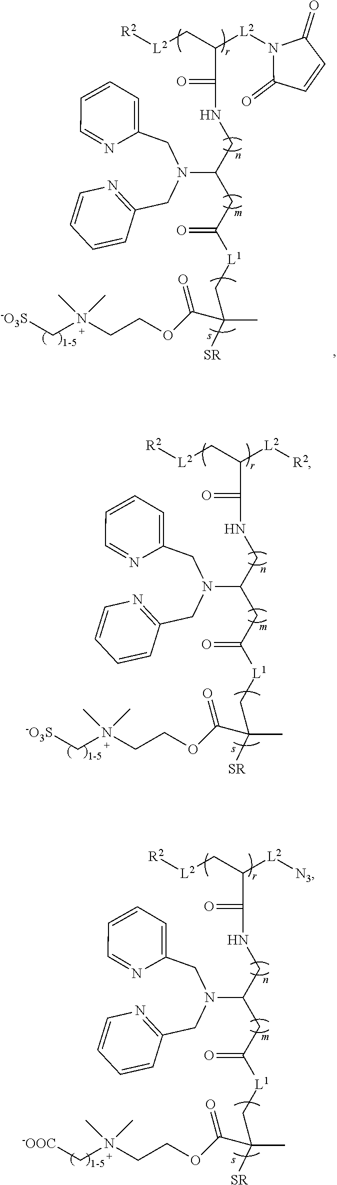

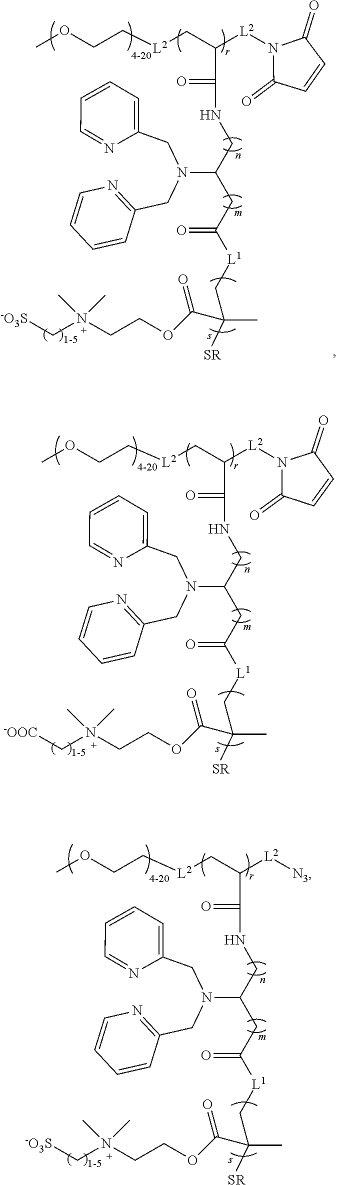

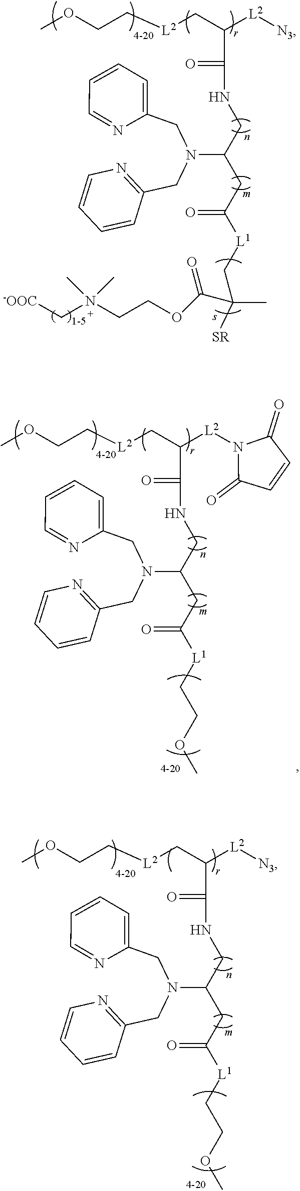

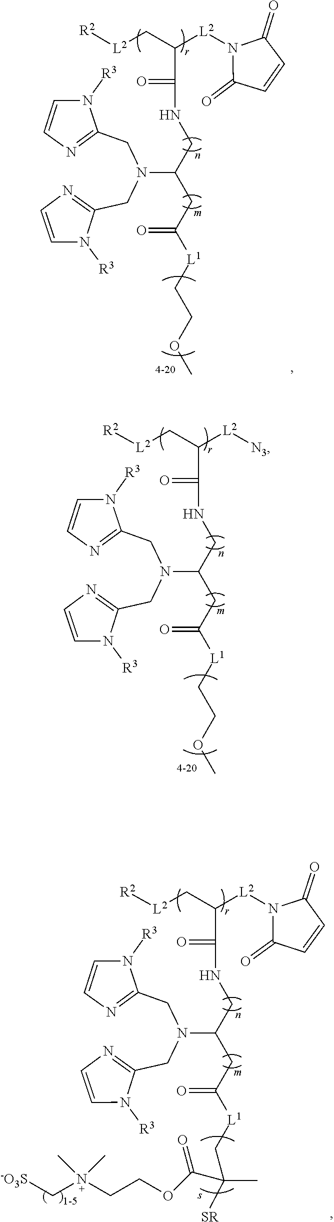

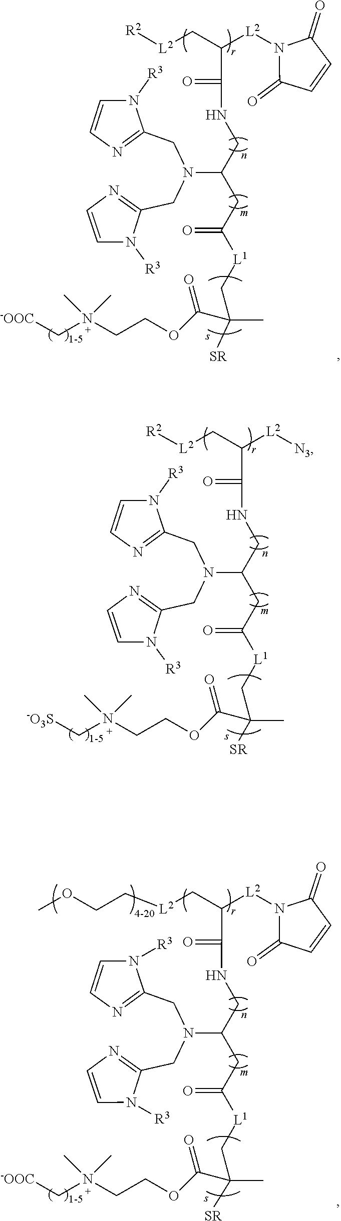

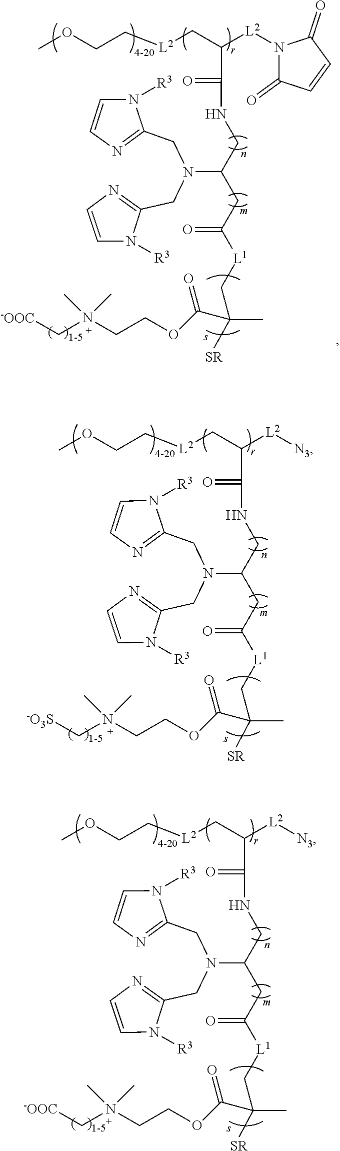

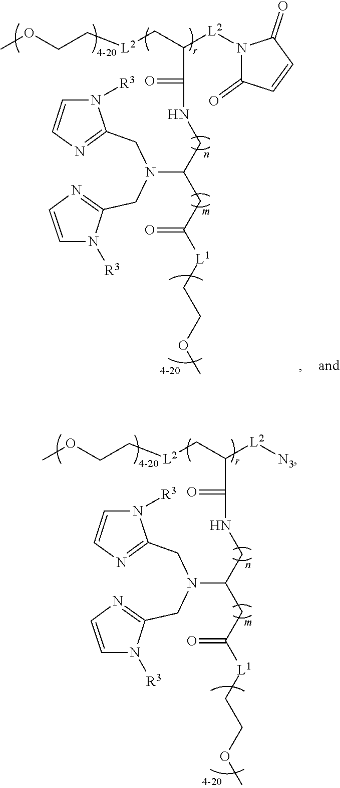

wherein s is about 1 to about 50, about 2 to about 40, about 5 to about 30, about 10 to about 30, about 5 to about 35, or about 20 to about 30, and r is about 3 to about 300, about 3 to about 250, about 3 to about 200, about 3 to about 150, about 3 to about 100, about 3 to about 50, about 6 to about 30, or about 10 to about 25, and wherein R¹, R², L¹, L² and R³ are each as defined herein. In some embodiments, r is up to 300, up to 250, up to 200, up to 150, up to 100, up to 50, up to 30, or up to 25. In some embodiments, r is at least 3, at least 6, or at least 10.

In some embodiments, the compound of Formula I is selected from

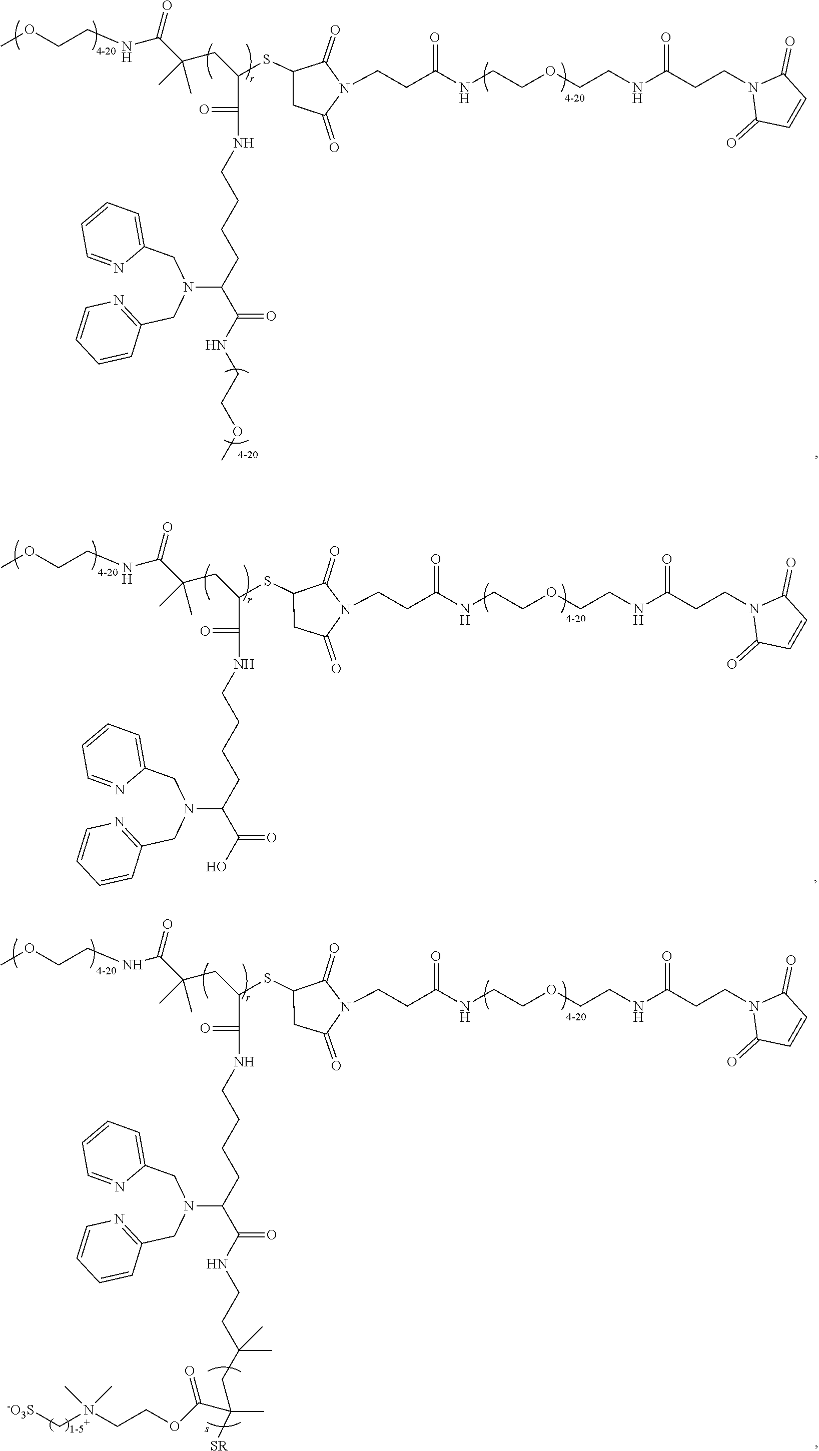

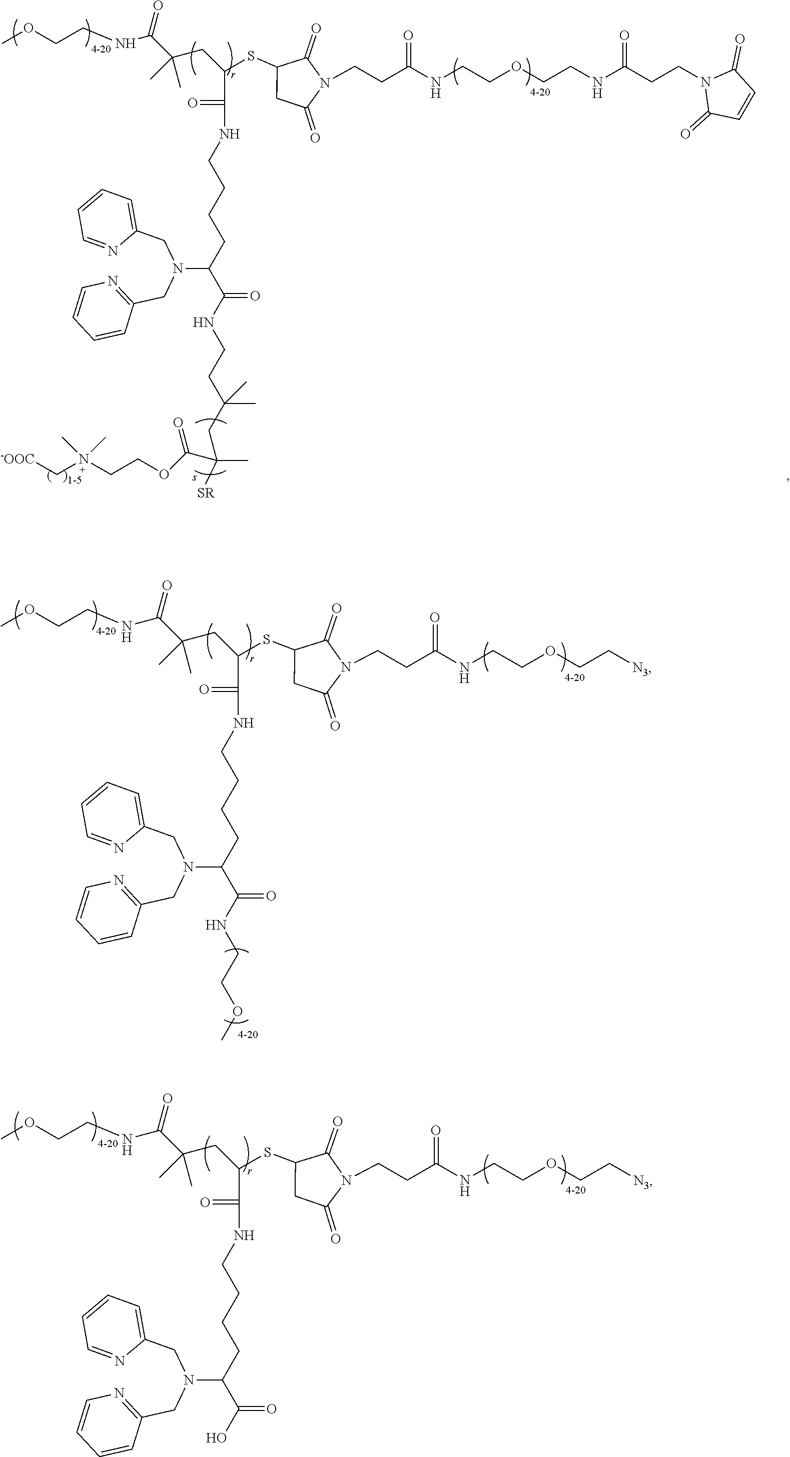

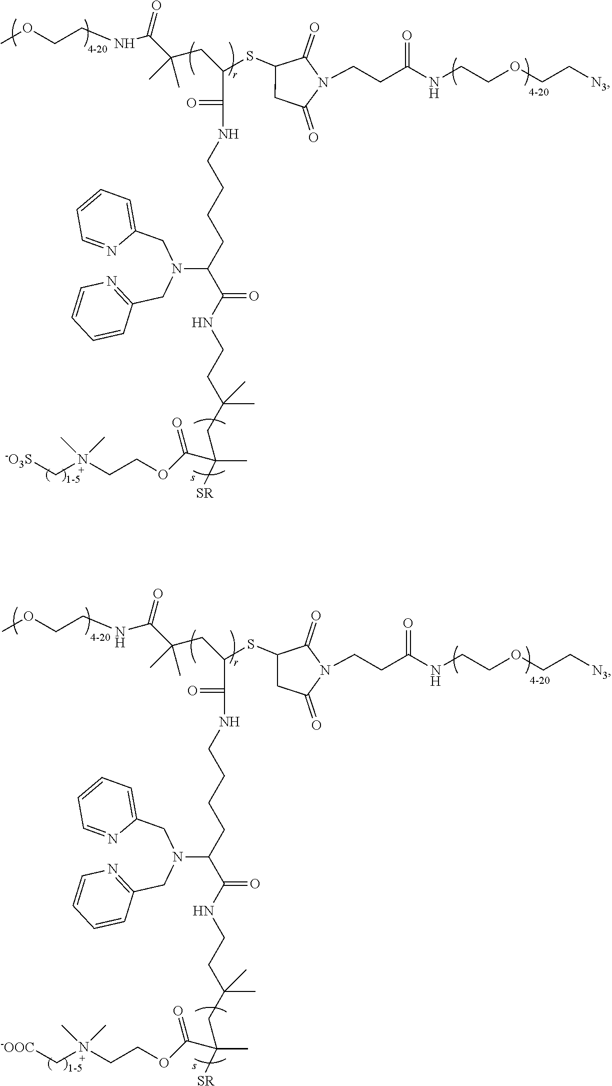

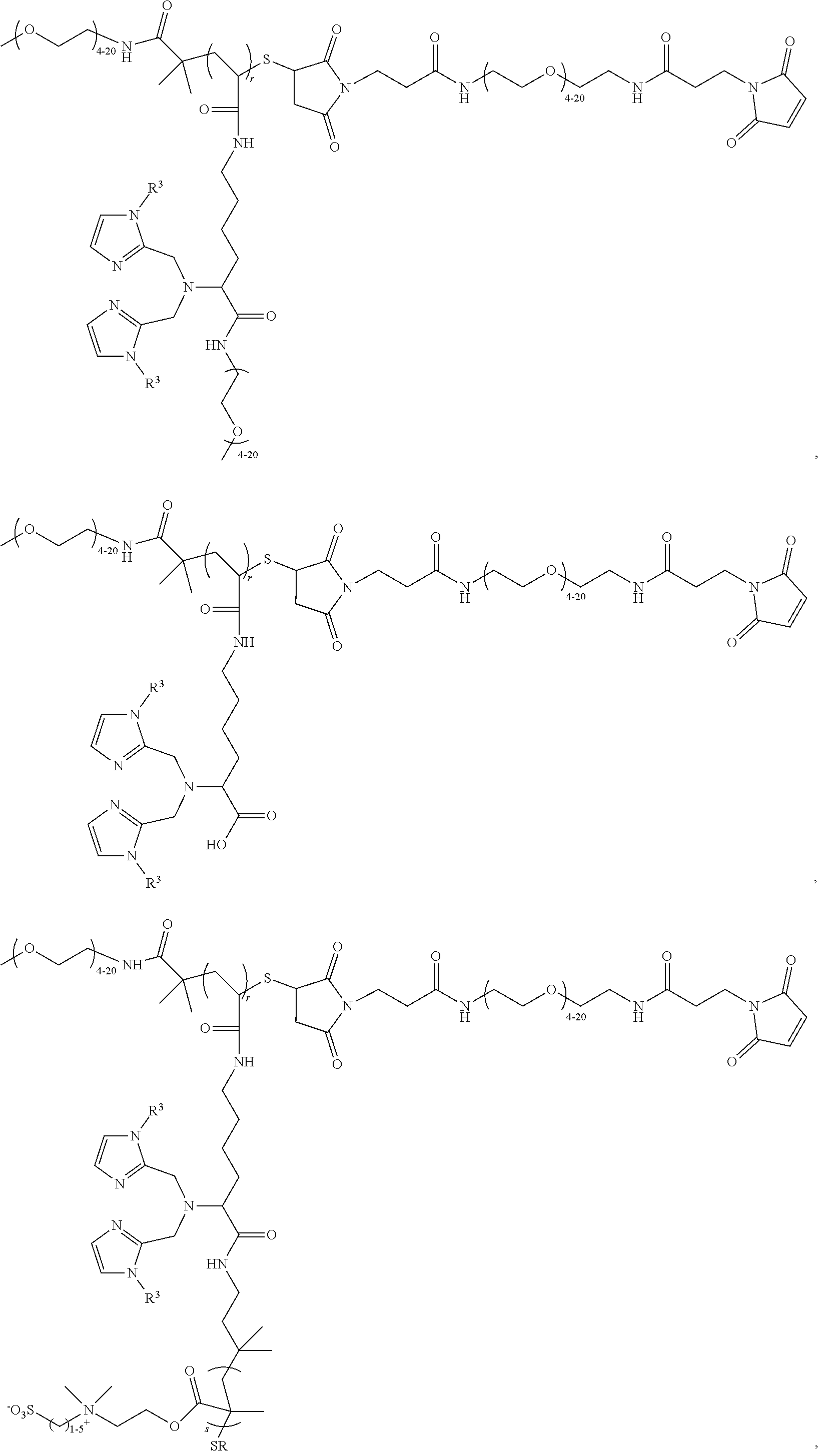

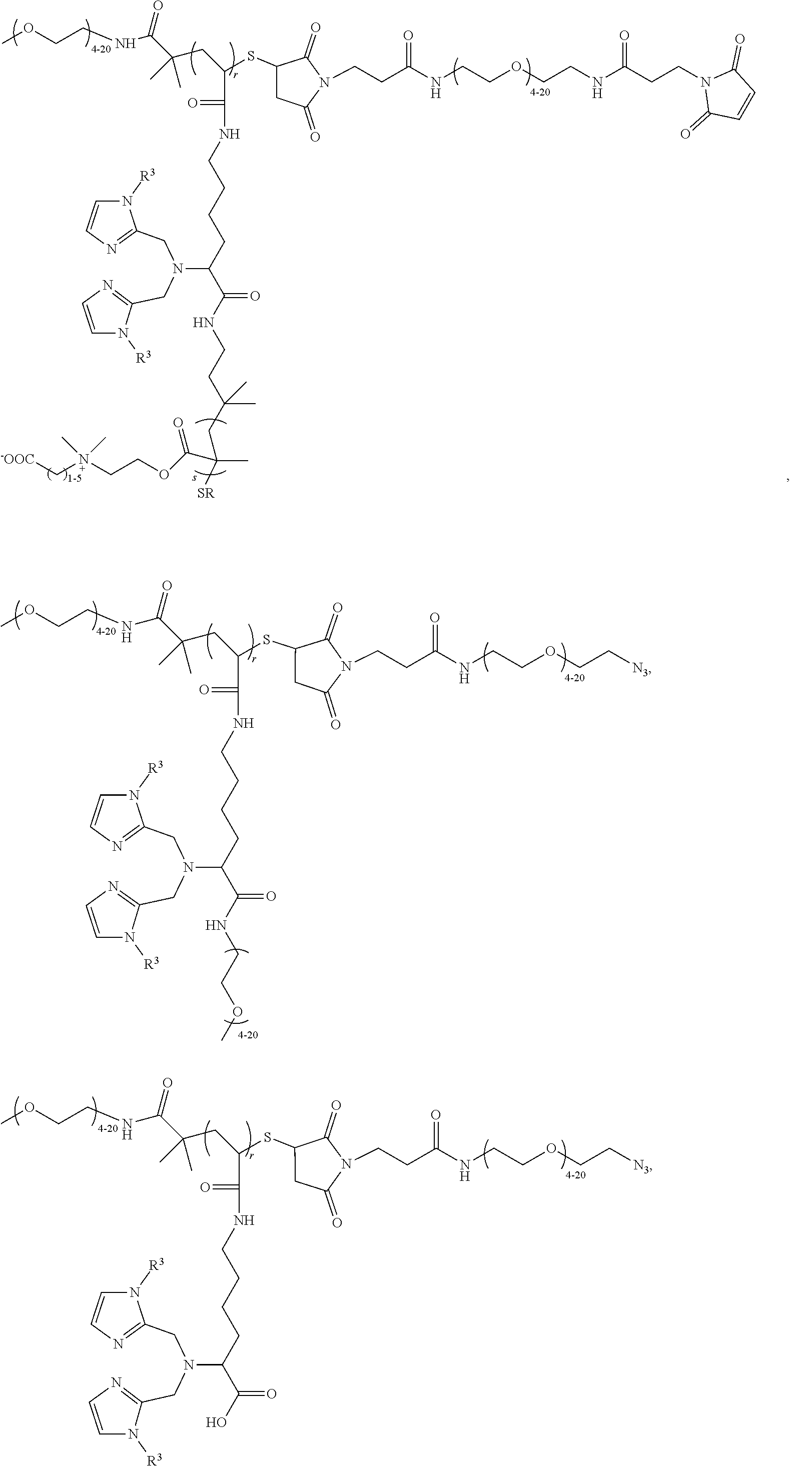

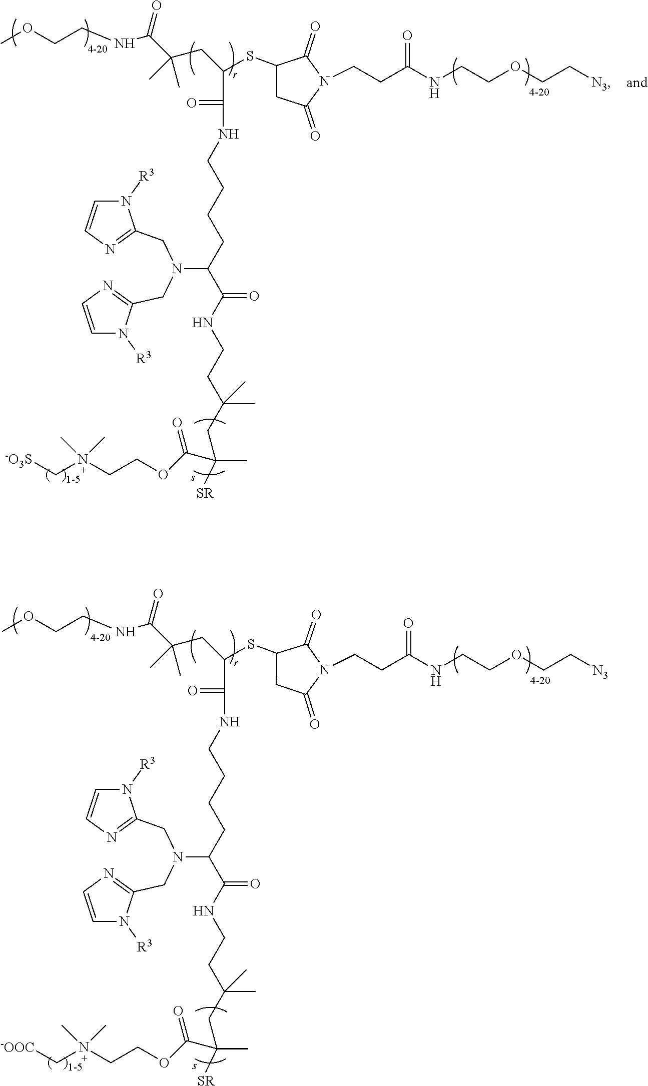

wherein s is about 1 to about 50, about 2 to about 40, about 5 to about 30, about 10 to about 30, about 5 to about 35, or about 20 to about 30, and r is about 3 to about 300, about 3 to about 250, about 3 to about 200, about 3 to about 150, about 3 to about 100, about 3 to about 50, about 6 to about 30, or about 10 to about 25, and wherein R³ is as defined herein. In some embodiments, r is up to 300, up to 250, up to 200, up to 150, up to 100, up to 50, up to 30, or up to 25. In some embodiments, r is at least 3, at least 6, or at least 10.

In some embodiments, the compound of Formula I is selected from

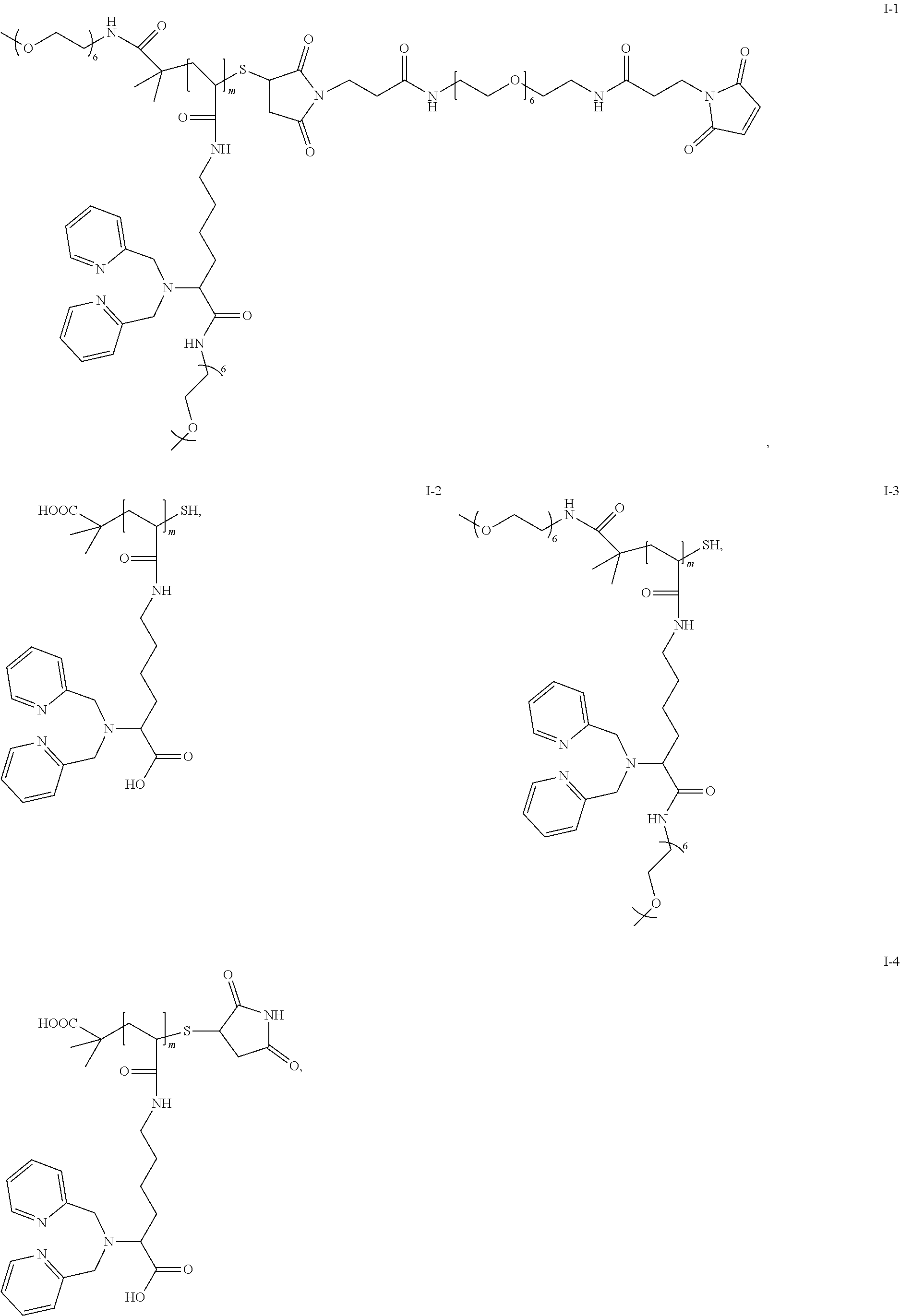

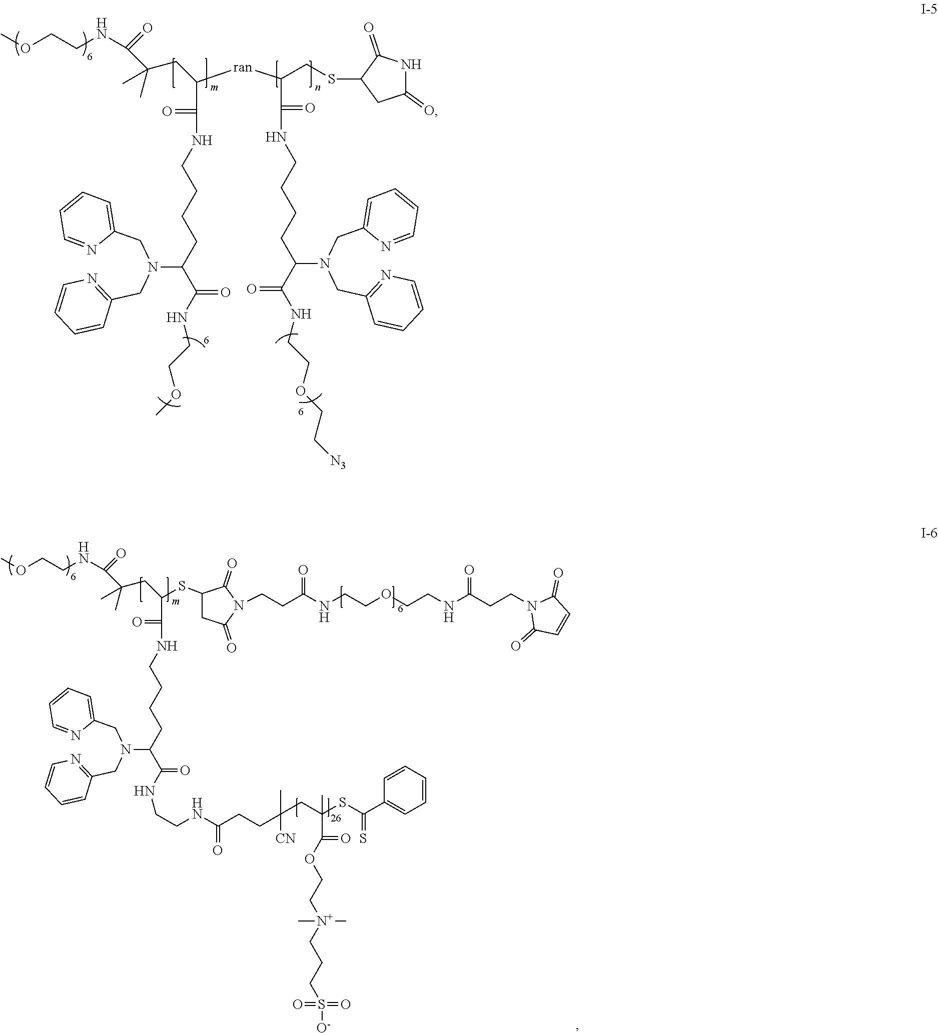

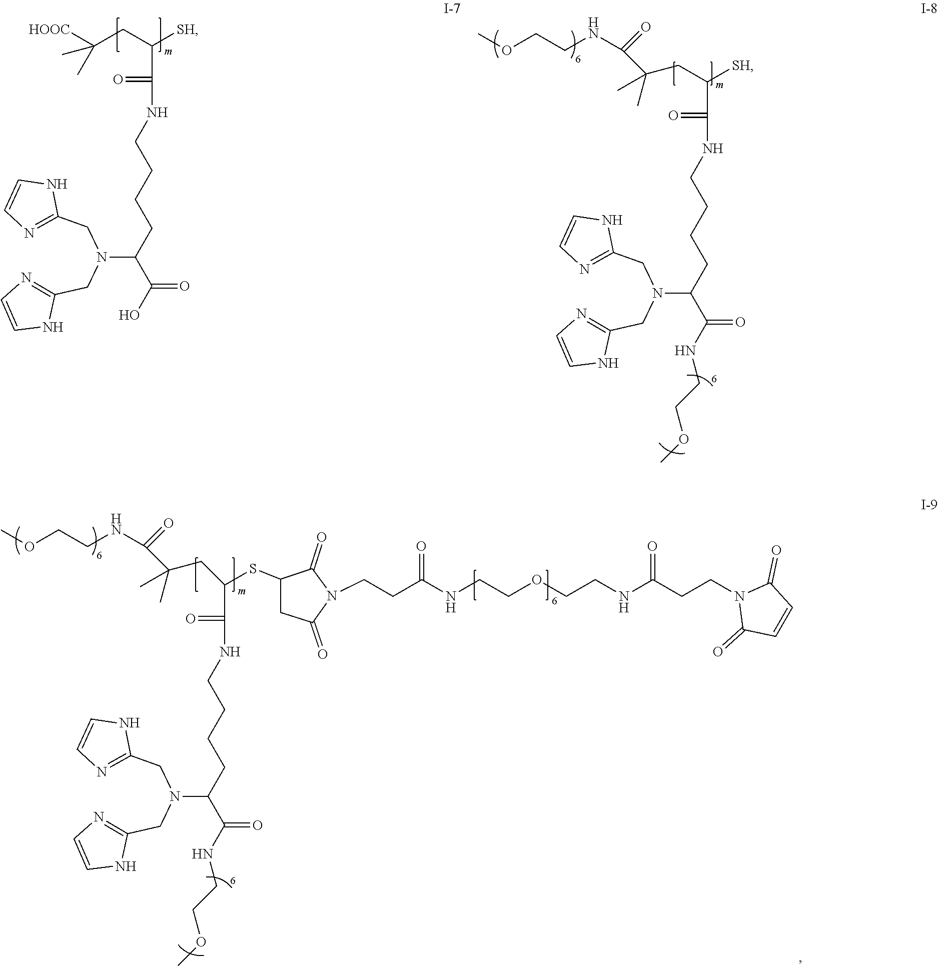

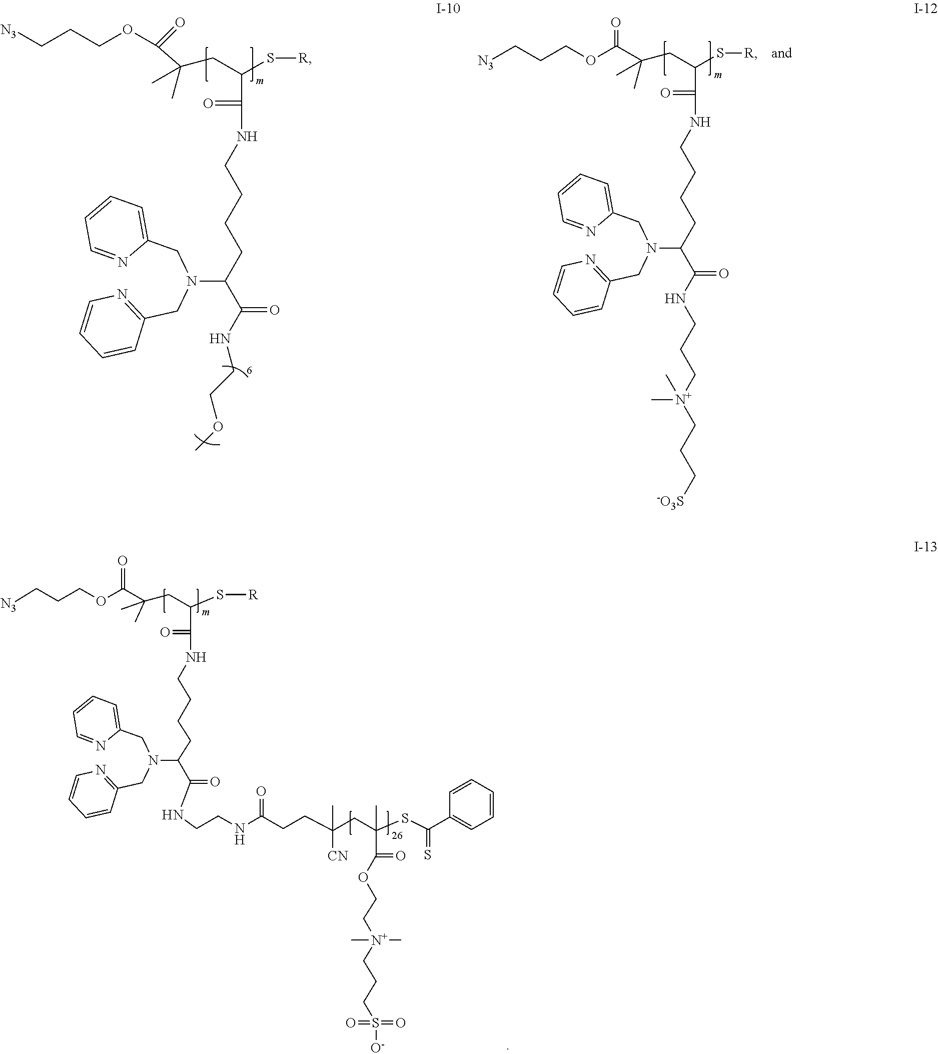

In some embodiments, the compound of Formula II is selected from

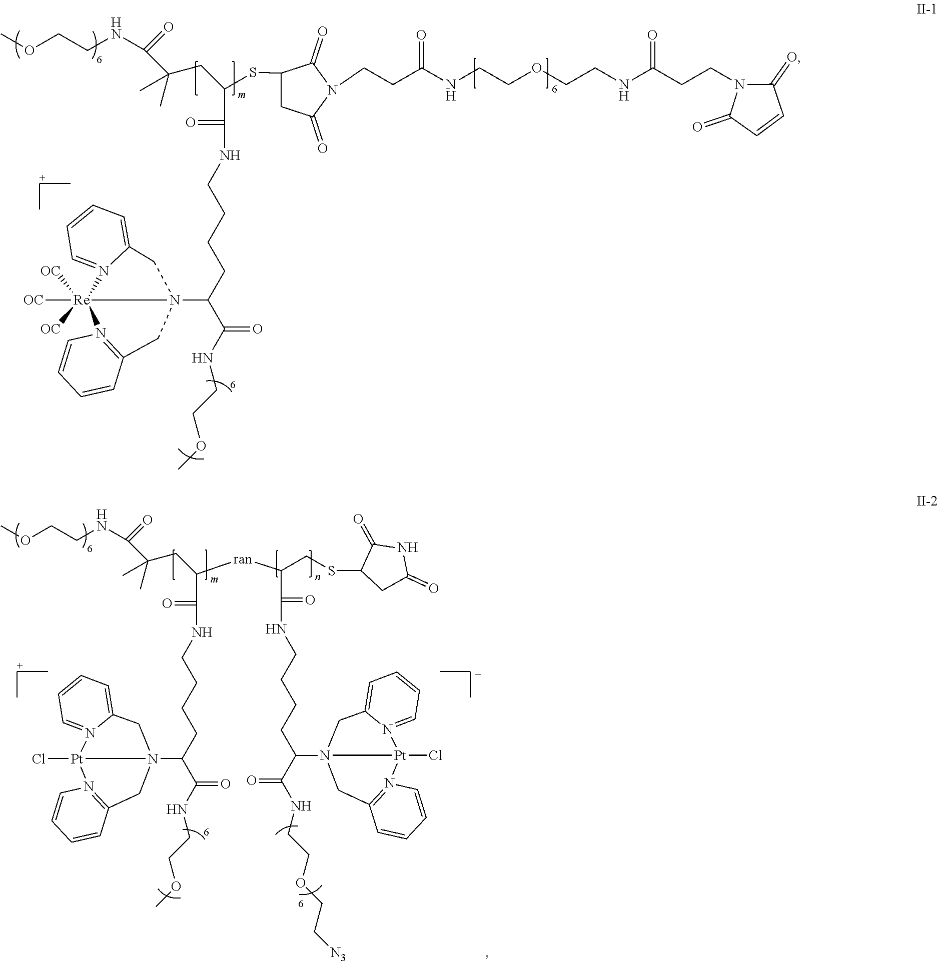

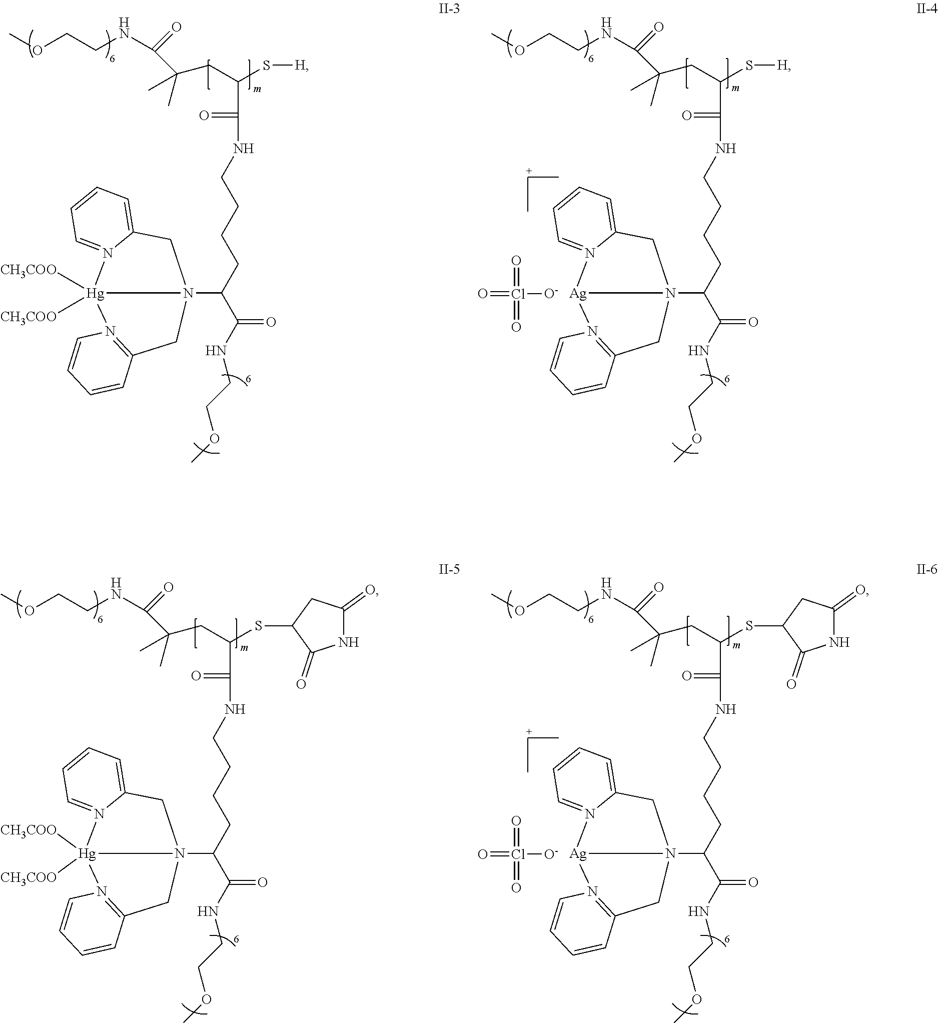

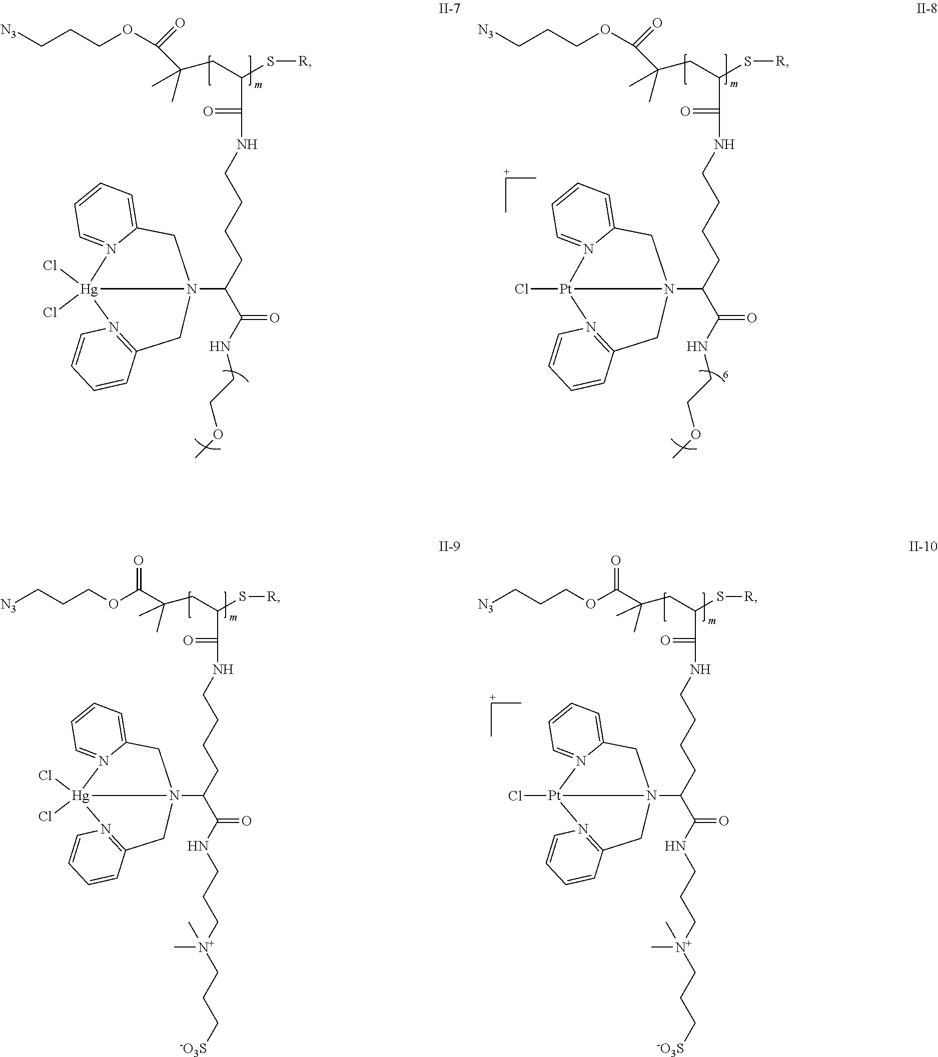

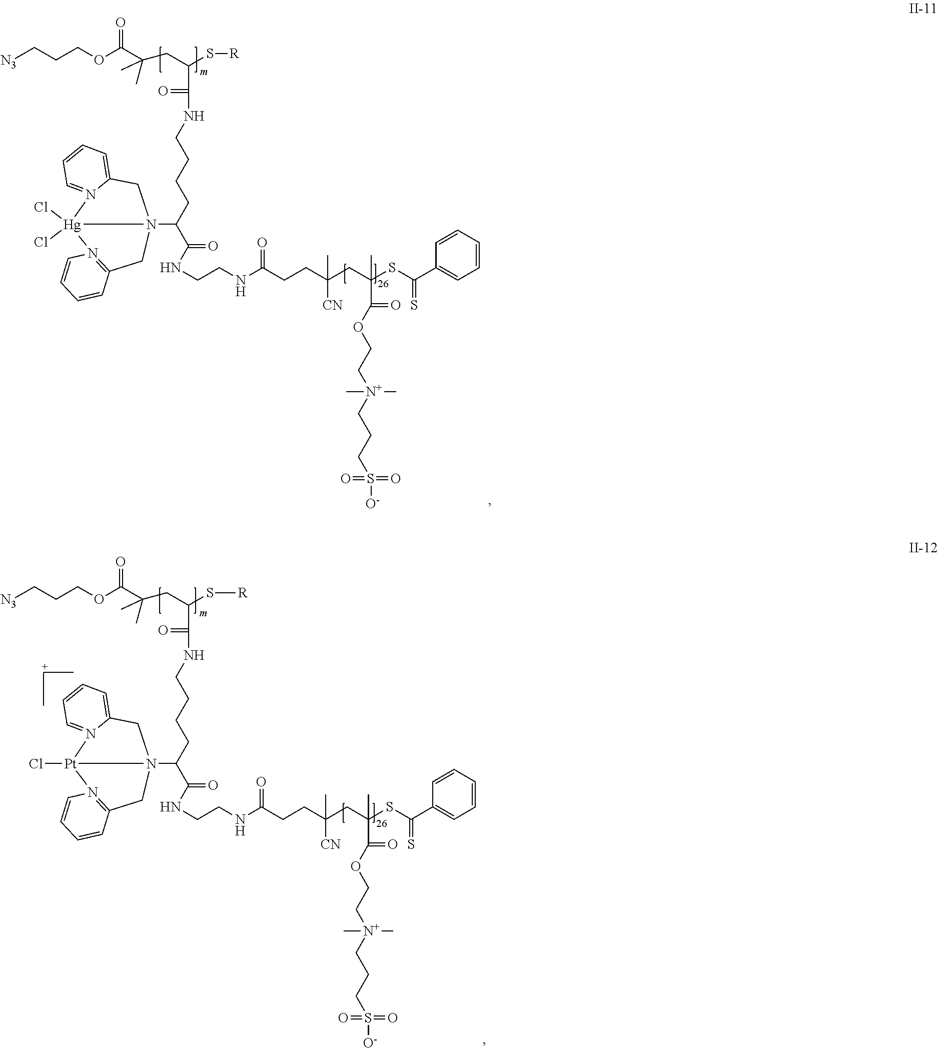

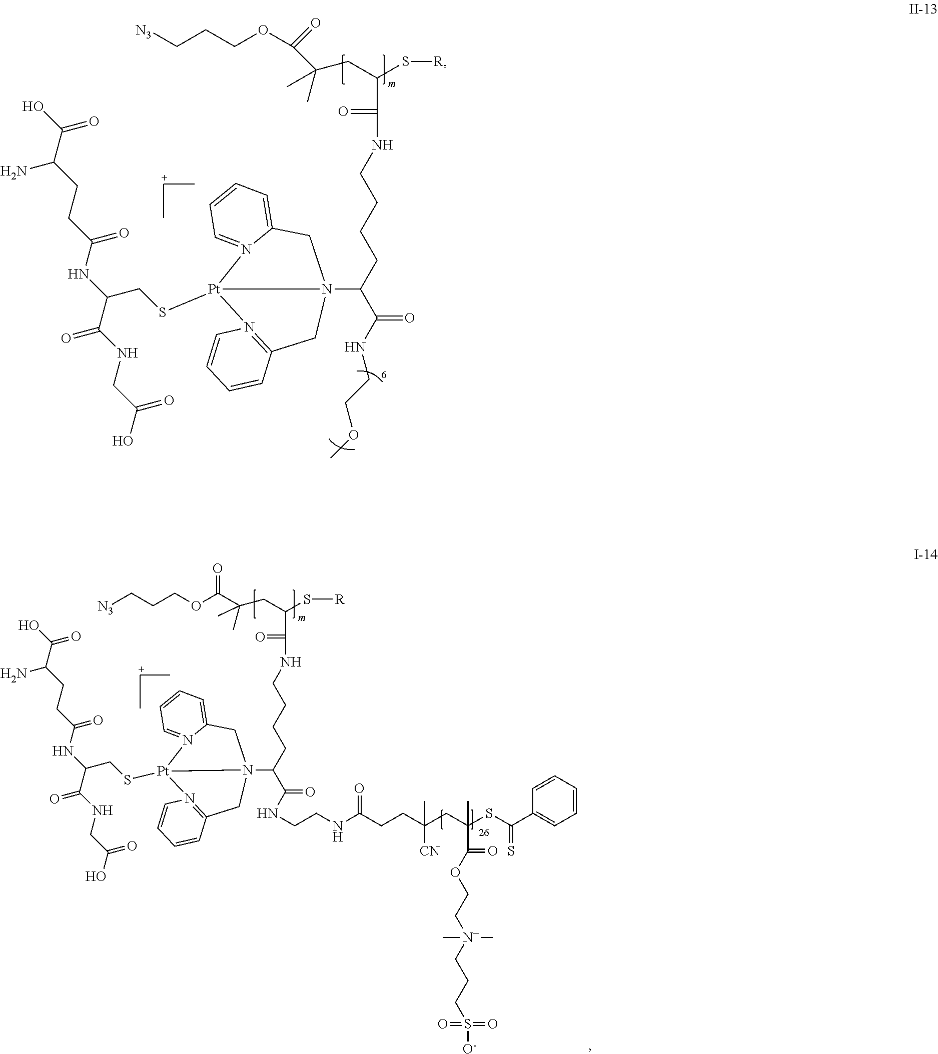

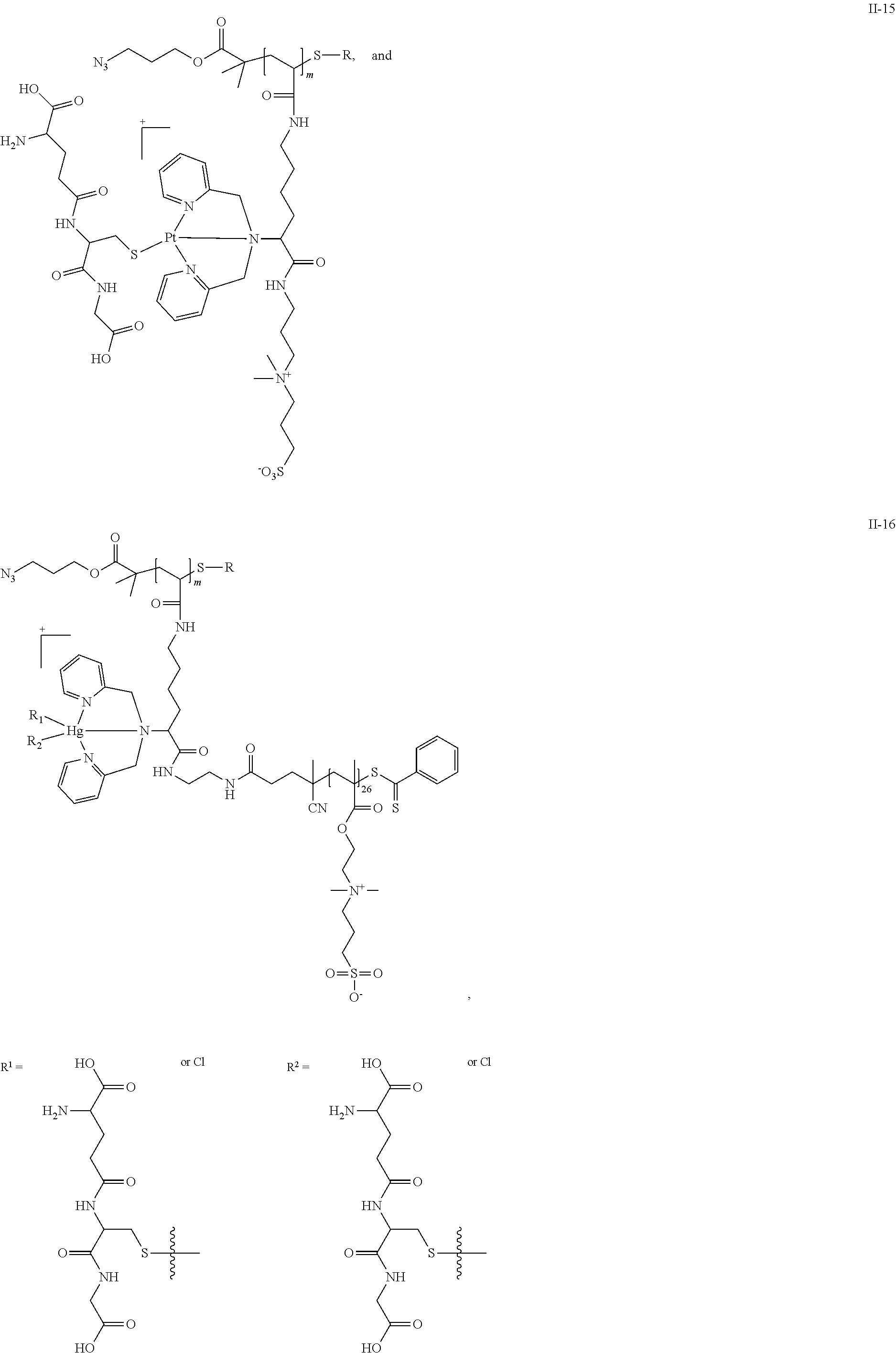

wherein R is H,

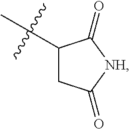 [SMILES: *C1CC(=O)NC1=O]

or other suitable thiol capping group known in the art. In some embodiments, R signifies a second compound of Formula II forming a dimer through disulfide bond.

It can be appreciated that bis-heterocyclic chelators such as DPA, bis((1-methyl-imidazol-4-yl-)methyl)amine, bis((1-methyl-imidazol-2-yl-)methyl)amine, bis((1H-imidazol-4-yl-)methyl)amine and bis((1H-imidazol-2-yl-)methyl)amine can stably chelate metals, especially soft metals. Polymer compounds of the present disclosure comprising one or more pendant groups including bis-heterocyclic chelators such as DPA, bis((1-methyl-imidazol-4-yl-)methyl)amine, bis((1-methyl-imidazol-2-yl-)methyl)amine, bis((1H-imidazol-4-yl-)methyl)amine and bis((1H-imidazol-2-yl-)methyl)amine can chelate metals including soft metals. When chelated to a metal, the compounds of the present disclosure introduces new mass channels to applications such as mass cytometry represented by the stable isotopes of the metal.

The Examples herein show exemplary chelates formed using the polymer compounds of the present disclosure with metals including Re, Pt, Hg, and Ag. It is known that chelators such as DPA and can form stable chelates with other soft metals in non-polymer context. Accordingly, polymer compounds of the present disclosure bearing chelators such as DPA, bis((1-methyl-imidazol-4-yl-)methyl)amine, bis((1-methyl-imidazol-2-yl-)methyl)amine, bis((1H-imidazol-4-yl-)methyl)amine, and bis((1H-imidazol-2-yl-)methyl)amine can chelate to other soft metals as well.

For example, Seubert et al. [“Chimeric GNA/DNA metal-mediated base pairs”, Chem. Commun., 2011, 47, 11041-11043] reported computational analysis of Au(III) chelated to DPA. Messori et al. [J. Med. Chem. 2000, 43, 3541-3548] reported a series of Au(III) complexes with diamines and triamines.

Molybdenum complex with DPA [Mo(dipic)(CO)₃] has been reported by van Staveren et al, Labelling of [Leu5]-enkephalin with organometallic Mo complexes by solid-phase synthesis, Chem. Commun., 2002, 1406-1407.

Minard et al. [In situ generation of water-stable and -soluble ruthenium complexes of pyridine-based chelate-ligands and their use for the hydrodeoxygenation of biomass-related substrates in aqueous acidic medium, Journal of Molecular Catalysis A: Chemical 422 (2016) 175-187] reported that [Ru^{III}(DMF)₆](OTf)₃ can react with dipicolylamine in aqueous solution, and this reduced Ru(III) to Ru(II). [Ru(2,2′-dipicolylamine)(OH₂)₃](OTf)₂ is stable to 150° C.

Lonnon et al. [Rhodium, palladium and platinum complexes of tris(pyridylalkyl)amine and tris(benzimidazolylmethyl)amine N4-tripodal ligands, Dalton Trans., 2006, 3785-3797, DOI: 10.1039/b602556k] prepared crystal structures of Rh, Pd, and Pt with tris(2-pyridylmethyl)amine.

Song et al. [Cadmium(II) complexes containing NO-substituted N,N-di(2-picolyl)amine: The formation of monomeric versus dimeric complexes is affected by the N′-substitution group on the amine moiety, J. Organometallic Chem. 783 (2015) 55e63, doi.org/10.1016/j.jorganchem.2015.02.011] showed that N-alkyldipicolylamine derivatives tend to form dimeric complexes with Cd²⁺.

Thus, it is contemplated that the polymer compounds of the present disclosure comprising heterocyclic chelators such as DPA and bis((1H-imidazol-2-yl-)methyl)amine can be used to chelate many different soft metals and open a number of new mass channels for mass cytometry applications based on the stable isotopes of the metals. A non-limiting summary is shown in Table 1 below.

TABLE 1
Exemplary Metals and Corresponding Mass Channels

| | Stable isotopes | |
|---|---|---|
| metal | (m/z) | Comments about the polymer or complex |
| Re²⁺ | 185, 187 | Synthesized, characterized, tested by MC |
| Pt²⁺ | 190, 192, 194, | Synthesized, characterized, tested by MC |
| | 195, 196, 198 | |
| Pd²⁺ | 102, 104, 105, | Synthesized, characterized by NMR |
| | 106, 108, 110 | |
| Nb³⁺ | 93 | known to bind DPA |
| Tc | 99 | known to bind DPA |
| Hg²⁺ | 198, 199, 200, | Forms 1:1 or 1:2 complex with DPA |
| | 201, 202 | |
| Ag⁺ | 107, 109 | Synthesized, characterized by NMR |
| Au³⁺ | 197 | Computationally predicted to bind to DPA |
| Mo²⁺ | 92, 94, 95, | Forms 1:1 complex with DPA |
| | 96, 97, 98 | |
| Ru²⁺ | 96, 98, 99, 100, | Forms 1:1 complex with DPA |
| | 101, 102, 104 | |
| Rh | 103 | Forms an octahedral complex with |
| | | tris(2-pyridylmethyl)-amine |
| Cd²⁺ | 106, 108, 110, | Tends to form dimeric complexes with |
| | 111, 112, 114 | dipic. These have low solubility. |

In some embodiments, M is a soft metal. For example, M can be selected from Re, Pt, Pd, Nb, Tc, Hg, Ag, Au, Mo, Ru, Rh, Cd, W, Os, or mixtures thereof.

In some embodiments, M is non-radioactive. It is contemplated that when the compounds of the present disclosure can be used in a radiometric detection assay. As such, when the compounds are used in a radiometric detection assay, M can be radioactive.

In some embodiments, M is isotopically enriched. For example, M does not comprise a naturally occurring mixture of isotopes.

In another aspect, the present disclosure includes a compound of Formula II as defined herein for use in mass cytometry.

In another aspect, the present disclosure includes an element tag comprising a linear or a branched polymer comprising a plurality of chelating groups, wherein at least one chelating group is chelated to a soft metal atom of the soft metal, the soft metal being a single isotope.

In some embodiments, the element tag is a compound of the present disclosure.

In another aspect, the present disclosure includes a kit comprising
an isotopic composition comprising multiple soft metal atoms of a single isotope of a soft metal, and
an element tag comprising a linear or branched polymer comprising a plurality of chelating groups comprising two nitrogen-containing 5-membered or 6-membered heterocycles, wherein each chelating group includes at least one soft metal atom of the isotopic composition or is capable of binding at least one soft metal atom of the isotopic composition.

In some embodiments, the kit does not comprise any radioactive soft metal.

In some embodiments, the isotopic composition does not comprise a natural mixture of isotopes.

It is contemplated that the element tag can be functionalised to bind a biomolecule. For example, the element tag can be covalently attached to a biomolecule.

In some embodiments, the kit further comprises a biomolecule.

For example, the biomolecule can be an oligonucleotide. For example, the biomolecule can be an antibody or other affinity reagent.

In some instances, each chelating group includes at least one soft metal atom of the isotopic composition.

In some embodiments, the isotopic composition is a soft metal solution provided separate from the element tag, and wherein each chelating group is capable of binding at least one soft metal atom of the isotopic composition.

In some embodiments, the kit further comprises an additional isotopic composition. For example, the additional isotopic composition comprises multiple additional soft metal atoms of an additional single isotope of a soft metal that is different from the single isotope of the soft metal of the isotopic composition.

In some embodiments, the kit further comprises an additional element tag comprising an additional linear or branched polymer comprising a plurality of additional chelating groups.

In some aspects, each chelating group of the linear or branched polymer of the element tag includes at least one soft metal atom of the isotopic composition, and wherein each additional chelating group of the additional linear or branched polymer of the additional element tag includes at least one additional soft metal atom of the additional isotopic composition.

In some embodiments, each element tag is covalently bound to a different antibody.

In an embodiment, each chelating group is capable of binding at least one soft metal atom of the isotopic composition, and each chelating group is selected from dipicolylamine or bis((1H-imidazol-2-yl)methyl)amine, wherein each imidazole is optionally substituted with one or more polar functional groups selected from C1-C6 COOH, C1-C6 alkoxy, C1-C6 alkyl phosphonate, alkyl ether, polyether, or combinations thereof.

In some embodiments, the kit further comprising a reagent for covalent attachment of the element tag to an antibody.

In some embodiments, each element tag is independently a compound of Formula I as described herein or a compound of Formula II as described herein.

The kits described in the above embodiments may have any additional aspects described herein, such as an element tag comprising one or more solubility modifiers (e.g., on the same pendant group as the chelating group).

### III. Methods and Uses

In another aspect, the present disclosure includes a method comprising:
providing an isotopic composition comprising multiple soft metal atoms of a single isotope of a soft metal;
providing an element tag comprising a linear or branched polymer comprising a plurality of chelating groups each independently comprising two nitrogen-containing 5-membered or or 6-membered heterocycles, wherein each chelating group is capable of binding at least one of the soft metal atom of the isotopic composition; and
binding the soft metal atoms of the isotopic composition to the one or more chelating groups of the element tag.

In some embodiments, the soft metal atoms are non-radioactive.

In some embodiments, the isotopic composition does not comprise a natural mixture of isotopes.

In some embodiment, the method can further comprise providing an additional isotopic composition wherein the additional isotopic composition comprises multiple additional soft metal atoms of an additional single isotope of a non-radioactive soft metal that is different from the single isotope of the non-radioactive soft metal of the isotopic composition.

For example, the method further comprises providing an additional element tag comprising an additional linear or branched polymer comprising a plurality of chelating groups.

In some embodiments, each chelating group of the linear or branched polymer of the element tag includes at least one soft metal atom of the isotopic composition, and wherein each additional chelating group of the additional linear or branched polymer of the additional element tag includes at least one additional soft metal atom of the additional isotropic composition.

In some embodiments, the method further comprises:
providing a biomolecule; and
covalently binding the biomolecule to the element tag.

In another aspect, the present disclosure includes a method for the analysis of an analyte in a biological sample, comprising:
(i) incubating an element tagged affinity reagent with the analyte, the element tagged affinity reagent comprising an affinity reagent tagged with an element tag, the element tag comprising a linear or branched polymer having multiple chelating groups each independently comprising two nitrogen-containing 5-membered or 6-membered heterocycles, the element tag further comprising multiple soft metal atoms of a single isotope of a soft metal;
wherein:
   each chelating group of the element tag includes at least one of the soft metal atoms or is capable of binding at least one of the soft metal atoms, and the affinity reagent specifically binds the analyte,
(ii) separating unbound element tagged affinity reagent from bound element tagged affinity reagent; and
(iii) analyzing the element tag bound to the affinity reagent attached to the analyte by mass spectrometric atomic spectroscopy.

In some embodiments, the soft metal atoms are non-radioactive.

In some embodiments, the soft metal does not comprise a natural mixture of isotopes.

In some embodiments, incubating the element tagged affinity reagent with the analyte comprises:
incubating two or more differential element tagged affinity reagents with two or more analytes, wherein the element tagged affinity reagents specifically bind with the two or more analytes to produce two or more differentially tagged analytes, wherein analyzing the element tag bound to the affinity reagent comprises analyzing the differential element tags bound to the two or more analytes by mass spectrometric atomic spectroscopy.

In some instances, the affinity reagent is further labeled with a fluorescent label.

In some embodiments, the mass spectrometric atomic spectroscopy is ICP-MS. In an embodiment, the mass spectrometric atomic spectroscopy is by a mass spectrometer based flow cytometer.

In some embodiments, the affinity reagent is an antibody.

In some embodiments, the affinity reagent specifically binds biotin.

In some embodiments, the affinity reagent is an oligonucleotide.

In some embodiments, the element tagged affinity reagent is configured to bind with an analyte in a biological sample, and the biological sample comprises cells. In some embodiments, the element tagged affinity reagent is configured to bind with an analyte in a biological sample, and the soft metal is an element that does not naturally occur in the biological sample.

In some embodiments, the soft metal is selected from Re, Pt, Pd, Nb, Tc, Hg, Ag, Au, Mo, Ru, Rh, Cd, W, Os, or mixtures thereof.

In some embodiments, the element tag is a compound of Formula I as described herein, or a compound of Formula II as described herein.

The methods of the above embodiments may have any additional aspects described herein, such as an element tag comprising one or more solubility modifiers (e.g., on the same pendant group as the chelating group).

The present disclosure also provides the following embodiments:

Embodiment 1. A Compound of Formula I

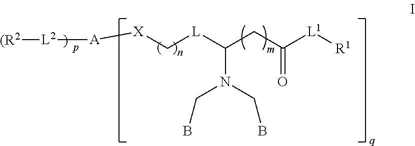

wherein
A is a polymer backbone, optionally, the polymer is a linear polymer, branched polymer, hyperbranched polymer, co-polymer, or combinations thereof;
each B is independently a nitrogen-containing 5-membered to 7-membered heterocycle, optionally substituted with one or more polar functional groups selected from C1-C6 COOH, C1-C6 alkoxy, C1-C6 alkyl phosphonate, alkyl ether, polyether, or combinations thereof; and
L is absent or a linker;
each L² is independently absent or a linker;
each R² is independently a first modifying group selected from a solubility modifier, a reactive functional group, a biomolecule, or combinations thereof;
X is a functional group selected from ester, ether and amide;
each L¹ is independently absent or a linker;
each R¹ is independently H, C1 to C8 alkyl, C2 to C8 alkenyl, C3-C8 cycloalkyl, OH, C1 to C10 alkoxy, C1 to C10 alkyl amine, a solubility modifier, a reactive functional group, a biomolecule and combinations thereof;
n is an integer from 0 to 7;
m is an integer from 0 to 4;
p is an integer from 0 to 3; and
q is an integer above 0.

Embodiment 2. The compound of embodiment 1, wherein each B is independently a nitrogen-containing 5-membered or 6-membered heteroaryl, optionally substituted with one or more polar functional groups selected from COOH, C1-C6 alkoxy, C1-C6 alkyl phosphonate, alkyl ether, polyether, or combinations thereof, and wherein optionally one or more B are coordinated to a soft metal, and/or conjugated to one or more biomolecules.

Embodiment 3. The compound of embodiment 1 or 2, wherein each B is independently pyridine or imidazole, optionally substituted with one or more polar functional groups selected from COOH, C1-C6 alkoxy, C1-C6 alkyl phosphonate, alkyl ether, polyether, or combinations thereof, and wherein optionally one or more B are coordinated to a soft metal, and/or conjugated to one or more biomolecules.

Embodiment 4. The compound of any one of embodiments 1 to 3, wherein one or more B are coordinated to a soft metal.

Embodiment 5. The compound of any one of embodiments 1 to 4, wherein R¹ or R² is the biomolecule, optionally the biomolecule is an affinity reagent, such as an antibody.

Embodiment 6. The compound of any one of embodiments 1 to 5, wherein X is amide.

Embodiment 7. The compound of embodiment 6, wherein X is —C(O)NR⁴— or —NR⁴C(O)—, wherein R⁴ is H or C1 to C4 alkyl.

Embodiment 8. The compound of any one of embodiments 1 to 7, wherein X is —C(O)NR⁴— and the compound has a structure of Formula Ia

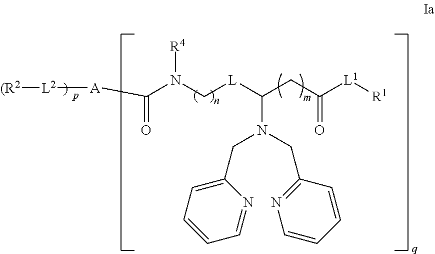

Embodiment 9. The compound of any one of embodiments 1 to 7, wherein X is —NR⁴C(O)— and the compound has a structure of Formula Ib

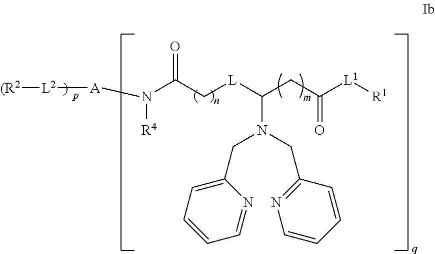

Embodiment 10. The compound of any one of embodiments 1 to 7, wherein X is —C(O)NR⁴— and the compound has a structure of Formula Ic

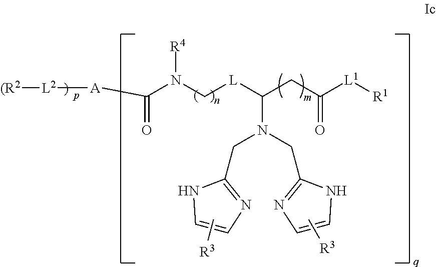

wherein each R³ is independently selected from H, C1 to C5 alkyl, C2 to C5 alkenyl, C1-C6 COOH, C1-C6 alkoxy, C1-C6 alkyl phosphonate, alkyl ether, or polyether.

Embodiment 11. The compound of embodiment 10, wherein the compound has a structure of Formula Id or Ie

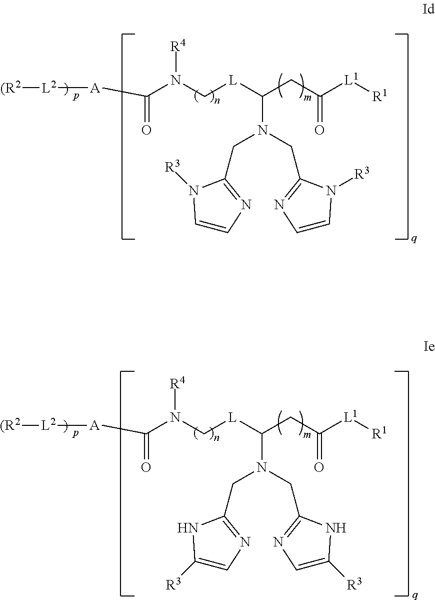

Embodiment 12. The compound of any one of embodiments 1 to 7, wherein X is —NR⁴C(O)— and the compound has a structure of Formula If

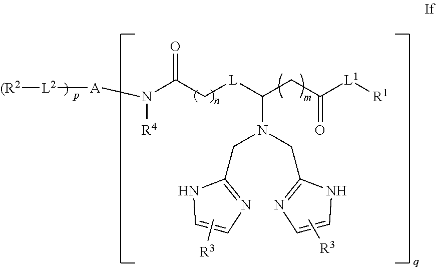

wherein each R³ is independently selected from H, C1 to C5 alkyl, C2 to C5 alkenyl, C1-C6 COOH, C1-C6 alkoxy, C1-C6 alkyl phosphonate, alkyl ether, or polyether.

Embodiment 13. The compound of embodiment 12, wherein the compound has a structure of Formula Ig or Ih

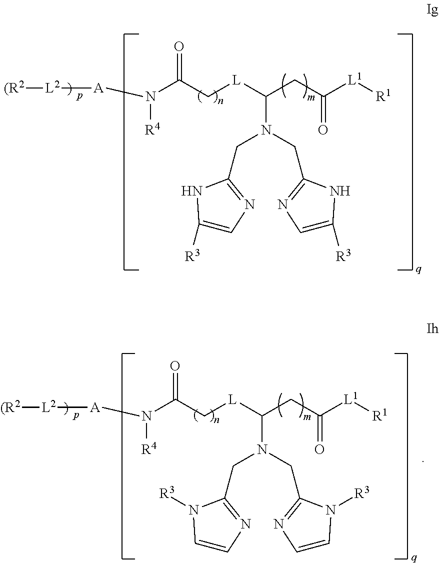

Embodiment 14. The compound of any one of embodiments 10 to 13, wherein each R³ is independently selected from H, —(CH₂)₁₋₃COOH, —(CH₂)₁₋₃O(CH₂)₁₋₂CH₃, —(CH₂)₂₋₄OH, —(CH₂)₂₋₅P(O)(OCH₂CH₃)₂, or —CH₂CH(OMe)₂.

Embodiment 15. The compound of any one of embodiments 1 to 14, wherein n is 2, 3, 4, or 5.

Embodiment 16. The compound of any one of embodiments 1 to 15, wherein m is 0, 1, or 2.

Embodiment 17. The compound of any one of embodiments 1 to 16, wherein p is 1 or 2.

Embodiment 18. The compound of any one of embodiments 1 to 17, wherein A is selected from polyacrylate, polyacrylamide, polyether, polyamino acid, polyvinyl amine, poly(2-oxazoline), polyethylene glycol, polysaccharide, dendrimer, co-polymers thereof, or combinations thereof.

Embodiment 19. The compound of embodiment 18, wherein A is polyamino acid.

Embodiment 20. The compound of embodiment 18 or 19, wherein the polyamino acid is polyglutamic acid, polyaspartic acid, polylysine, poly(2,4-dimethylaminobutyric acid) (polyDab), poly(2,4-diaminopimelic acid) (polyDap), derivatives thereof, or combinations thereof.

Embodiment 21. The compound of any one of embodiments 1 to 20, wherein each linker independently comprises or is independently selected from C3-C8 alkyl amine, C3-C8 alkylene, C3-C8 cycloalkyl, C3-C8 heterocycloalkyl, 5-membered or 6-membered aryl or heteroaryl, alkylaryl, alkylheteroaryl, C3-C8 cycloalkylaryl, C3-C8 cycloalkylheteroaryl, C(O), C(O)O, amide, amine, thioether, maleimide-thiol conjugate, polyethylene glycol (PEG), or mixtures thereof, optionally each of the amine, alkylene, aryl, alkylaryl, alkylheteroaryl, cycloalkyl, cycloalkylaryl, and cycloalkylheteroaryl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

Embodiment 22. The compound of any one of embodiments 1 to 21, wherein each L² independently comprises or is independently selected from C3-C8 alkylene, C3-C8 alkyl amine, ester, amine, amide, thioether, maleimide-thiol conjugate, PEG, or mixtures thereof, optionally each of the alkylene and alkyl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

Embodiment 23. The compound of any one of embodiments 1 to 22, wherein each L¹ independently comprises or is independently selected from C3-C8 alkylene, C3-C8 alkyl amine, ester, amine, amide, thioether, maleimide-thiol conjugate, PEG, or mixtures thereof, optionally each of the alkylene and alkyl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

Embodiment 24. The compound of any one of embodiments 1 to 23, wherein L is absent or a C3-C8 alkyl amine.

Embodiment 25. The compound of any one of embodiments 1 to 24, wherein the solubility modifier of the first modifying group of each R² and the solubility modifier of the second modifying group each independently comprises polyethyleneglycol (PEG), sugar, oligosaccharide, or zwitterionic polymer such as poly(carboxylbetaine) methacrylate or poly(sulfobetaine) methacrylate (PBSMA).

Embodiment 26. The compound of any one of embodiments 1 to 25, wherein the reactive functional group is for attachment to one or more biomolecules.

Embodiment 27. The compound of any one of embodiments 1 to 26, wherein the reactive functional group of the first modifying group of each R² and the reactive functional group of the second modifying group is each independently selected from carboxylic acid, maleimide, thiol, azide, dibenzocyclooctyne (DBCO), trans-cyclooctene (TCO), tetrazine, furan, or aldehyde.

Embodiment 28. The compound of any one of embodiments 1 to 27, wherein the one or more biomolecules are each independently selected from a small molecule, a polypeptide, an oligonucleotide, a lipid, a carbohydrate, or a mixture thereof.

Embodiment 29. The compound of embodiment 28, wherein the one or more biomolecules are each independently an affinity reagent, optionally, wherein the affinity reagent is an antibody.

Embodiment 30. The compound of embodiment 1, wherein the compound is selected from

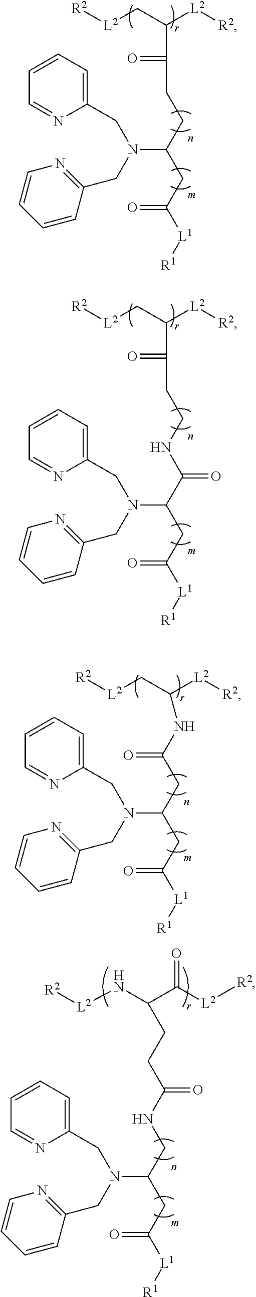

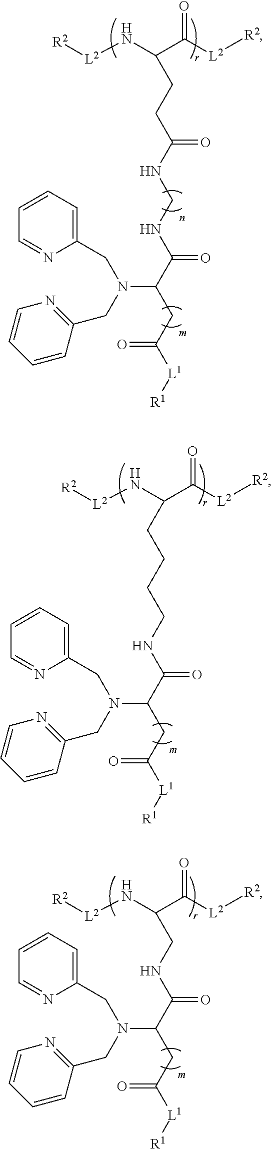

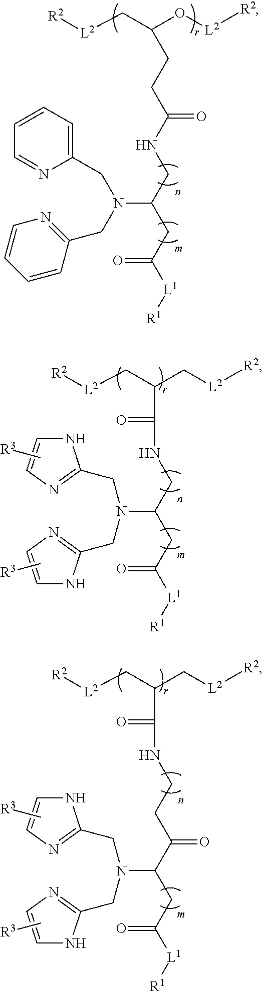

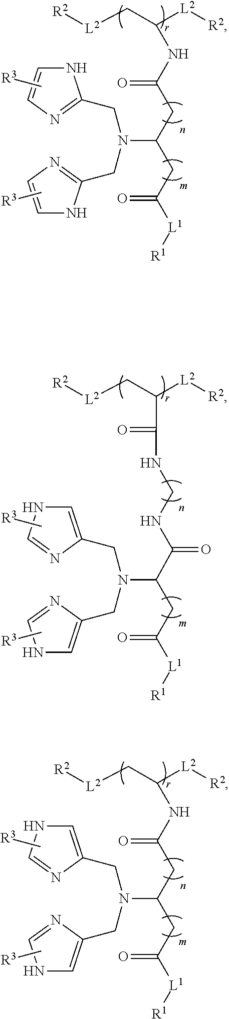

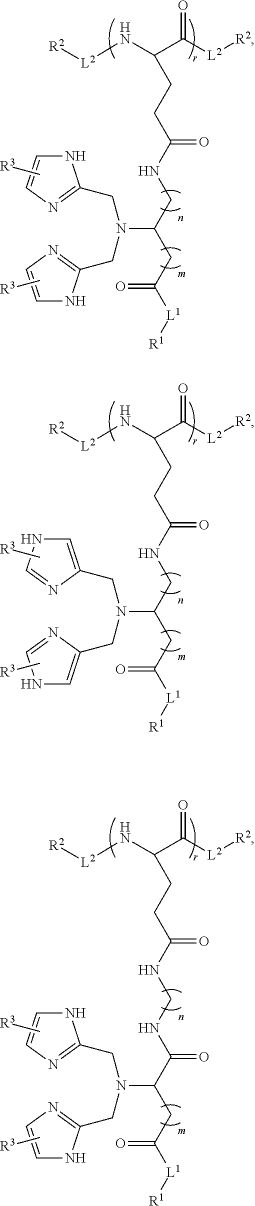

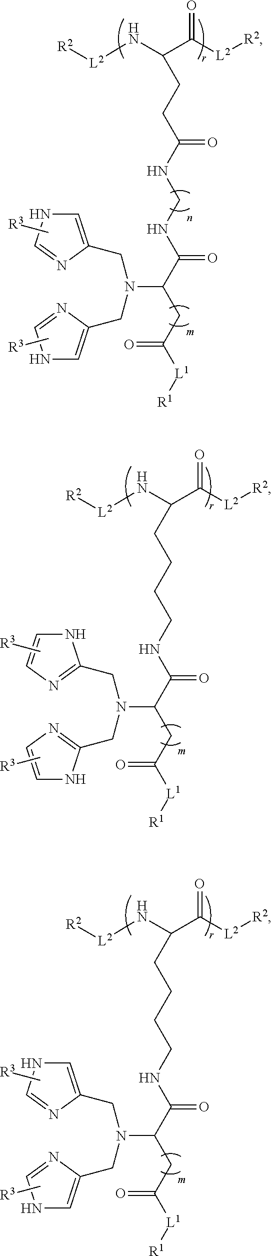

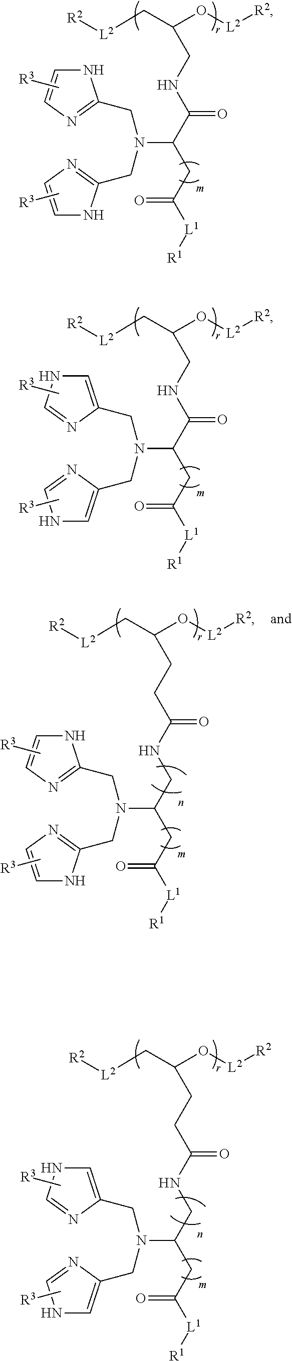

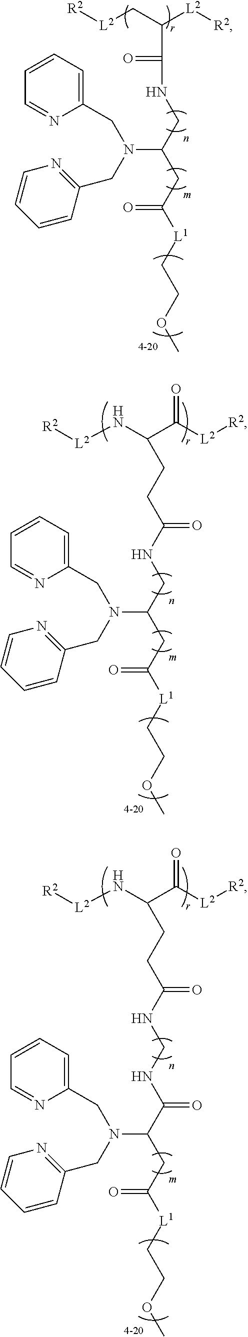

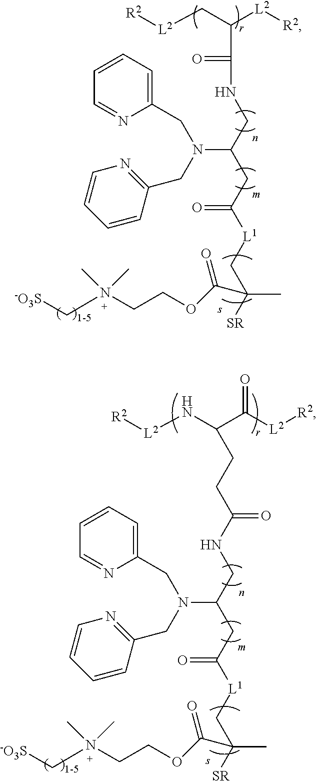

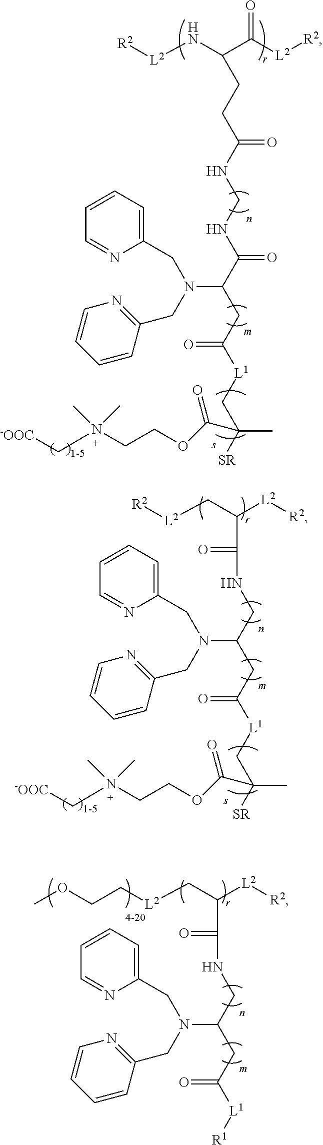

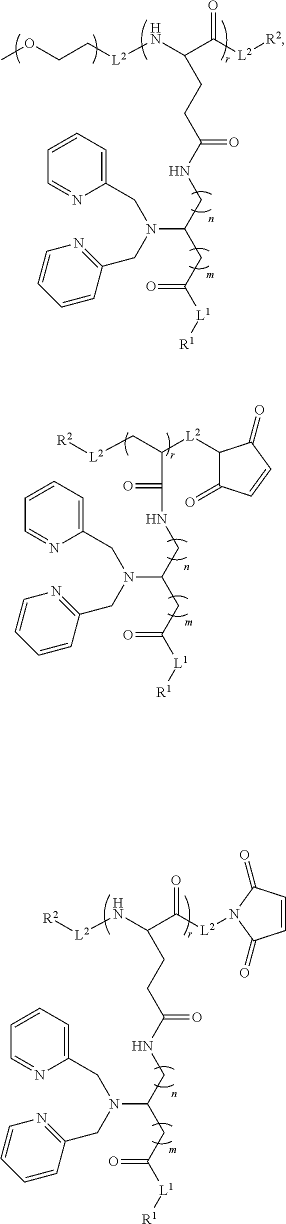

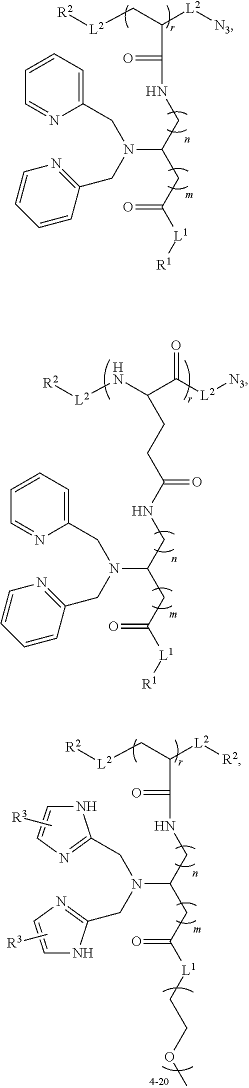

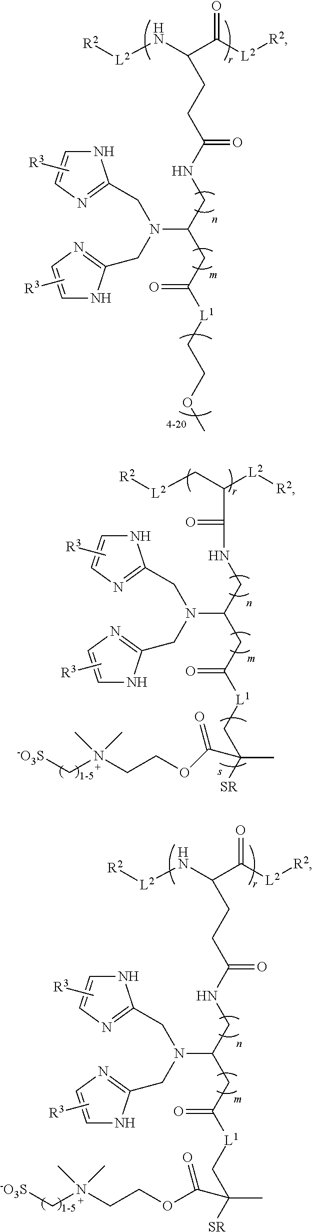

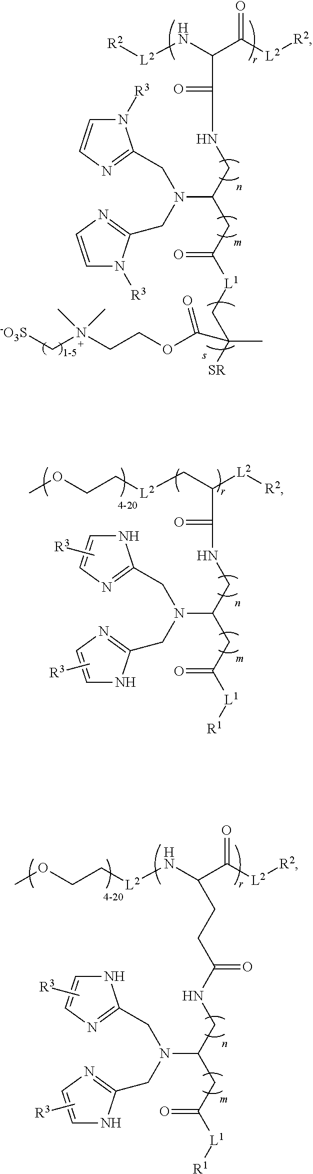

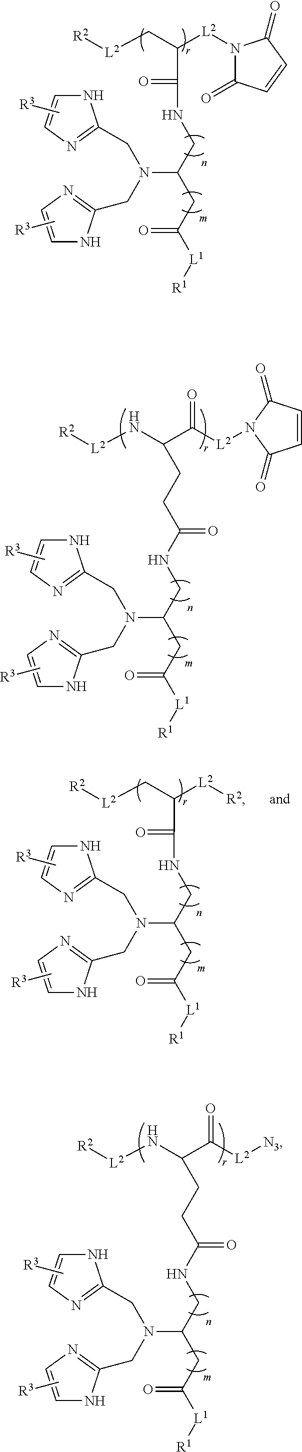

wherein r is about 3 to about 200, about 6 to about 30, or about 10 to about 25, and wherein R¹, R², L¹, L² and R³ are each as defined in any one of embodiments 10 to 14.

Embodiment 31. The compound of embodiment 1, wherein the compound is selected from

wherein s is about 1 to about 50, about 2 to about 40, about 5 to about 30, about 10 to about 30, about 5 to about 35, or about 20 to about 30, and r is about 3 to about 200, about 6 to about 30, or about 10 to about 25, and wherein R¹, R², L¹, L² and R³ are each as defined in any one of embodiments 10 to 14.

Embodiment 32. The compound of embodiment 1, wherein the compound is selected from

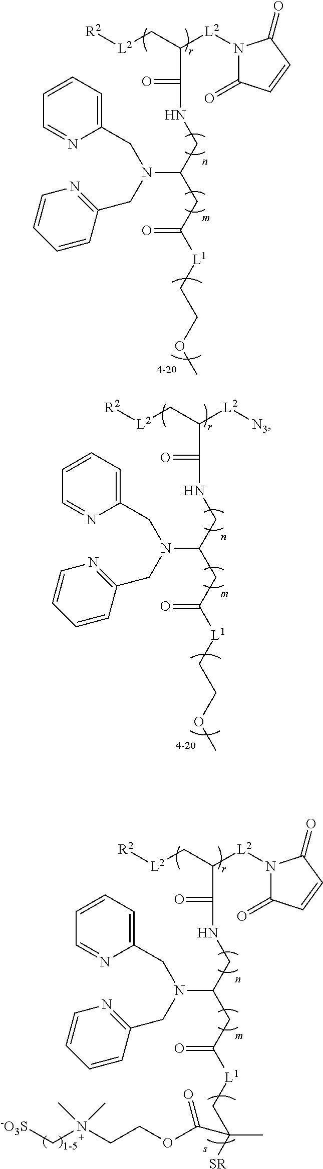

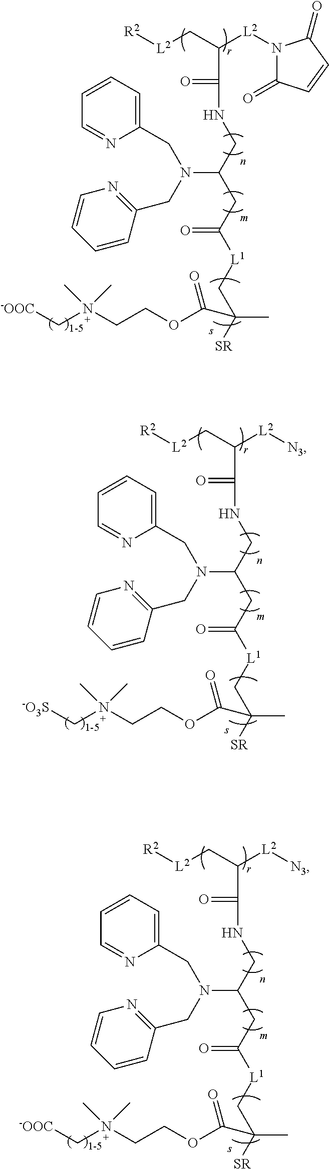

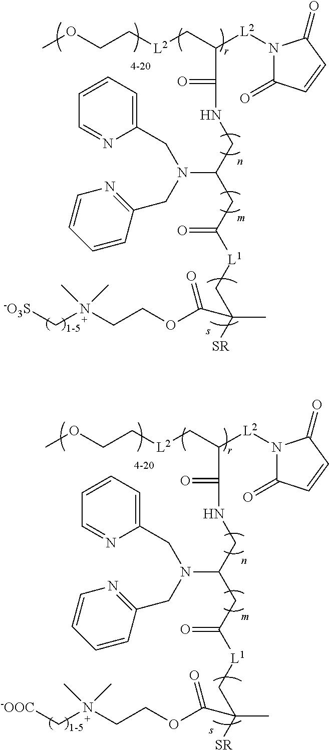

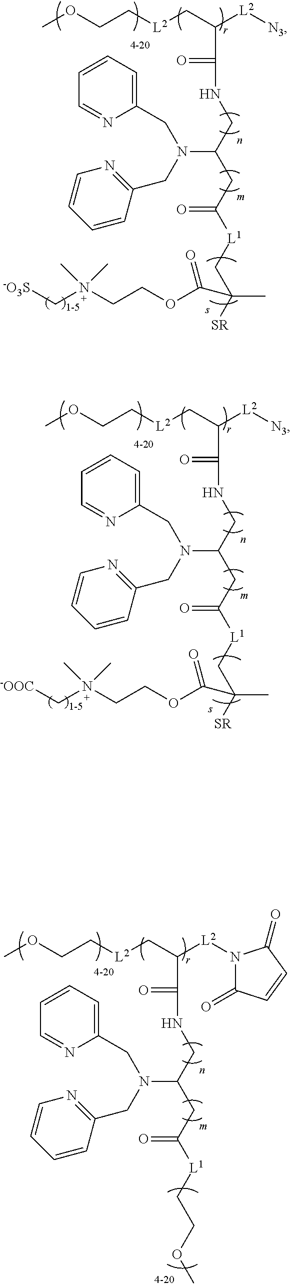

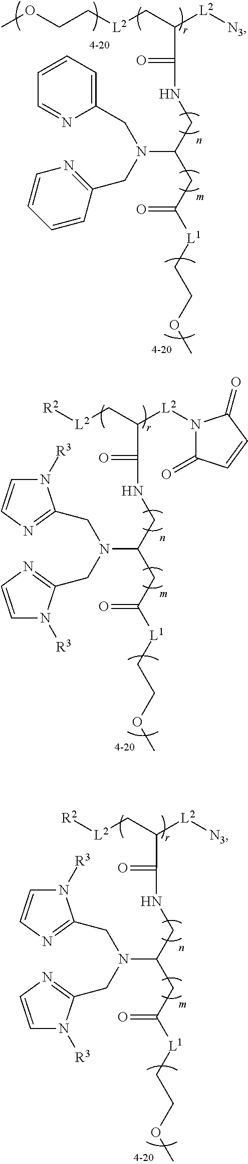

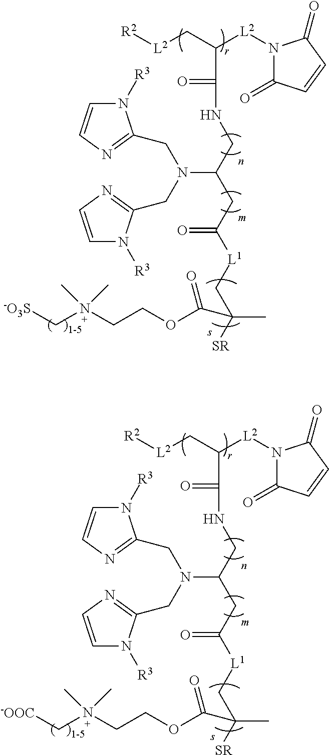

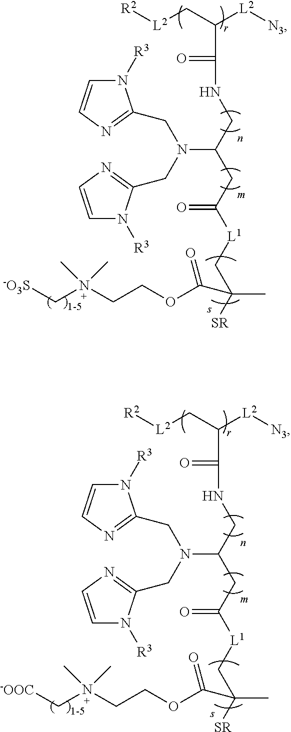

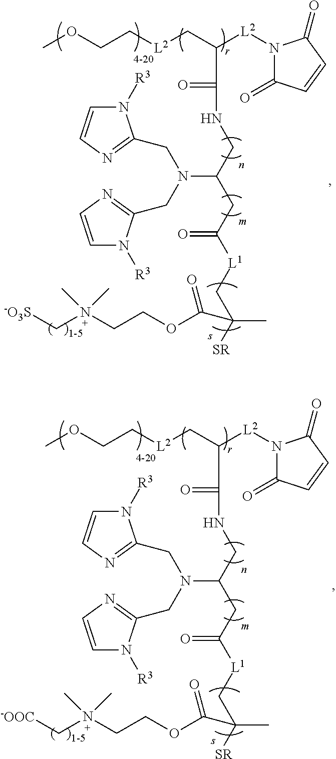

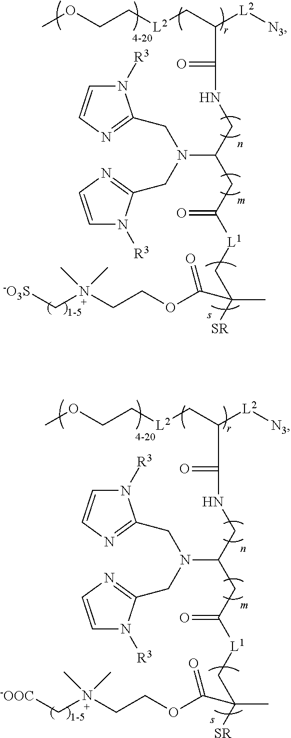

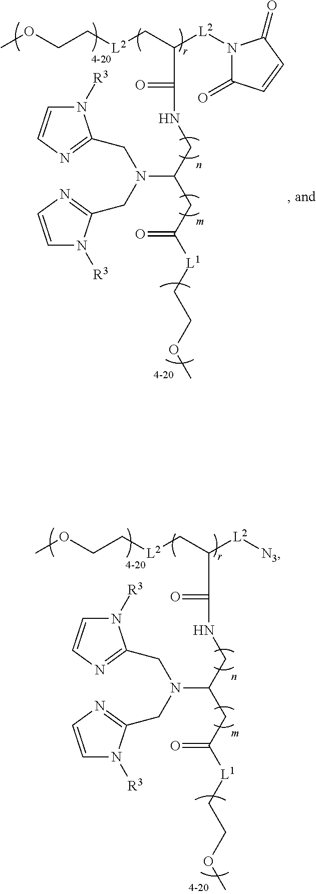

wherein s is about 1 to about 50, about 2 to about 40, about 5 to about 30, about 10 to about 30, about 5 to about 35, or about 20 to about 30, and r is about 3 to about 200, about 6 to about 30, or about 10 to about 25, and wherein R¹, R², L¹, L² and R³ are each as defined in any one of embodiments 10 to 14.

Embodiment 33. The compound of embodiment 1, wherein the compound is selected from

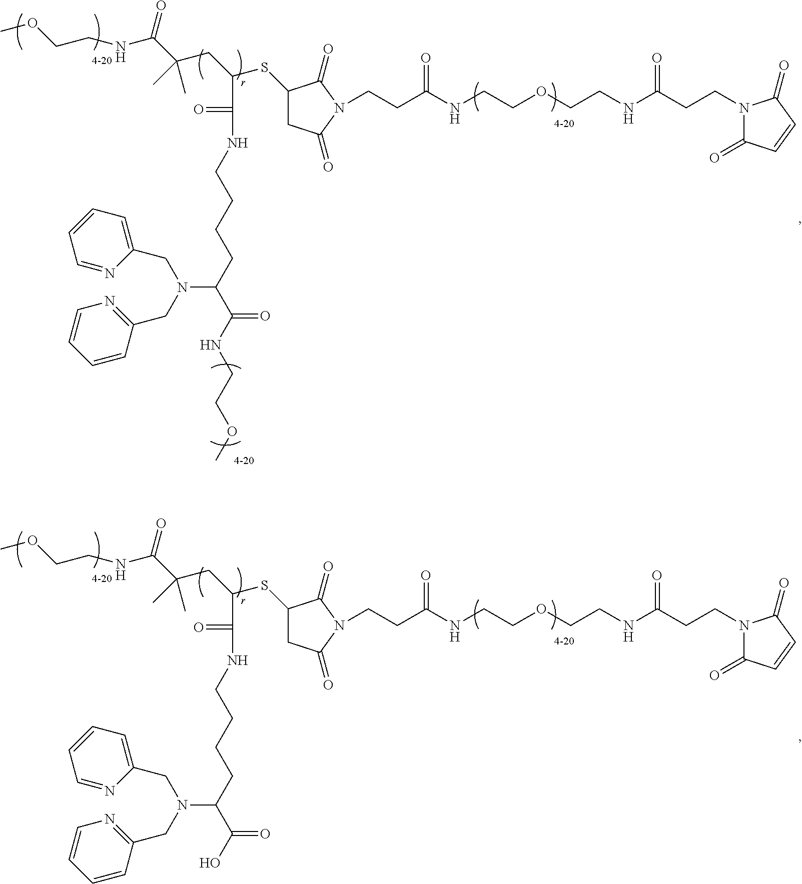

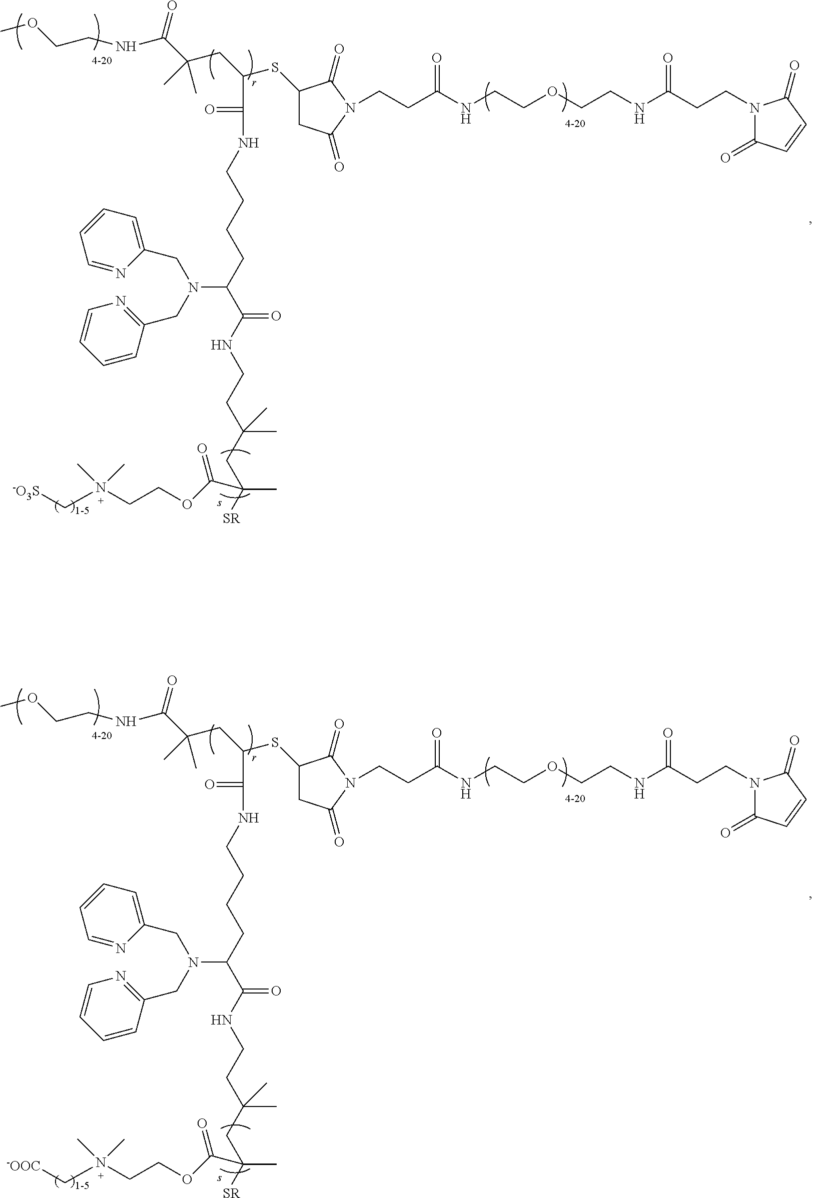

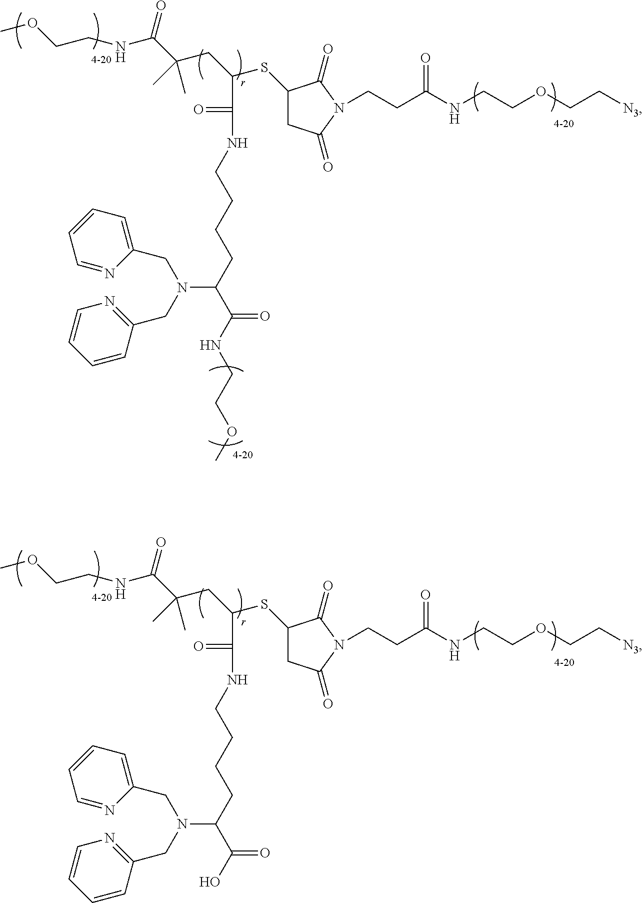

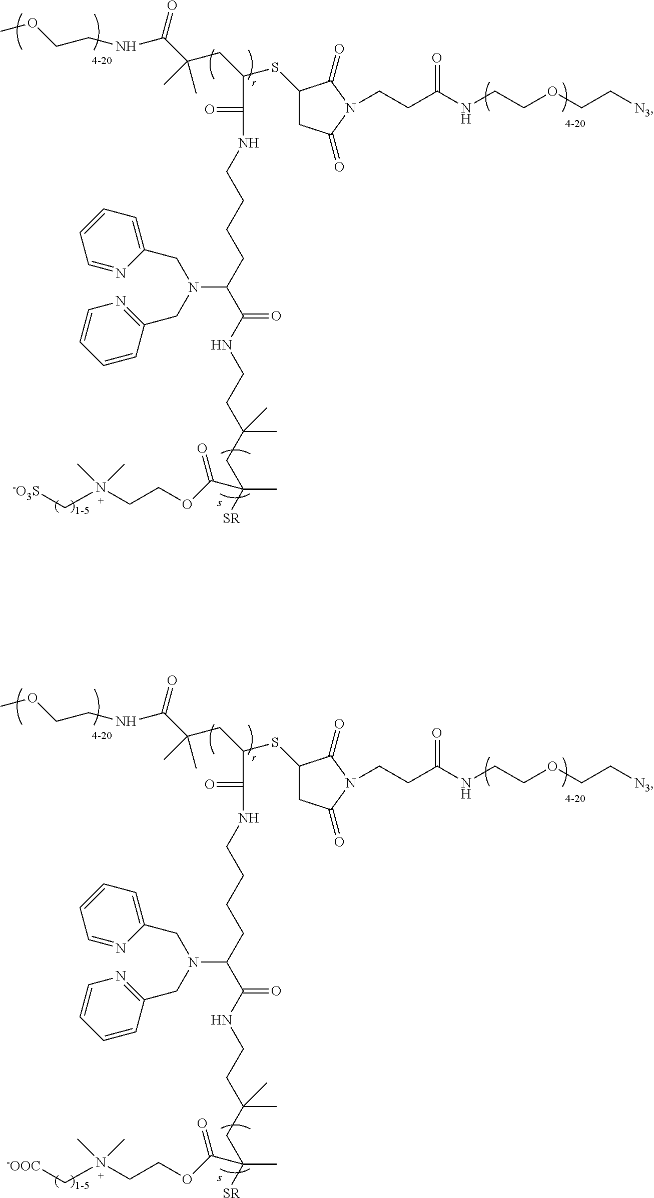

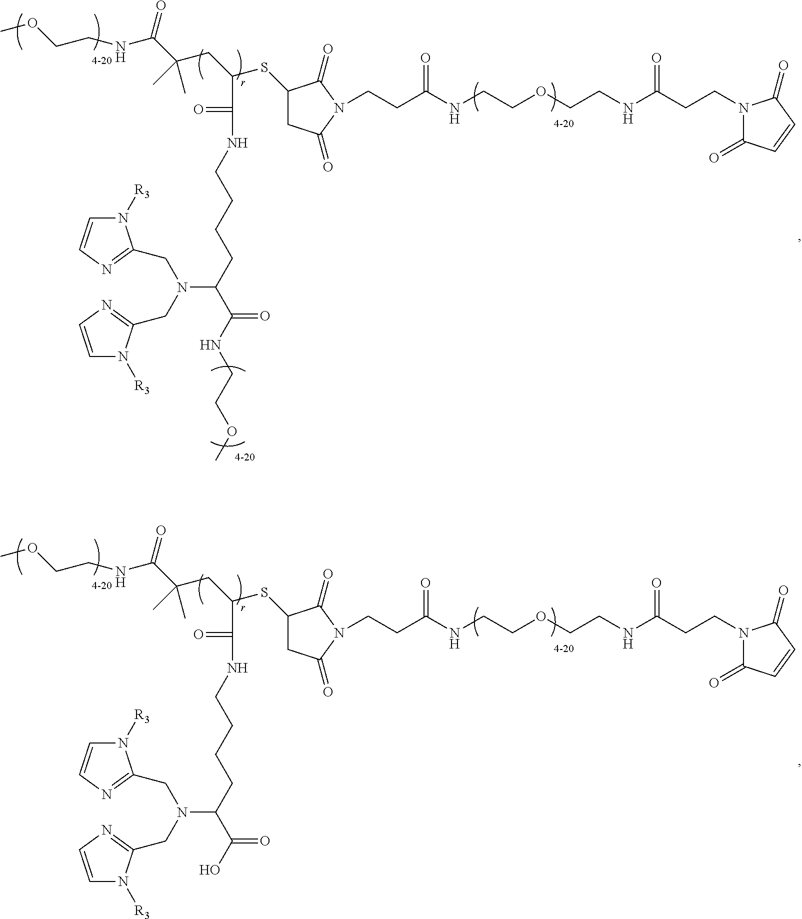

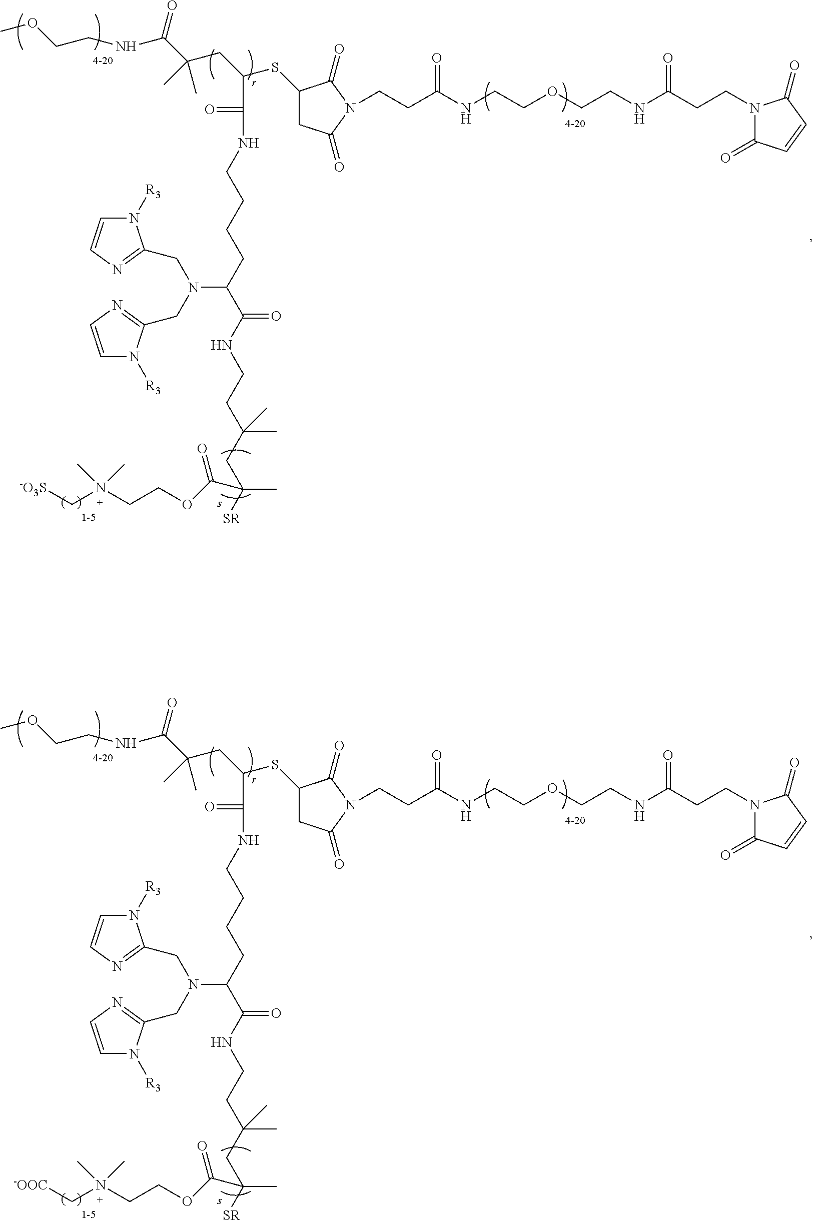

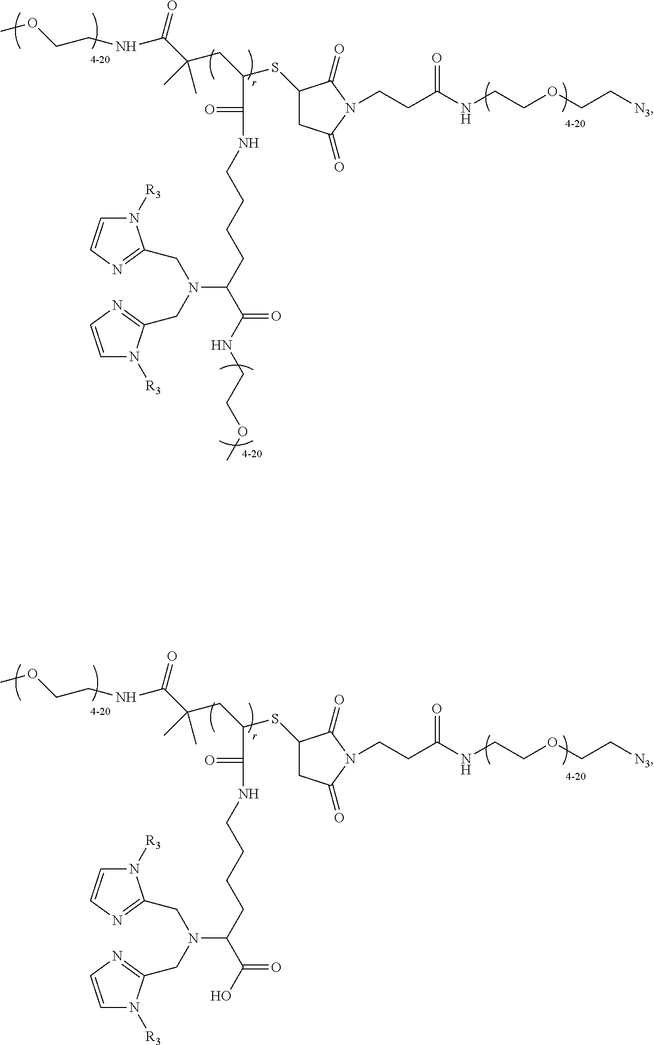

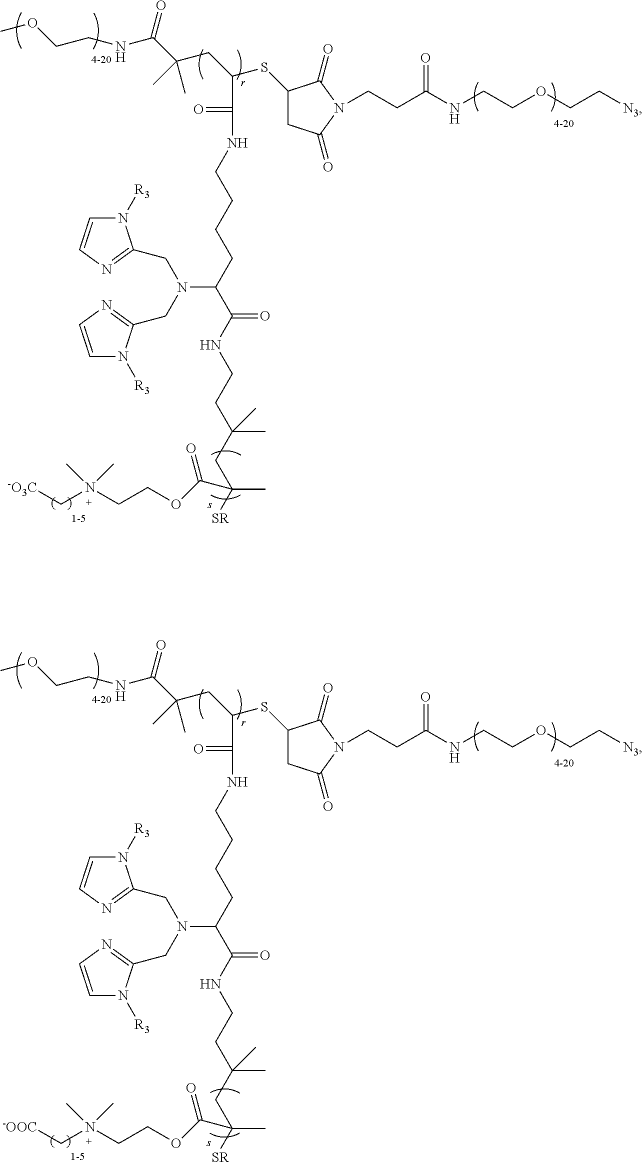

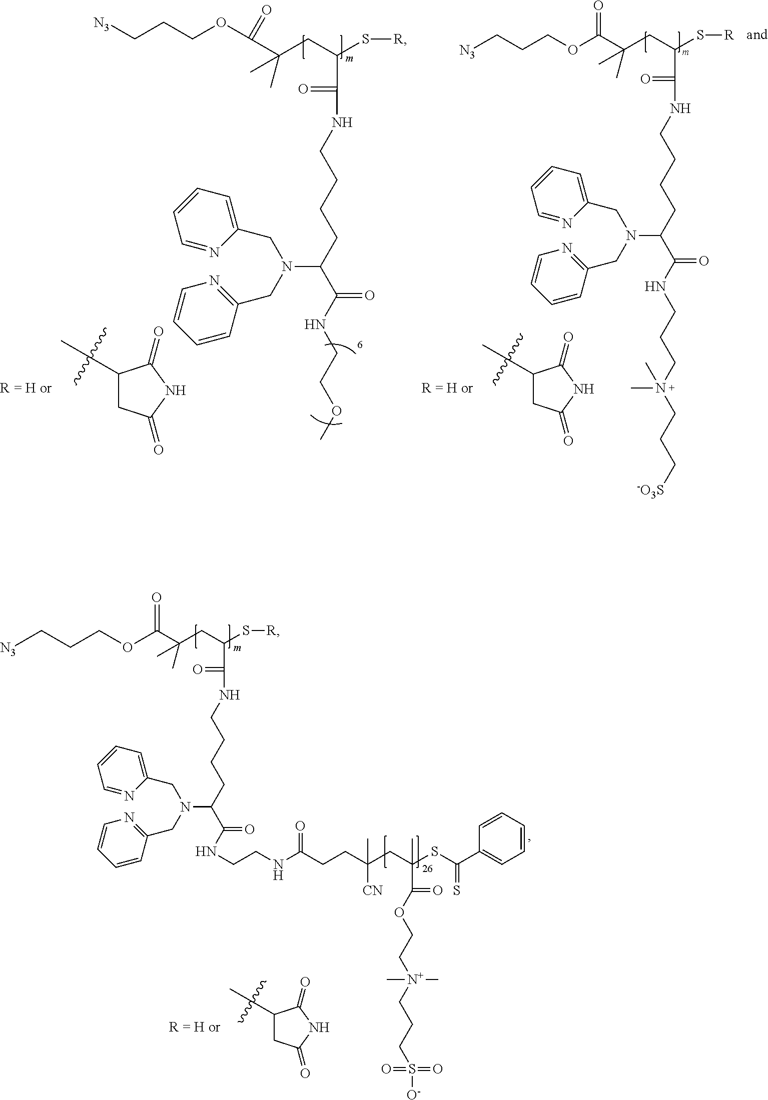

wherein s is about 1 to about 50, about 2 to about 40, about 5 to about 30, about 10 to about 30, about 5 to about 35, or about 20 to about 30, and r is about 3 to about 200, about 6 to about 30, or about 10 to about 25, and wherein R³ is as defined in any one of embodiments 10 to 14.

Embodiment 34. A compound of Formula I as defined in any one of embodiments 1 to 33, wherein the compound of Formula I is chelated to one or more metal M, and wherein the compound has a structure of Formula II

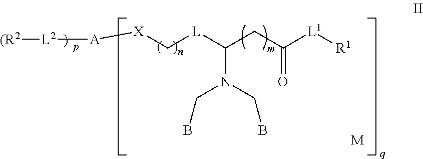

or a derivative or salt thereof.

Embodiment 35. The compound of embodiment 34, wherein M is a soft metal.

Embodiment 36. The compound of embodiment 34 or 35, wherein M is selected from Re, Pt, Pd, Nb, Tc, Hg, Ag, Au, Mo, Ru, Rh, Cd, W, Os, or mixtures thereof.

Embodiment 37. The compound of any one of embodiments 34 to 36, or the composition of any one of embodiments 35 to 37, wherein M is non-radioactive.

Embodiment 38. The compound of any one of embodiments 34 to 37, or the composition of any one of embodiments 35 to 38, wherein M is isotopically enriched.

Embodiment 39. A composition comprising one or more compounds of Formula I, each independently as defined in any one of embodiments 1 to 33 or one or more compounds of Formula II, each independently as defined in any one of embodiments 34 to 38, and a solvent.

Embodiment 40. A compound of Formula I as defined in any one of embodiments 1 to 33 or a compound of Formula II as defined in any one of embodiments 34 to 38 for use in mass cytometry.

Embodiment 41. An element tag comprising a linear or a branched polymer comprising a plurality of chelating groups, wherein each chelating group is capable of binding a soft metal, the soft metal being a single isotope, and wherein at least one chelating group is chelated to a soft metal atom of the soft metal.

Embodiment 42. A kit comprising an isotopic composition comprising multiple soft metal atoms of a single isotope of a soft metal; and an element tag comprising a linear or branched polymer comprising a plurality of chelating groups comprising two nitrogen-containing 5-membered or 6-membered heterocycles, wherein each chelating group of the element tag includes at least one soft metal atom of the isotopic composition or is capable of binding at least one soft metal atom of the isotopic composition; optionally wherein the kit does not comprise any radioactive soft metal.

Embodiment 43. The kit of embodiment 42, wherein the isotopic composition does not comprise a natural mixture of isotopes.

Embodiment 44. The kit of embodiment 42 or 43, wherein the element tag is functionalised to bind a biomolecule.

Embodiment 45. The kit of embodiment 42 or 43, wherein the element tag is covalently attached to a biomolecule.

Embodiment 46. The kit of any one of embodiments 42 to 44 further comprising a biomolecule.

Embodiment 47. The kit of any one of embodiments 43 to 46, wherein the biomolecule is an oligonucleotide.

Embodiment 48. The kit of any one of embodiments 43 to 46, wherein the biomolecule is an antibody.

Embodiment 49. The kit of any one of embodiments 42 to 48, wherein each chelating group includes at least one soft metal atom of the isotopic composition.

Embodiment 50. The kit of any one of embodiments 42 to 48, wherein the isotopic composition is a soft metal solution provided separate from the element tag, and wherein each chelating group is capable of binding at least one soft metal atom of the isotopic composition.

Embodiment 51. The kit of any one of embodiments 42 to 50 further comprising an additional isotopic composition, wherein the additional isotopic composition comprises multiple additional soft metal atoms of an additional single isotope of a soft metal that is different from the single isotope of the soft metal of the isotopic composition.

Embodiment 52. The kit of embodiment 51, further comprising an additional element tag comprising an additional linear or branched polymer comprising a plurality of additional chelating groups.

Embodiment 53. The kit of embodiment 52, wherein each chelating group of the linear or branched polymer of the element tag includes at least one soft metal atom of the isotopic composition, and wherein each additional chelating group of the additional linear or branched polymer of the additional element tag includes at least one additional soft metal atom of the additional isotopic composition.

Embodiment 54. The kit of any one of embodiments 42 to 53, wherein each element tag is covalently bound to a different antibody.

Embodiment 55. The kit of embodiment any one of embodiments 42 to 54, wherein each chelating group is capable of binding at least one soft metal atom of the isotopic composition, and each chelating group is selected from dipicolylamine or bis((1H-imidazol-2-yl)methyl)amine, wherein each imidazole is optionally substituted with one or more polar functional groups selected from C1-C6 COOH, C1-C6 alkoxy, C1-C6 alkyl phosphonate, alkyl ether, polyether, or combinations thereof.

Embodiment 56. The kit of any one of embodiments 42 to 55 further comprising a reagent for covalent attachment of the element tag to an antibody.

Embodiment 57. The kit of any one of embodiments 42 to 56, wherein each element tag is independently a compound of Formula I as defined in any one of embodiments 1 to 33 or a compound of Formula II as defined in any one of embodiments 34 to 38.

Embodiment 58. A method comprising:
providing an isotopic composition comprising multiple soft metal atoms of a single isotope of a soft metal;
providing an element tag comprising a linear or branched polymer comprising a plurality of chelating groups each independently comprising two nitrogen-containing 5-membered or 6-membered heterocycles, wherein each chelating group is capable of binding at least one of the soft metal atom of the isotopic composition; and
binding the soft metal atoms of the isotopic composition to the one or more chelating groups of the element tag;
wherein the soft metal atoms are non-radioactive.

Embodiment 59. The method of embodiment 58, wherein the isotopic composition does not comprise a natural mixture of isotopes.

Embodiment 60. The method of embodiment 59 further comprising providing an additional isotopic composition wherein the additional isotopic composition comprises multiple additional soft metal atoms of an additional single isotope of a non-radioactive soft metal that is different from the single isotope of the non-radioactive soft metal of the isotopic composition.

Embodiment 61. The method of any one of embodiments 58 to 60 further comprising providing an additional element tag comprising an additional linear or branched polymer comprising a plurality of chelating groups.

Embodiment 62. The method of any one of embodiments 58 to 61, wherein each chelating group of the linear or branched polymer of the element tag includes at least one soft metal atom of the isotopic composition, and wherein each additional chelating group of the additional linear or branched polymer of the additional element tag includes at least one additional soft metal atom of the additional isotropic composition.

Embodiment 63. The method of any one of embodiments 58 to 62 further comprising:
providing a biomolecule; and
covalently binding the biomolecule to the element tag.

Embodiment 64. A method for the analysis of an analyte in a biological sample, comprising:
(i) incubating an element tagged affinity reagent with the analyte, the element tagged affinity reagent comprising an affinity reagent tagged with an element tag, the element tag comprising a linear or branched polymer having multiple chelating groups each independently comprising two nitrogen-containing 5-membered or 6-membered heterocycles, the element tag further comprising multiple soft metal atoms of a single isotope of a soft metal;
wherein:
   each chelating group of the element tag includes at least one of the soft metal atoms or is capable of binding at least one of the soft metal atoms,
   the soft metal atoms are non-radioactive, and
   the affinity reagent specifically binds the analyte,
(ii) separating unbound element tagged affinity reagent from bound element tagged affinity reagent; and
(iii) analyzing the element tag bound to the affinity reagent attached to the analyte by mass spectrometric atomic spectroscopy.

Embodiment 65. The method of embodiment 64, wherein the soft metal does not comprise a natural mixture of isotopes.

Embodiment 66. The method of embodiment 64 or 65, wherein incubating the element tagged affinity reagent with the analyte comprises:
incubating two or more differential element tagged affinity reagents with two or more analytes, wherein the element tagged affinity reagents specifically bind with the two or more analytes to produce two or more differentially tagged analytes, wherein analyzing the element tag bound to the affinity reagent comprises analyzing the differential element tags bound to the two or more analytes by mass spectrometric atomic spectroscopy.

Embodiment 67. The method of any one of embodiments 64 to 66, wherein the affinity reagent is further labeled with a fluorescent label.

Embodiment 68. The method of any one of embodiments 64 to 67, wherein the mass spectrometric atomic spectroscopy is ICP-MS.

Embodiment 69. The method of any one of embodiments 64 to 67, wherein the mass spectrometric atomic spectroscopy is by a mass spectrometer based flow cytometer.

Embodiment 70. The method of any one of embodiments 64 to 69, wherein the affinity reagent is an antibody.

Embodiment 71. The method of any one of embodiments 64 to 70, wherein the affinity reagent specifically binds biotin.

Embodiment 72. The method of any one of embodiments 64 to 69, wherein the affinity reagent is an oligonucleotide.

Embodiment 73. The method of any one of embodiments 64 to 72, wherein the element tagged affinity reagent is configured to bind to an analyte in a biological sample, and the biological sample comprises cells.

Embodiment 74. The method of any one of embodiments 64 to 73, wherein the soft metal is selected from Re, Pt, Pd, Nb, Tc, Hg, Ag, Au, Mo, Ru, Rh, Cd, W, Os, or mixtures thereof.

Embodiment 75. The method of any one of embodiments 64 to 74, wherein the soft metal is an element that does not naturally occur in the biological sample.

Embodiment 76. The method of any one of embodiments 54 to 75, wherein the element tag is or comprises a compound of Formula I as defined in any one of embodiments 1 to 33, or a compound of Formula II as defined in embodiment 34.

The above disclosure generally describes the present disclosure. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

### Example 1

### Preparation of Chelating Polymer Compound 1-1

Polymers having dipicolylamine (DPA) chelators attached to pendant groups in which pendant groups can chelate rhenium that extend the number of mass channels useful for mass cytometry were synthesized. Synthesis of the metal-chelator polymers and the materials used are described.

The exemplary chelating polymer compound I-1 of the present disclosure was prepared according to Scheme 2. An exemplary activated ester polymer 2-1 was reacted with an exemplary Lys-DPA chelator 1-4. The resulting polymer 2-2 was then attached to modifying groups including PEG and maleimide to obtain the compound I-1. The chelator 1-4 was synthesized according to Scheme 1. It can be appreciated that other compounds of the present disclosure can be made using similar methods, techniques and principles as described below with suitable modifications.

### Materials

Triethylamine (TEA, cat. no. 471283), acryloyl chloride (cat. no. 549797), 2-(dodecylthiocarbonothioylthio)-2-methylpropanoic acid (DDMAT), cat. no. 723010), 2,2′-azobis(2-methylpropionitrile) (AIBN, cat. no. 44109), Nε-Boc-L-lysine (cat. no. 359661), sodium triacetoxyborohydride (STAB, cat. no. 316393), 2-pyridinecarboxaldehyde (cat. no. P62003), HCl (4M in dioxane, cat. no. 345547), Tris(2-carboxyethyl)phosphine hydrochloride solution (TCEP, cat. no. 646547) were obtained from Sigma Aldrich. Pentafluorophenol was purchased from Matrix Scientific (cat. no. 006058). mPEG₆-NH₂ (cat. no. 281204) was obtained from ChemPep. Bis-Mal-PEG₆ (cat. no. BP-22152) was obtained from BroadPharm. 4-(4,6-dimethoxy-1,3,5-triazin-2-yl)-4-methylmorpholinium chloride (DMTMM, cat. no. D461245) was purchased from Toronto Research Chemicals. All organic solvents (anhydrous) were obtained from commercial sources and used without further purifications.

### Synthesis of Lysine-Based Rhenium Chelator

Chelator 1-4 was prepared in three steps, as shown in Scheme 1, by (i) direct reductive alkylation of a Boc protected lysine precursor,²⁰ (ii) Boc deprotection by hydrochloric acid (HCl in dioxane), followed by (iii) conversion of the amine hydrochloride salt to free base with NaOH. Reaction intermediates and the resulting product were characterized by ¹H-NMR to confirm their structures (FIG. 6).

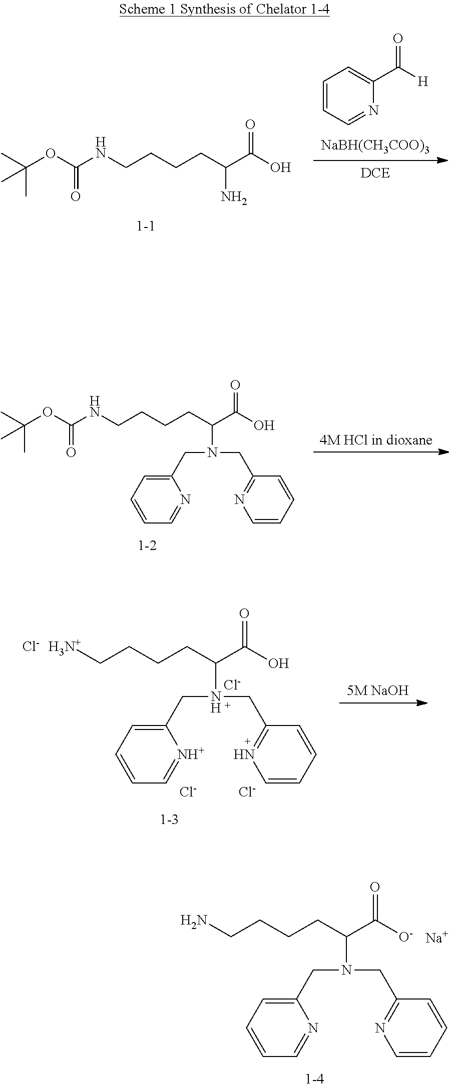

To a mixture of Nε-Boc-L-lysine 1-1 (2 g) and STAB (4.5 g) in dichloroethane (45 mL) was added 2-pyridinecarboxaldehyde (1.85 g, dissolved in 5 mL dichloroethane) at 0° C. under nitrogen. The suspension was stirred at room temperature for ca. 5 h and became homogeneous bright yellow. The reaction mixture was decomposed with deionized water (30 mL) and diluted with dichloroethane (100 mL). The separated organic layer was washed with deionized water (30 mL×4), dried with Na₂SO₄, and concentrated by rotary evaporation to obtain the tert-butyloxycarbonyl (Boc) protected lysine-based chelator 1-2 (2.5 g). ¹H NMR (600 MHz, CDCl₃) δ 8.53 (ddd, J=5.1, 1.8, 0.9 Hz, 2H), 7.65 (td, J=7.7, 1.8 Hz, 2H), 7.32 (dt, J=7.9, 1.1 Hz, 2H), 7.20 (ddd, J=7.6, 5.0, 1.2 Hz, 2H), 4.70 (s, 1H), 4.10 (d, J=3.8 Hz, 4H), 3.47 (dd, J=7.8, 6.5 Hz, 1H), 3.16-3.07 (m, 2H), 2.04-1.92 (m, 1H), 1.88-1.72 (m, 1H), 1.52-1.45 (m, 4H), 1.43 (s, 9H).

To remove the Boc group, a hydrogen chloride solution (4M in dioxane, 7 mL) was slowly added to a solution of the above compound I-2 (1.5 g) in DCM (5 mL) at 0° C. The resulting solution was stirred for 24 h at room temperature. A yellow precipitate was formed during the reaction. The solvent was discarded, and the precipitate was dissolved in methanol (5 mL) and precipitated into diethyl ether (15 mL). The precipitate was collected by centrifugation (2700×g, 10 min). This dissolution-precipitation cycle was repeated once more. Finally, the precipitate was dried in a vacuum oven at room temperate for 24 h to obtain the Boc deprotected lysine-based DPA chelator as an amine salt 1-3 (1.3 g). ¹H NMR (600 MHz, D₂O) δ 8.68 (ddd, J=6.0, 1.6, 0.7 Hz, 2H), 8.49 (td, J=7.9, 1.6 Hz, 2H), 8.05 (dt, J=8.0, 1.0 Hz, 2H), 7.92 (ddd, J=7.5, 5.9, 1.3 Hz, 2H), 4.42 (q, J=16.5 Hz, 4H), 3.57-3.51 (m, 1H), 2.96 (t, J=7.7 Hz, 2H), 1.92 (ddd, J=9.9, 8.4, 5.8 Hz, 1H), 1.89-1.82 (m, 1H), 1.69-1.61 (m, 2H), 1.52-1.44 (m, 2H).

To convert the amine salt to the free base, concentrated NaOH solution (5M) was slowly added to a solution of the amine salt (0.5 g dissolved in 2 mL of water) at 0° C. until a pH of ˜13 was reached. The basified solution was then lyophilized to obtain a light brown solid. Dichloromethane (5 mL) was added to dissolve the free base. The undissolved solids were removed by centrifugation (16000×g, 10 min). The supernatant was collected, concentrated, and dried under vacuum at room temperature for 24 h to obtain the final lysine-based DPA chelator as a free base 1-4 (0.3 g). ¹H NMR (500 MHz, D₂O) δ 8.24 (ddd, J=5.0, 1.8, 0.9 Hz, 2H), 7.60 (td, J=7.7, 1.8 Hz, 2H), 7.37 (dt, J=7.9, 1.2 Hz, 2H), 7.14 (ddd, J=7.6, 5.0, 1.2 Hz, 2H), 3.95 (d, J=14.6 Hz, 2H), 3.79 (d, J=14.6 Hz, 2H), 3.21 (dd, J=8.5, 6.3 Hz, 1H), 2.59 (t, J=6.8 Hz, 2H), 1.75-1.64 (m, 2H), 1.45-1.26 (m, 4H).

### Synthesis of Pentafluorophenyl Acrylate (PFPA) Monomer

In order to prepare activated ester polymer 2-1, the pentafluorophenyl acrylate (PFPA) monomer was first synthesized by slowly adding triethylamine (18.3 mL) to a solution of pentafluorophenol (20 g in 130 mL of dichloromethane) at 0° C. under nitrogen followed by 10.6 mL of acryloyl chloride. The reaction mixture was stirred at 0° C. for 2 h and then at room temperature overnight. The salt was removed by filtration. The solution was concentrated by rotary evaporation and then purified using a silica gel column chromatography with hexane as the eluent. ¹H NMR (500 MHz, CDCl₃) δ 6.71 (dd, J=17.3, 1.0 Hz, 1H), 6.37 (dd, J=17.3, 10.6 Hz, 1H), 6.17 (dd, J=10.5, 0.9 Hz, 1H). ¹⁹F NMR (564 MHz, CDCl₃) δ −153.17-−153.27 (m), −158.74 (t, J=21.5 Hz), −163.11 (td, J=23.0, 22.5, 5.4 Hz).

### RAFT Polymerization of PFPA Monomer

Synthesis of activated ester polymer 2-1, poly(pentafluorophenyl acrylate) (PPFPA) was achieved by reversible addition-fragmentation chain transfer (RAFT) polymerization.^{21,22} The polymerization was carried out in a Schlenk flask equipped with a stir bar at 70° C. using 2-(dodecylthiocarbonothioylthio)-2-methylpropanoic acid (DDMAT, 0.28 mmol) as the chain transfer agent (CTA) and 2,2′-azobis(2-methylpropionitrile) (AIBN, 0.028 mmol) as the thermal initiator in 1,4-dioxane (6.0 mL). The solution was degassed by three freeze-pump-thaw cycles after which the flask was sealed and put into a preheated oil bath (70° C.) for 9 hours. After polymerization, the solution was cooled to room temperature by cold water and exposed to air. The polymer was precipitated into excess cold hexane (30 mL). The polymer obtained was dissolved in chloroform (5 mL) and precipitated again into hexane (30 mL). This dissolution-precipitation process was repeated for 3 times. The final polymer, poly(PFPA) 2-1 was obtained as yellow powder after drying in a vacuum at room temperature overnight.

A monomer-to-CTA to initiator molar ratio ([M]:[CTA]:[I]) of 30:1:0.1 was chosen to tailor the molecular weight of the resulting polymer 2-1, with an apparent number average molecular weight (Mₙ^{GPC}) of 9,400 g/mol and a relatively narrow molecular weight distribution (D=1.25) being obtained at ca. 73% conversion (FIG. 4).

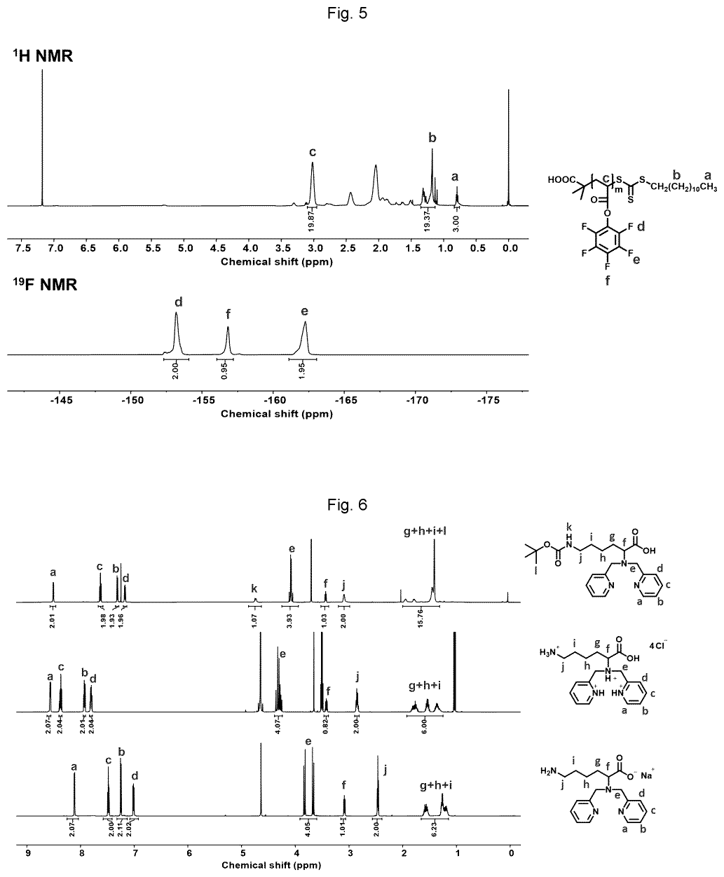

The degree of polymerization (DP) was determined using ¹H NMR spectroscopy by comparing the integration of the PPFPA backbone peaks (δ=3.11 ppm) with that of the methyl unit for the dodecyl chain end (δ=0.88 ppm) (FIG. 5 S2). The DP of the corresponding polymer 2-1 was ca. 20. An ¹⁹F NMR spectrum of polymer 2-1 displayed three broad peaks at −153.2, −156.8, and −162.3 ppm with an integration ratio of 2:1:2 corresponding to the pentafluorophenyl groups along the polymer backbone (FIG. 5).

Aminolysis of Poly(PFPA) with Lysine-Based Rhenium Chelator

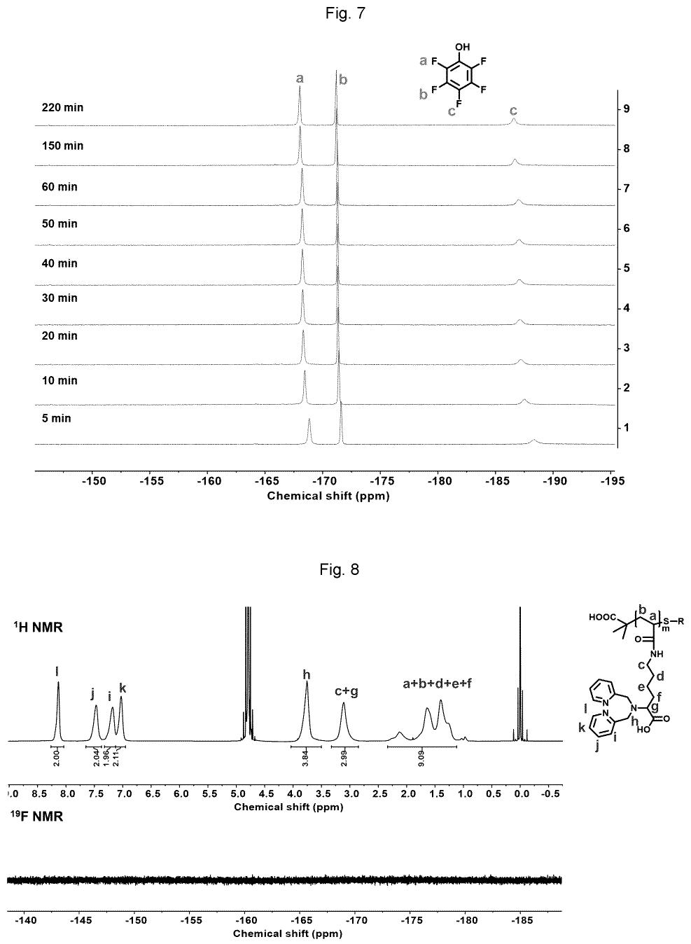

Next, polymer 2-1 was treated with a slight excess of a lysine-based chelator 1-4, (1.6 equivalents with respect to PFPA repeating units) at room temperature to obtain polymer 2-2 (Scheme 2, step a). Chelator 1-4 was prepared in three steps, as shown in Scheme 1 above. The aminolysis reaction was monitored by ¹⁹F NMR spectroscopy. Overtime, broad signals corresponding to PFPA units along the backbone disappeared, while sharp signals corresponding to the released pentafluorophenol appeared (FIG. 7). After stirring overnight at room temperature (13 h), 100 μL of ethanolamine was added to the reaction mixture and the reaction mixture was stirred for 3 h. The polymer was precipitated into diethyl ether (50 mL) and then dissolved in H₂P. Excess chelators were removed using a spin filter (Amicon, Ultra-15, 3 kDa), washed twice with H₂O, thrice with PBS buffer and thrice with H₂O. Polymer 2-2 (polyDPA) was obtained as light brown powder after freeze drying.

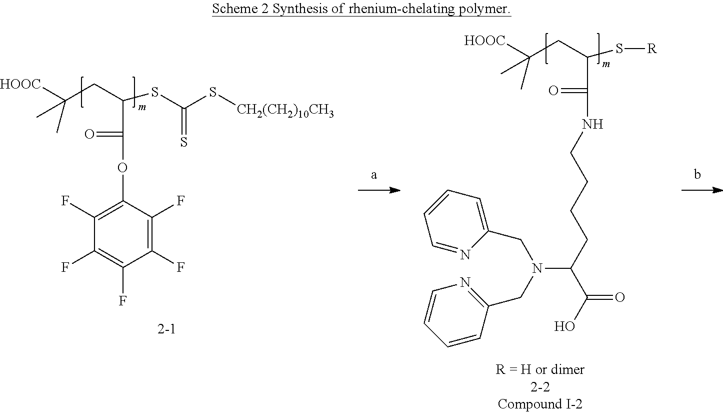

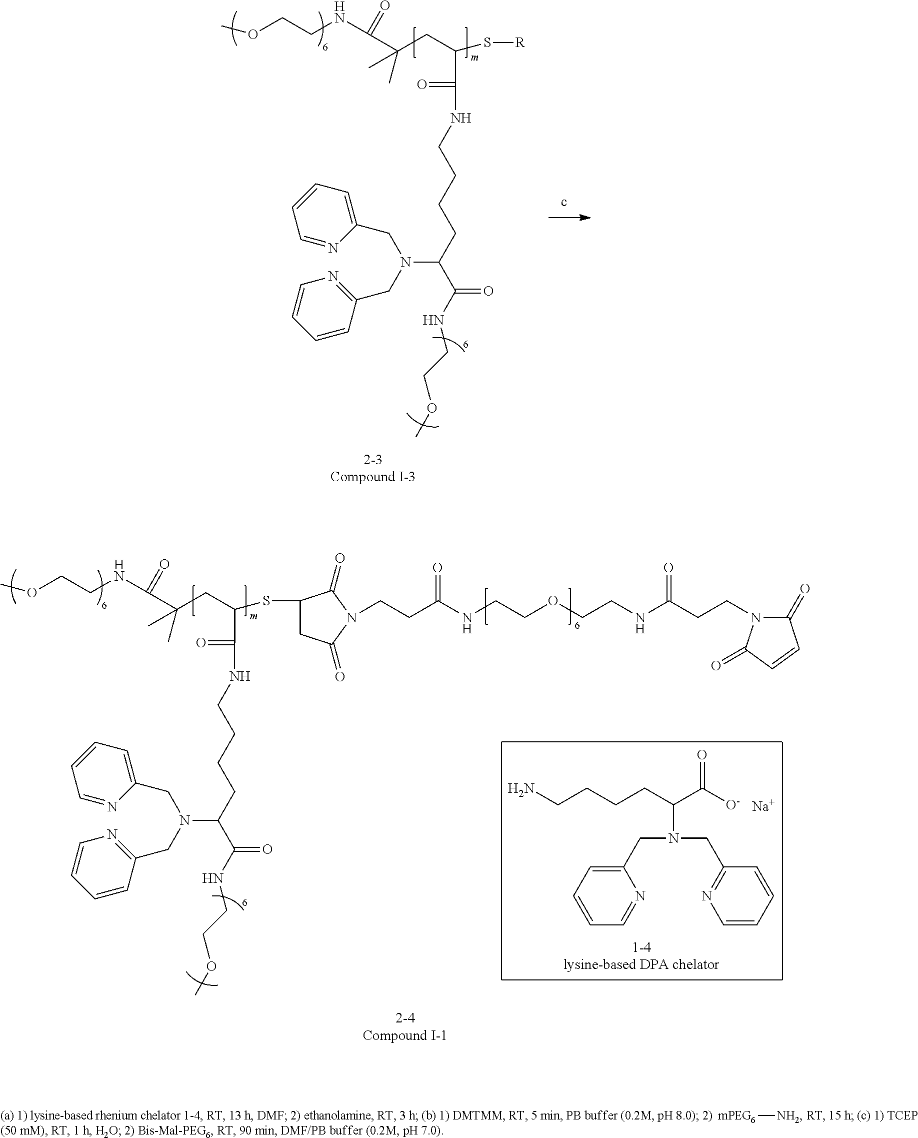

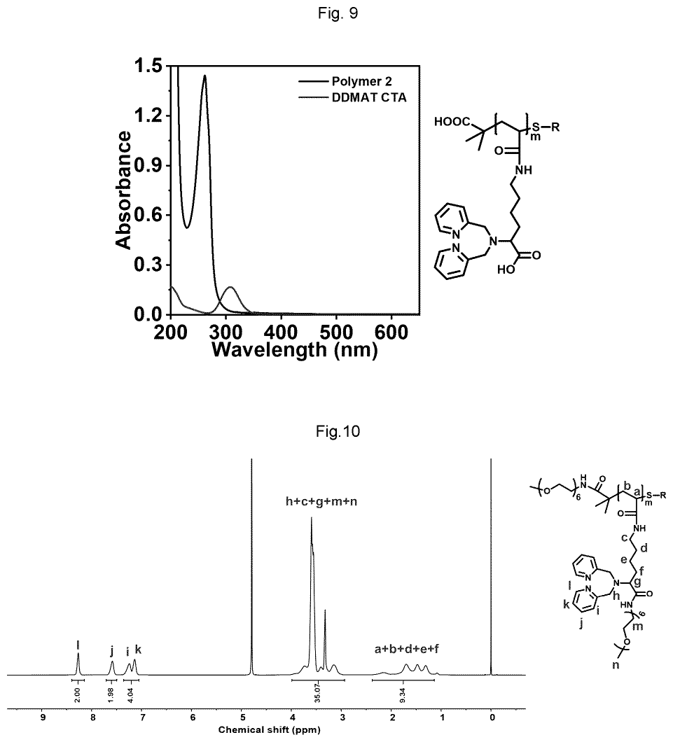

After purification, the ¹H NMR of polymer 2-2 showed a set of resonances corresponding to the introduction of chelator 1-4 while the ¹⁹F spectrum displayed no signal (FIG. 8). These NMR studies suggest complete modification of the PFPA units with near-quantitative incorporation of chelator 1-4 along the polymer backbone. In addition, the UV-vis spectrum of polymer 2-2 in methanol showed negligible absorbance at 309 nm, suggesting the cleavage of the trithiocarbonate group during aminolysis (FIG. 9). A sharp absorbance peak located at 262 nm, was however observed corresponding to the absorption from the 2-pyridyl groups of the chelator.

The carboxylic acid (COOH) moiety of 1-4 can serve two purposes. First, it is anticipated that the carboxylate ion would improve the water solubility of the metal-loaded polymer. Second, this metal-polymer complex can have a net positive charge. Polymers with a positive charge in each pendant group can interact non-specifically with cells that commonly have a negatively charged outer membrane. The carboxylate provides a potential counterion, so that each pendant group is zwitterionic. It was found that rhenium-loaded polymer 2-2 had low solubility in water or in PBS buffer. To further improve aqueous solubility, polymer 2-2 was modified with a short methoxy polyethylene glycol (mPEG₆-NH₂) as shown in Scheme 2, step b. The goal here was not only to enhance the water solubility of the polymer, but to provide a PEG corona to shield the positively charged complex from interaction with cells.

PEGylation of Polymer 2-2 with mPEG₆-NH₂

Polymer 2-2 (50.3 mg) was first treated with excess (4-(4,6-dimethoxy-1,3,5-triazin-2-yl)-4-methyl-morpholinium chloride) (DMTMM, 288 mg in 1 mL H₂O, ca. 8 molar equivalents to each carboxylic group) in PB buffer (4 mL, 0.2M, pH 8.0) (Scheme 2, step b). The reaction mixture was stirred at room temperature for 5 min to activate the carboxylic acid functional groups. Following this, excess mPEG₆-NH₂ (ca. 7 molar equivalents to each carboxylic group) was quickly added and the reaction solution was stirred overnight (15 h) at room temperature. The PEGylated version of polymer 2-2 was subsequently obtained, herein referred to as polymer 2-3. Polymer 2-3 was purified using a spin filter (Amicon, Ultra-15, 10 kDa), washed thrice with H₂O, twice with PBS buffer and thrice with H₂O. The final polymer, polyDPA-mPEG₆ 2-3, was obtained as light brown solid after freeze drying.

The ¹H NMR spectrum of polymer 2-3 confirms PEGylation, with the appearance of new peaks at 3.32 ppm and 3.59 ppm corresponding to the methoxy group and the ethylene glycol repeating units of the mPEG₆(FIG. 10). A rhenium loading experiment confirmed that metal-loaded polymer 2-3 was soluble in both water and PBS buffer.

### Attaching Maleimide Functional Group to Polymer 2-3

Polymer 2-3 was further modified to install maleimide functional groups (Scheme 2, step c). First, any disulfide bonds that may have formed in prior steps were reduced with tris(2-carboxyethyl)phosphine (TCEP). To a solution of polymer 2-3 (10 mg) in H₂O (1 mL), TCEP (80 μL, 0.5 M) was added. The concentration of TCEP in the reaction solution was ca. 50 mM. The reaction mixture was stirred at room temperature for 1 h. Then, the polymer was washed twice with acetic acid solution (ca. 5 mM, pH 3.5) using a spin filter (Amicon, Ultra-15, 10 kDa) to remove excess TCEP.

The newly reduced thiol groups were then reacted with an excess of a bismaleimide (Bis-Mal-PEG₆) to afford the maleimide-functionalized compound I-1. Immediately afterwards, the concentrated polymer solution (ca. 300 μL) was transferred to a 2-dram glass vial, 0.5 mL of PB buffer (0.2 M, pH 7.0) was added followed by Bis-Mal-PEG₆ (22 mg in 120 μL DMF). The reaction solution was stirred at room temperature for 90 min. The compound I-1 was then purified using a spin filter (Amicon, Ultra-15, 10 kDa), washed twice with H₂O, once with PB buffer (0.2 M, pH 7.0) and thrice with H₂O. After washing, the concentrated polymer solution was centrifuged at 12000×g for 10 min to remove undissolved solids. The supernatant was taken and lyophilized to obtain the final polymer, polyDPA-mPEG₆-Mal compound I-1 as light brown solid.

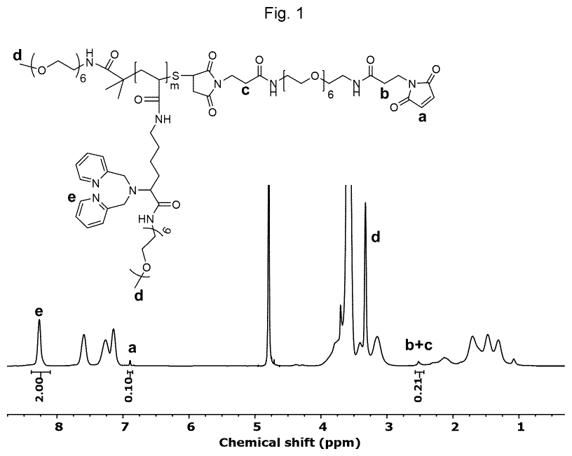

The ¹H NMR spectrum of compound I-1 showed the appearance of a new peak at 6.89 ppm corresponding to the maleimide (FIG. 1). The ratio of integration of this peak to that of the chelator protons at 8.27 ppm is 1:20, which suggests nearly quantitative incorporation of the maleimide functional group into the polymer.

Rhenium loading to compound I-1

Metal loading was achieved by incubating compound I-1 (2.1 mg in 2 mL anhydrous MeOH) in a 2-dram glass vial with a slight excess of rhenium salt [NEt₄]₂[Re(CO)₃Br₃]²⁴ (3 mg, 1.2-fold to each chelator, dissolved in 0.5 mL anhydrous MeOH. The reaction mixture was incubated at 37° C. for 2 h without stirring. After incubation, the solution was poured into an Amicon spin filter (Ultra-15, 10 kDa) prefilled with H₂O (12 mL). The polymer was washed thrice with H₂O. After washing, the polymer solution was lyophilized to obtain the rhenium-loaded polymer.

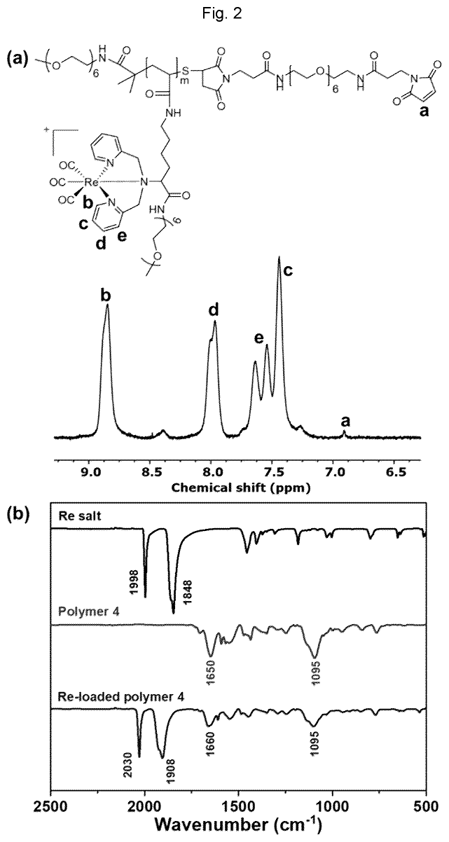

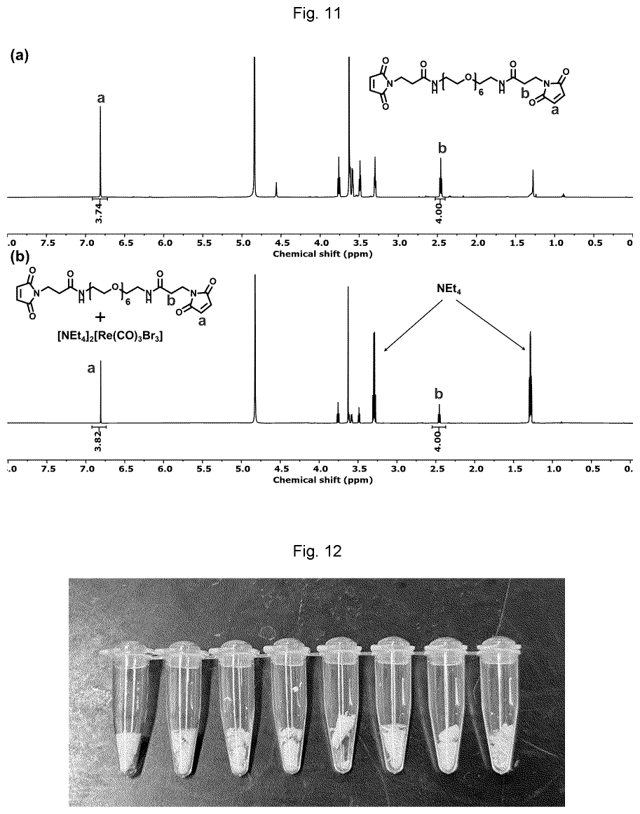

FIG. 2 (a) presents a portion of the ¹H NMR of the Re-loaded compound I-1 (compound II-1) in the aromatic region. Compared to the unloaded polymer, all pyridyl proton signals were shifted downfield due to the electron withdrawing inductive effects of Re(l). Importantly, the maleimide group survived under these reaction conditions (FIG. 11). Successful loading was further confirmed by Fourier transform infrared (FTIR) spectroscopy as shown in FIG. 2(b). The rhenium salt showed two strong carbonyl absorptions at 1848 and 1998 cm⁻¹. Compound 1-1 showed absorption bands at 1650 and 1095 cm⁻¹, corresponding to the C═O stretching of amide group and vibrations of ether C—O—C bond in PEG, respectively.²⁵ After loading, two new absorption bands corresponding to carbonyl groups appeared at 1908 and 2030 cm⁻¹, suggesting the presence of the fac-[Re(CO)₃]⁺ core. The rhenium-loaded polymer can be lyophilized for long-term storage and redissolved in buffers before bioconjugation (lyophilized sample shown in FIG. 12).

### Example 2

The Re-loaded polymer compound II-1 was labelled with a primary antibody and used in a mass cytometry immunoassay.

### Antibody Labelling of Re-Loaded Polymer

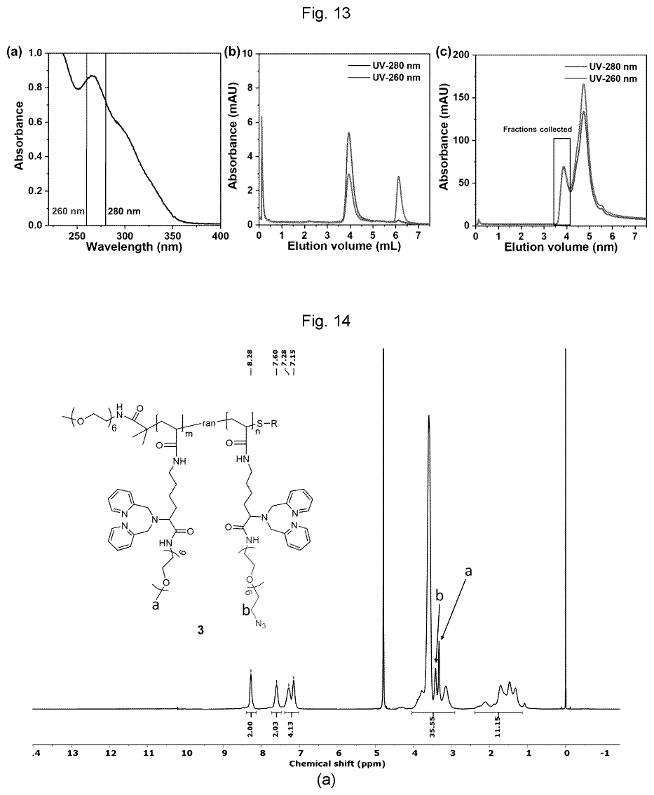

To evaluate the performance of the exemplary element tag compound II-1 in mass cytometry immunoassays, a primary antibody, CD20, was labeled with the polymer tag following standard Maxpar™ antibody labeling protocol. Briefly, the antibody was partially reduced by TCEP, washed in a spin filter, and then mixed with an excess of polymer, and the mixture was incubated at 37° C. for 1 h. The antibody-polymer conjugate was purified by fast-protein liquid chromatography to remove excess unconjugated polymers (FIG. 13).

### Antibody Titration Experiments

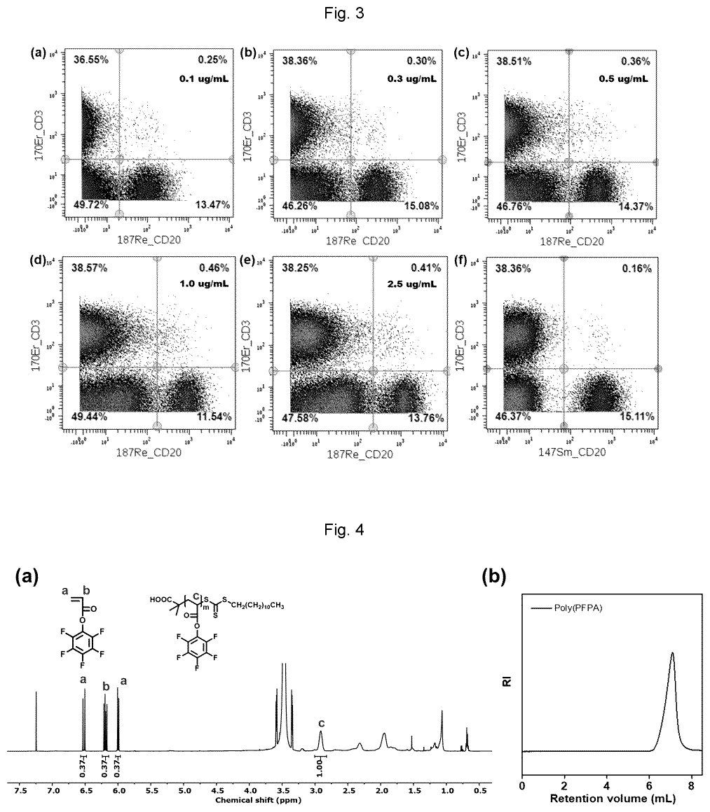

Antibody titration experiments were then performed with human peripheral blood mononuclear cells (PBMCs) to evaluate the performance of the purified conjugate. In these experiments, human PBMCs were stained with a 4-plex antibody panel (Fluidigm Maxpar™ reagents), including ¹⁵⁴Sm-CD45, ¹⁶⁰Gd-CD14, ¹⁷⁰Er-CD3 and ^{187/185}Re-CD20. A separate staining panel, where ^{187/185}Re-CD20 was replaced with ¹⁴⁷Sm-CD20, was used as a positive control. ^{187/185}Re-CD20 conjugates were titrated at concentrations of 0.1, 0.3, 0.5, 1 and 2.5 μg/mL. As shown in FIG. 3, ^{187/185}Re-CD20 allows distinct separation of CD20+B-cell subsets from the rest of cell subsets in PBMCs. In addition, highly comparable percentages of major cell subsets within PBMCs were achieved by using either ^{187/185}Re-CD20 at 0.3 μg/mL (FIG. 3(b)) or ¹⁴⁷Sm-CD20 at optimal titer (FIG. 3(f)). These results suggest that the rhenium tagged antibody provides accurate quantification for single-cell immunophenotyping experiments and can be used in conjugation with commercial reagents for mass cytometry immunoassays.

### Example 3

Chelators with One or More Platinum Atoms Chelated by DPA

Polymers having dipicolylamine (DPA) chelators attached to pendant groups in which pendant groups can chelate platinum useful for mass cytometry were synthesized. Synthesis of exemplary metal-chelator polymers with platinum and the materials used are described.

### Materials

Polymer 2-2 as in Example 1 was used to prepare Polymer 3-2 (Scheme 3). Azido-PEG₆-NH₂ (cat. no. 76172) and potassium tetrachloroplatinate (K₂PtCl₄, cat. no. 520853) were obtained from Sigma Aldrich.

PEGylation of polymer 2 with azide-PEG₆-NH₂

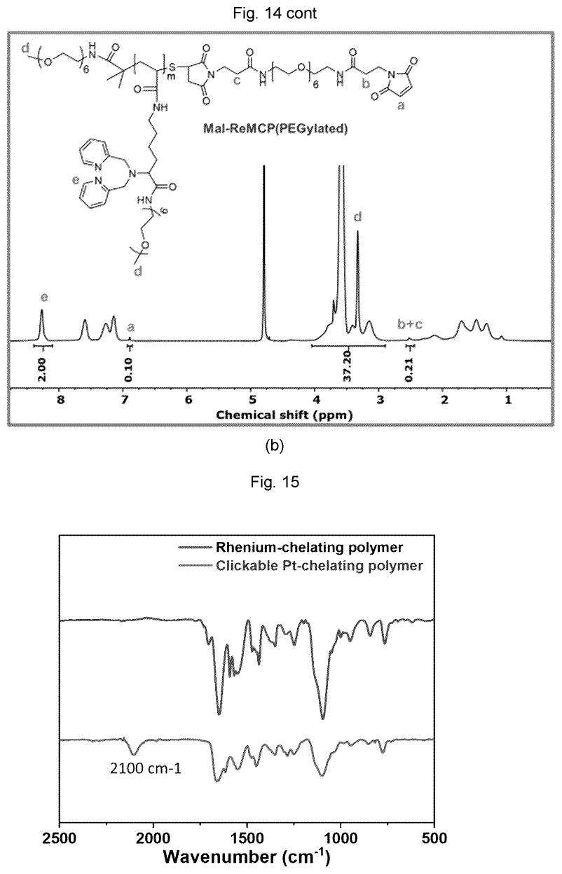

1H-NMR was employed to characterize the resulting polymer 3-2 (FIG. 14). FTIR further confirms successful incorporation of azide groups (FIG. 15). To a solution of end-capped polymer 3-1 (20 mg in 2 mL PB buffer, 0.2M, pH 8.0), DMTMM solution (124 mg in 0.5 mL H₂O, ca. 8-fold to each COOH) was added. The solution was stirred at room temperature for 5 min to activate the COOH groups. After 5 min, a mixture of PEGs containing mPEG₆-NH₂ (54 mg, 3.5-fold equivalents to each COOH) and azide-PEG₆-NH₂ (64 mg, 3.5-fold equivalents to each COOH) was added. The reaction mixture was stirred at room temperature overnight. The resulting polymer 3-2 was purified using a spin filter (Amicon, Ultra-5, 10 kDa), washed three times with H₂O, twice with PB buffer (0.2 M, pH 7.6) and three times with H₂O. The polymer solution was then lyophilized overnight to obtain the final product 3-2.

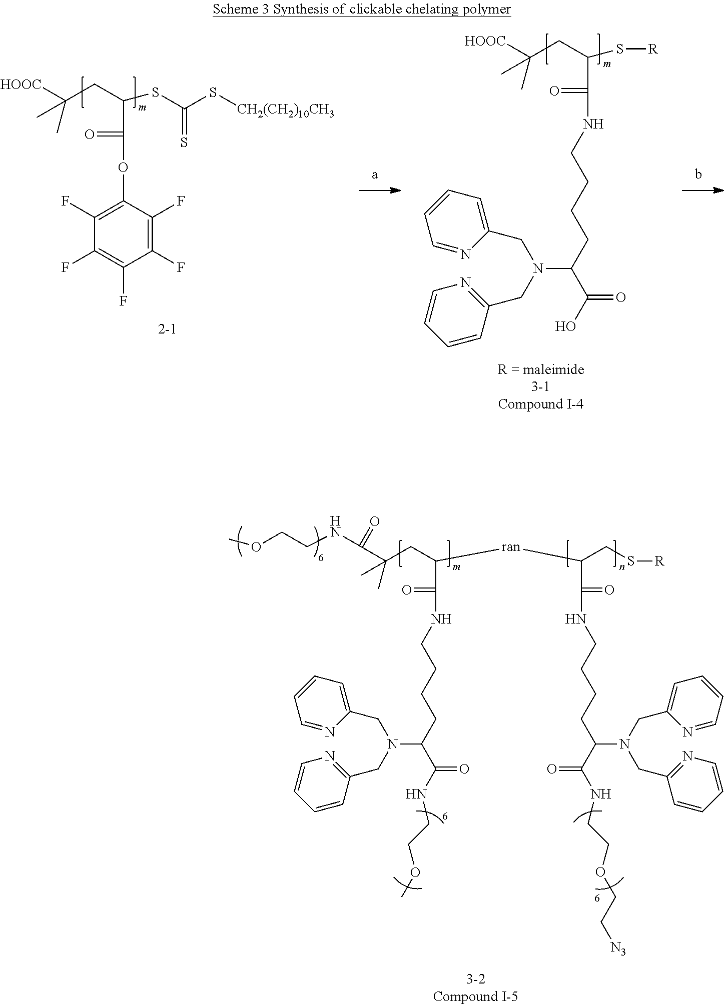

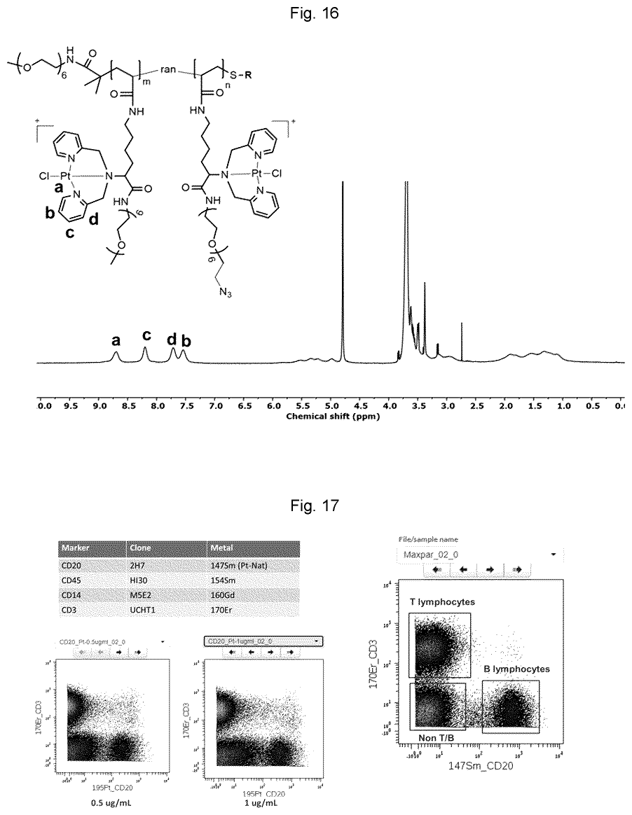

Platinum loading to polymer 3-2 Platinum loading on to polymer 3-2 was carried out according to Scheme 4). ¹H-NMR was employed to confirm successful chelation of Pt (FIG. 16). To a solution of polymer 3-2 (5.8 mg in 5.5 mL anhydrous MeOH) in a 2-dram (˜7 mL) glass vial, a solution of K₂PtCl₄ (200 μL, 50 mM in DMSO, 1.2-fold equivalents to each chelator) was added. The vial was wrapped with aluminum foil to avoid exposure to daylight. The reaction mixture was incubated at 45° C. in an oil bath for 2 h without stirring. After incubation, the solution was poured into an Amicon spin filter (Ultra-15, 10 kDa) prefilled with H₂O (10 mL). The resulting solution of polymer 4-1 (Compound 11-2) was washed thrice with NaCl solution (20 mM) and once with H₂O. After washing, the polymer solution was lyophilized to obtain the Pt-loaded polymer 4-1.

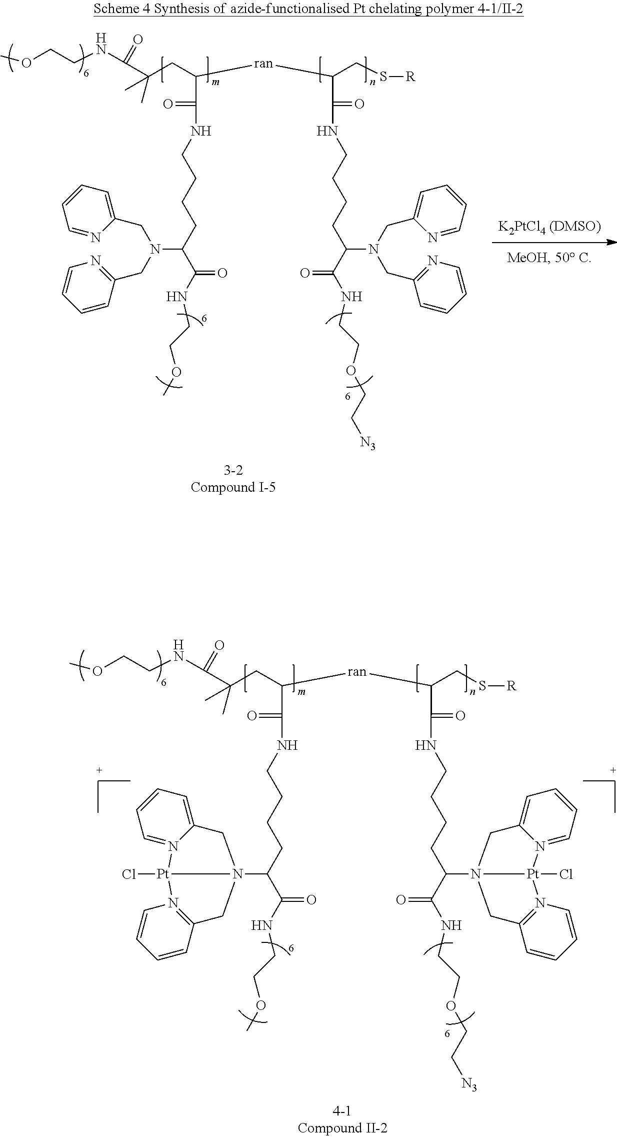

### Example 4

The Pt-loaded polymer was labelled with a primary antibody and used in a mass cytometry immunoassay.

### Antibody Labelling of Pt-Loaded Polymer

To evaluate the performance of the polymer element tag 4-1/II-2 in mass cytometry immunoassays, a primary antibody, CD20, was labeled with the polymer element tag 4-1/II-2. To a solution of dibenzocyclooctyne (DBCO) modified CD20 Ab (160 μg, 1.6 mg/mL), a solution of Pt-loaded polymer 4-1 was added (0.27 mg in 50 μL H₂O, ca. 13-fold molar excess to Ab). The reaction mixture was vortexed at room temperature for 2 h and then at 4° C. overnight. The conjugate was purified by washing five times with PBS using a spin filter (Amicon, Ultra-0.5, 100 kDa).

### Antibody Titration Experiments

Antibody titration experiments were performed as described below. FIG. 17 shows CD20-PolyPt was effective in separating B cells from T cells at a titer of either 0.5 or 1.0 μg/mL.

The antibody staining cocktails (70 μL) were prepared by mixing different Maxpar® MCP-Ab conjugates with the ^{nat}Pt-Ab conjugate. For immunoassays with the ^{nat}Pt-CD20, four antibody staining cocktails were prepared. One cocktail contained only Maxpar® MCP-Ab conjugate (i.e., ¹⁵⁴Sm-CD45, ¹⁶⁰Gd-CD14, ¹⁷⁰Er-CD3 and ¹⁴⁷Sm-CD20) and was used as the positive control. The other three cocktails consisted of both Maxpar® MCP-Ab conjugates (i.e., ¹⁵⁴Sm-CD45, ¹⁶⁰Gd-CD14 and ¹⁷⁰Er-CD3) and the ^{nat}Pt-CD20 conjugate, where the concentration of the ^{nat}Pt-CD20 conjugate in each cocktail was different for titers of 0.5, 1.0 and 2.5 μg/mL, respectively.

For the staining process, a PBMC suspension (ca. 3 million cells in 30 μL Maxpar® cell staining buffer, Fc blocked) was added to the antibody cocktail (70 μL). The mixture was gently vortexed and incubated at room temperature for 30 min. After incubation, cells were washed twice with cell staining buffer and then fixed with 1.6% formaldehyde/PBS solution at room temperature for 10 min. The fixed cells were pelleted and cell intercalation solution (Ir-intercalator, 1 mL, final concentration: 125 nM) was added. The cells were then incubated at 4° C. overnight. After incubation, cells were washed twice with cell staining buffer and twice with Maxpar® cell acquisition solution. The pelleted cells were resuspended in cell acquisition solution (1 million cells per mL) containing EQ™ Four Element Calibration Beads and subjected to mass cytometry analysis.

Uptake of CD20-PolyPt conjugates by monocytes was also observed.

### Example 5

Chelators with One or More Mercury Atoms Chelated by DPA

Polymers having dipicolylamine (DPA) chelators attached to pendant groups in which pendant groups can chelate mercury useful for mass cytometry were synthesized. Synthesis of exemplary mercury-chelator polymers and the materials used are described.

### Materials

PolyDPA polymer (2-3) was prepared as described in Example 1. Mercury acetate (cat. No. 176109) and methanol (cat. No. 322415) were purchased from Sigma Aldrich.

Hg loading to polyDPA (Polymer 2-3)

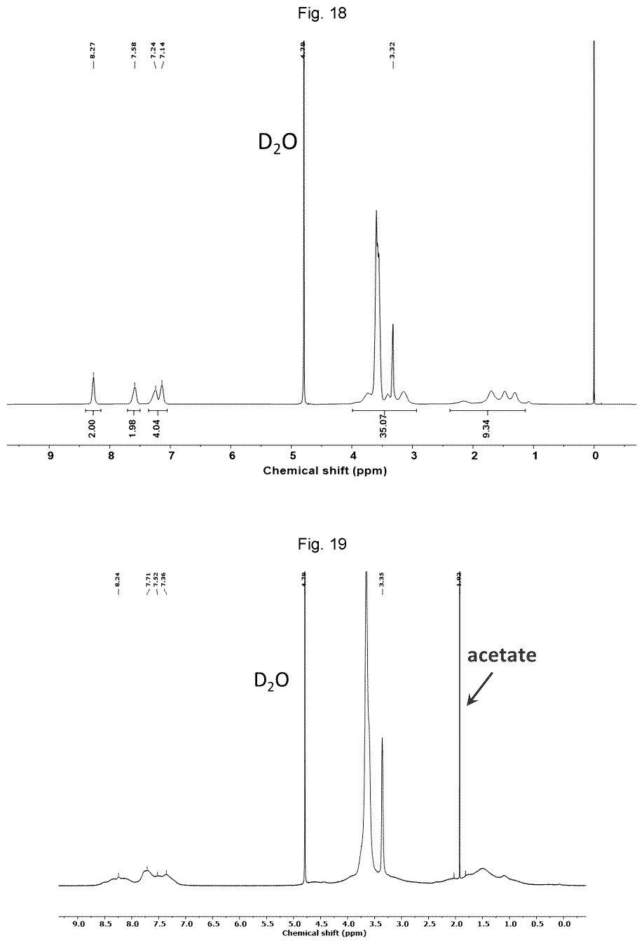

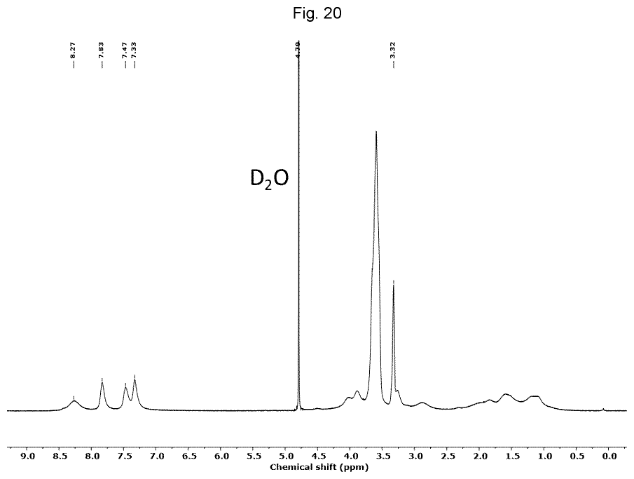

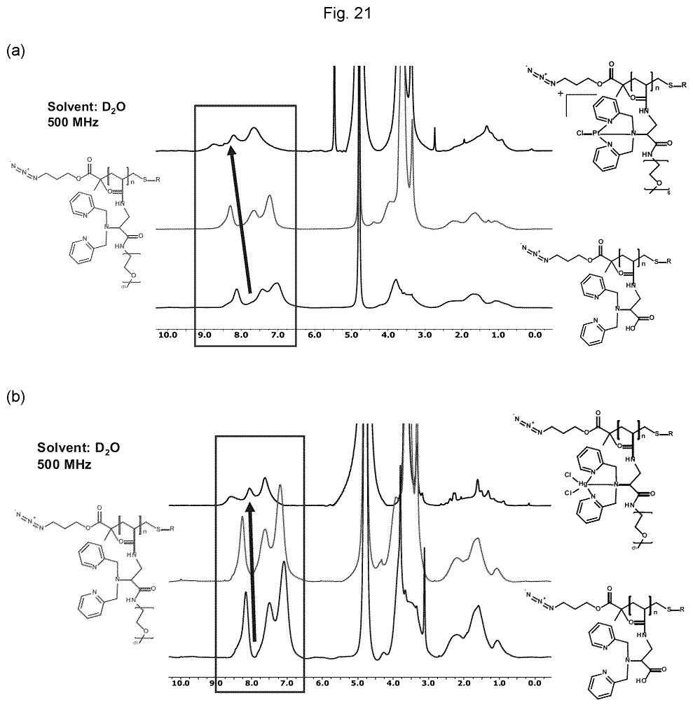

To a solution of polymer 2-3 (2.5 mg polymer in 2 mL methanol) in a 2 dram glass vial, a solution of Hg salt (1.5 mg Hg(OAc)₂ in 0.5 mL methanol, 1.4-fold excess to DPA chelators) was added followed by gentle swirling. The resulting solution was incubated at 40° C. for 2 h without stirring (Scheme 5). After incubation, the solution was poured to a spin filter (Amicon Ultra 4, 10 kDa) pre-filled with water (1.5 mL). The resulting solution of polymer 5-1/II-3 was washed three times with water (2700 g, 20 min) and then lyophilized overnight to obtain the final product. For the NMR measurement, the polymer 5-1/II-3 was redissolved in D₂0. ¹H-NMR was employed to confirm successful chelation of mercury (FIGS. 18 & 19)

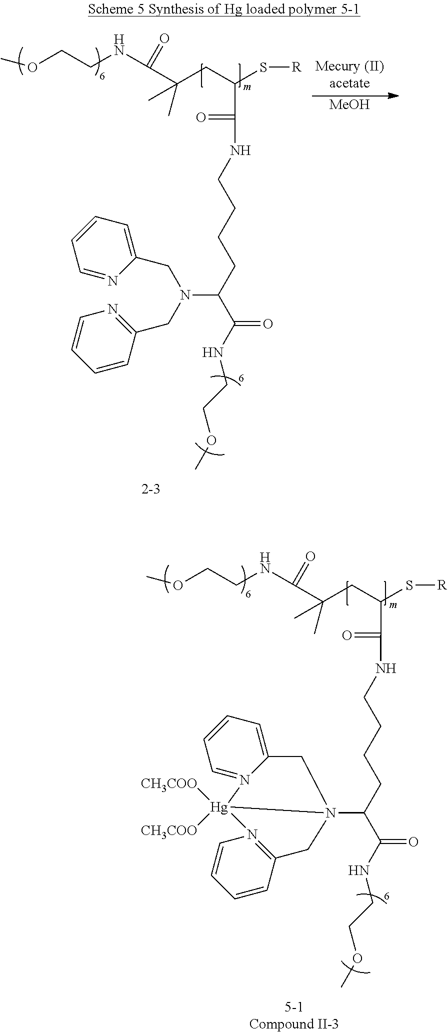

### Example 6

Chelators with One or More Silver Atoms Chelated by DPA

Polymers having dipicolylamine (DPA) chelators attached to pendant groups in which pendant groups can chelate silver useful for mass cytometry were synthesized. Synthesis of exemplary silver-chelator polymers and the materials used are described.

### Materials

Polymer 2-3 prepared as described in Example 1. Silver perchlorate (cat. No. 226548), and methanol (cat. No. 322415) were purchased from Sigma Aldrich.

Ag Loading to polyDPA (Polymer 2-3)

To a solution of polymer 2-3 (2.5 mg polymer in 2 mL methanol) in a 2 Dram glass vial, a solution of Ag salt (1.0 mg AgClO₄ in 0.5 mL methanol, 1.2-fold excess to DPA chelators) was added followed by gentle swirling. The resulting solution was incubated at 40° C. for 2 h without stirring (Scheme 6). After incubation, the solution was poured to a spin filter (Amicon Ultra 4, 10 kDa) pre-filled with water (1.5 mL). The resulting solution of polymer 6-1/II-4 was washed three times with water (2700 g, 20 min) and then lyophilized overnight to obtain the final product. For the NMR measurement, the polymer 6-1/II-4 was redissolved in D₂O. ¹H-NMR was employed to confirm successful chelation of silver (FIGS. 18 & 20)

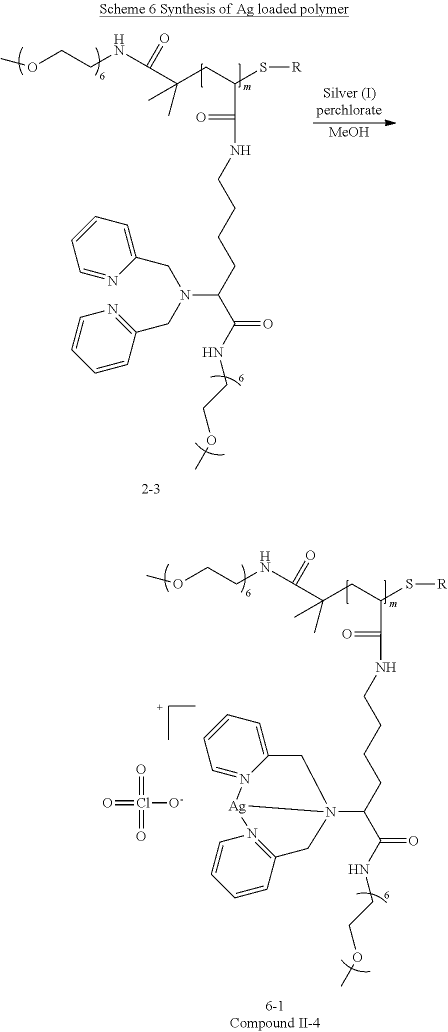

### Instrumentation

¹H NMR and ¹⁹F NMR experiments were performed on an Agilent DD2 500 MHz spectrometer or Agilent DD2 600 MHz spectrometer.

UV-vis measurements were performed on an Agilent Cary 300 UV-vis spectrophotometer.

FT-IR measurements were performed on a PerkinElmer Spectrum Two™ infrared spectrometer with an ATR accessory. All spectra were collected in the range of 500-4000 cm⁻¹ at a resolution of 1 cm⁻¹.

GPC measurements were performed on a Waters 515 HPLC equipped with a Viscotek VE 3580 refractive index (RI) detector. Tetrahydrofuran (THF) containing 2.5 g/L tetra-n-butylammonium bromide (TBAB) was used as the eluent (35° C., flow rate=0.6 mL/min). The system was calibrated with PMMA standards.

FPLC experiments were performed on the AKTA pure 25 L system. For the purification of the polymer-antibody conjugates, a Superdex™ 200 10/300 GL column was used.

Mass cytometry experiments were performed on a CyTOF® Helios™ system at Fluidigm Canada (Markham, ON). Data were obtained in FCS3.0 file format and processed by FlowJo software.

### Example 7

Preparation of Zwitterionic Poly(sulfobetaine methacrylate) PSBMA as Solubility Modifier

### Materials

4-Cyano-4-(phenylcarbonothioylthio)pentanoic acid (CTA agent), 2-(N-3-Sulfopropyl-N,N-dimethyl ammonium)ethyl methacrylate (SBMA), 4,4′-Azobis(4-cyanovaleric acid) (ACVA), 2,2,2-trifluoroethanol (TFE) were obtained from Sigma Aldrich.

### Experimental Details

In a 10 mL Schlenk flask, CTA (14.51 mg, 5.19×10⁻² mmol, 1.0 equiv.), SBMA (501 mg, 1.78 mmol, 35 equiv.), and ACVA (1.692 mg, 5.13×10⁻³ mmol, 0.1 equiv.) were dissolved in TFE (2.63 mL). The polymerization solution was subjected to 3 freeze-pump-thaw cycles. The polymerization solution was heated in pre-heated oil bath at 70° C. for 8 h. The polymerization was quenched by freezing the solution via plunging the Schlenk flask into liquid nitrogen. After thawing the polymerization solution, it was stored at 4° C. overnight. ¹H-NMR analysis of an aliquot of the crude polymerization solution indicated that the monomer conversion was ˜70%. Excess solvent was evaporated in vacuo using a rotary evaporator. The crude polymer mixture was re-dissolved in TFE (˜1 mL), precipitated in methanol (˜13 mL), and centrifuged at 2700 rcf for 5 min: this process was repeated 3 times. The resulting polymer was then dried in vacuo overnight to obtain the desired polymer. The solid product had distinctly segregated pink and white solids.

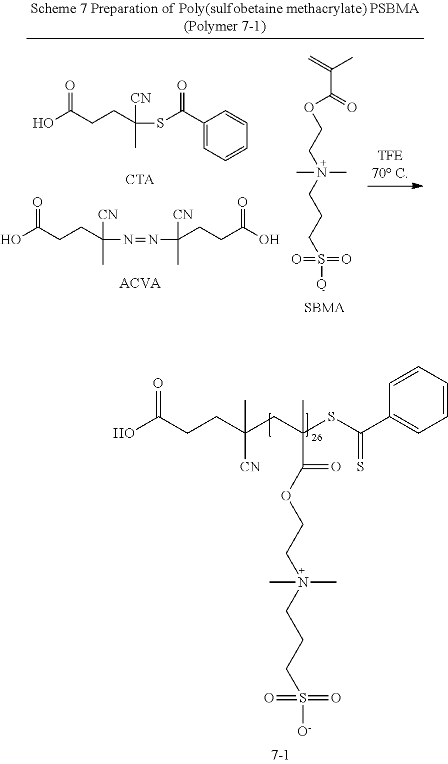

PSBMA such as polymer 7-1 can be attached to polymeric chelators such as compound 2-2, optionally through a diamine linker as shown in for example Scheme 8. For instance, 4-(4,6-dimethoxy-1,3,5-triazin-2-yl)-4-methyl-morpholinium chloride (DMTMM) or other similar chemicals can be used as a coupling agent.

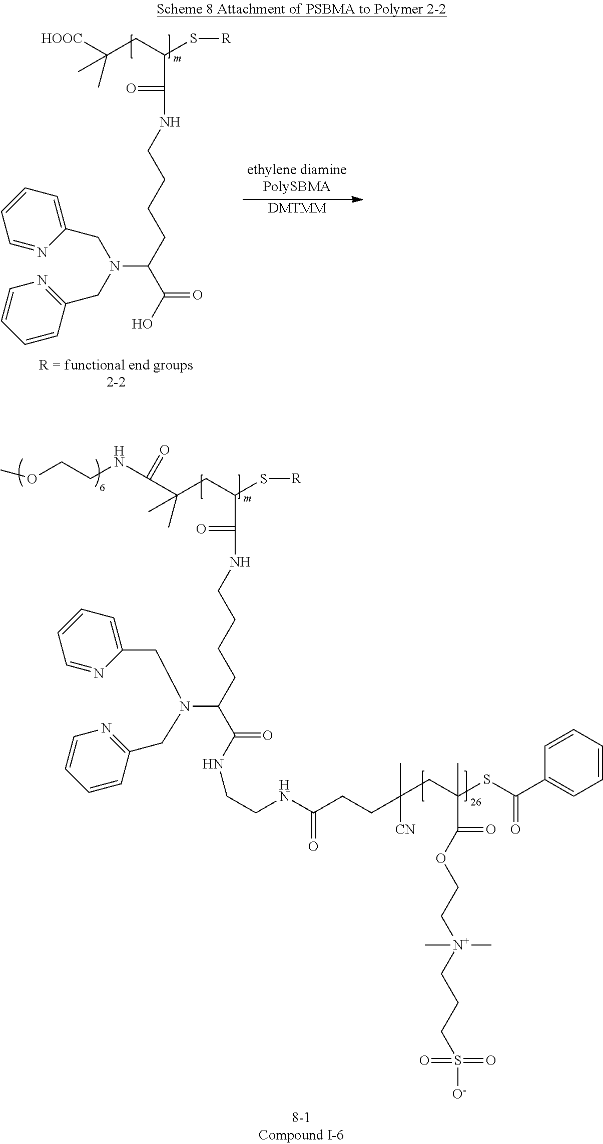

### Example 8 Preparation of Imidazole-Based Chelating Polymer

It can be appreciated that imidazole-based chelators can be prepared using methods similar to that used in Example 1 in the preparation of DPA-based chelators. An exemplary synthesis is shown in Scheme 9. Other examples of lysine-imidazole chelators include those described in Maresca et al., Bioconjugate Chem., 2010, 21, 1032-1042, the content of which is incorporated herein by reference in its entirety.

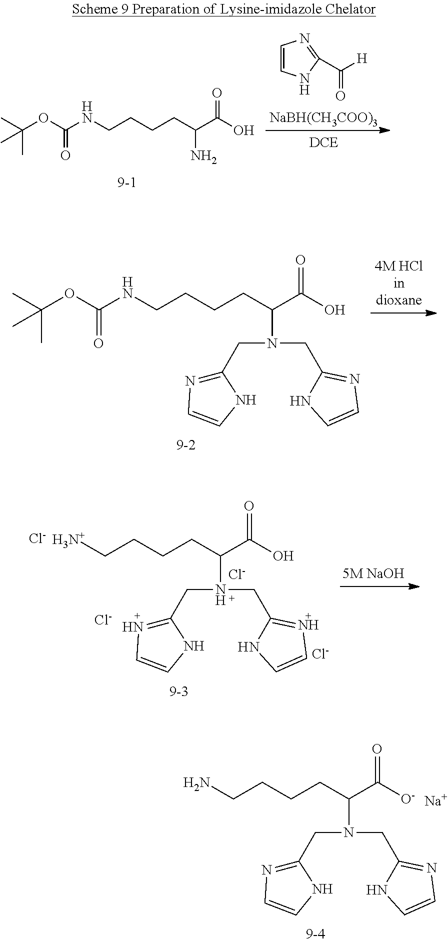

Nε-protected lysine 9-1 can be functionalised at the α-amino group with two imidazole groups by reductive amination and deprotected to arrive at the lysine-imidazole chelator 9-4. Once obtained, the lysine-imidazole chelator can be incorporated into a polymer scaffold by procedure similar to that of Scheme 2. An exemplary method is shown in Scheme 10.

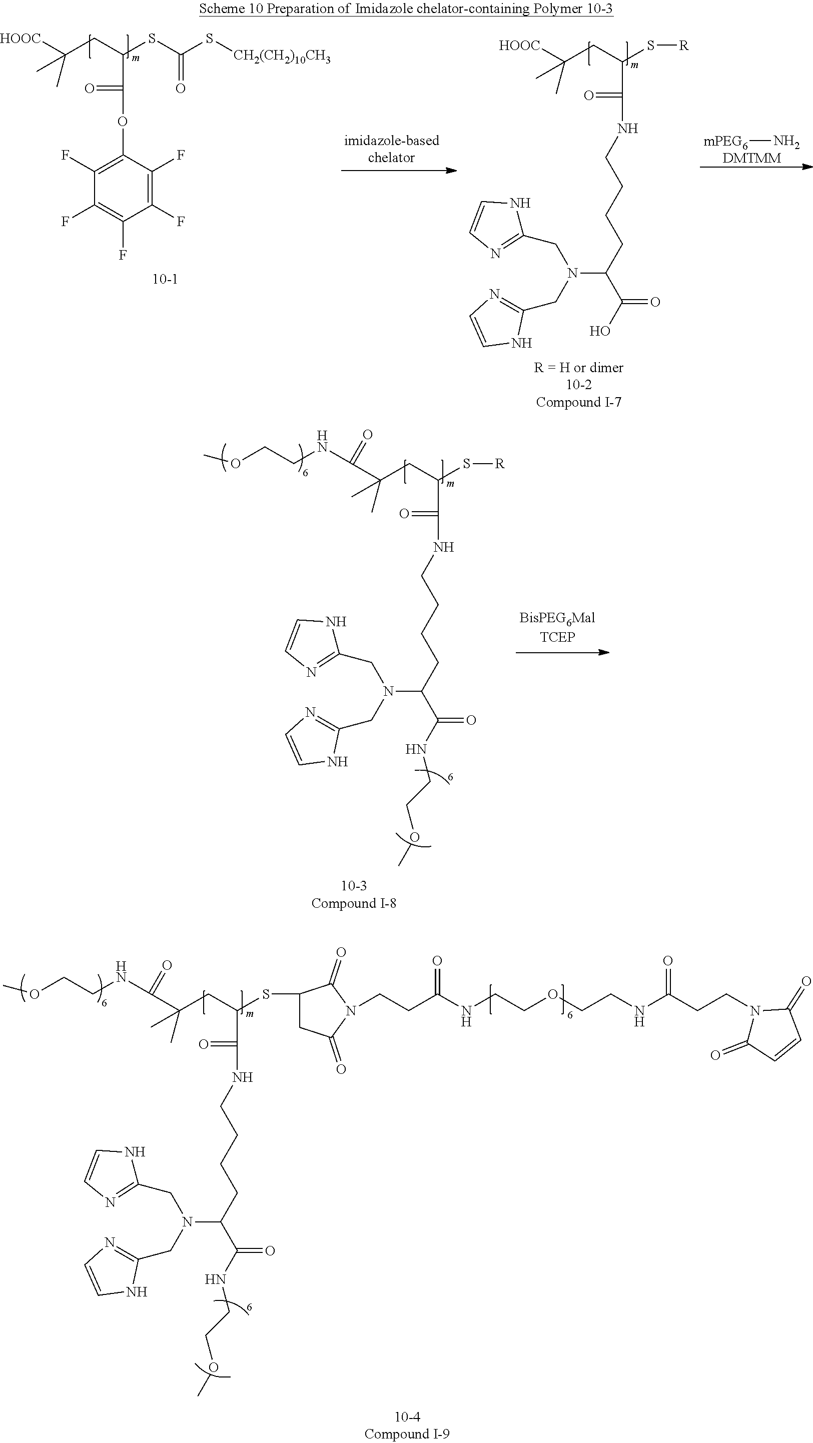

The activated ester polymer 10-1 can be coupled to a lysine-imidazole chelator such as compound 9-4 to obtain a chelating polymer 10-2/Compound 1-7. Optionally, modifying groups such as solubility modifier (e.g. PEG) to obtain polymer 10-3/Compound 1-8. Additionally, reactive functional groups (e.g. maleimide) can be attached to the polymer 10-3 to obtain the polymer 10-4/Compound 1-9.

### Example 9 Preparation of DPA Chelator-Containing Polymers with Zwitterionic Solubility Modifiers

Two exemplary zwitterionic sulfobetaine solubility modifiers were prepared and attached to DPA chelator-containing polymer such as compound 12-1.

3-((3-aminopropyl)-dimethylammonio)propane-1-sulfonate 11-4 was prepared as shown in Scheme 11 based on previously reported synthetic procedure. (26)

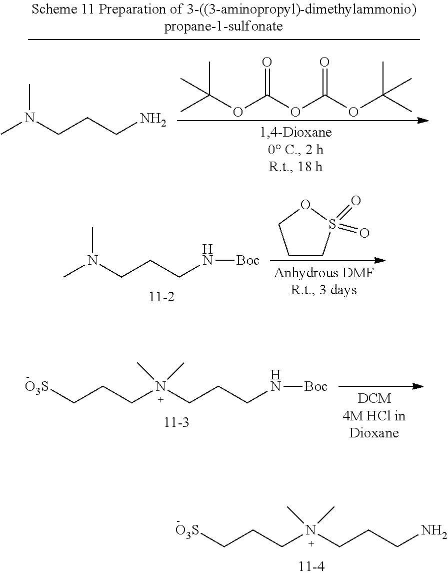

### Compound 11-2:

Di-tert-butyldicarbonate (15.6 g, 71.5 mmol) was added to a solution of 3-(Dimethylamino)-1-propylamine (4.9 g, 6 mL, 47.7 mmol) in 50 mL 1,4-dioxane. The solution was stirred at 0° C. for 2 h, and stirred further overnight at room temperature for 18 h. After 18 h, the solvent was removed in vacuo and 50 mL MilliQ water was added to the crude product. The product was extracted over ethyl acetate (30 mL) three times. The ethyl acetate was dried to afford a slightly yellow, clear oil as compound 11-2. The product was used in the subsequent synthetic steps without further purification.

### Compound 11-3:

Compound 11-2 (1 equiv., 2.5 g, 0.012 mol) was dissolved in 15 mL anhydrous DMF. 1,3-propanesultone (1.4 equiv., 2.113 g, 0.017 mol) was added to the solution of compound 11-2 and stirred for 3 days at room temperature. The crude product was dried in vacuo to remove DMF until a slightly yellow viscous oil remained. The viscous oil was washed over diethyl ether (30 mL), followed by another wash of ethyl acetate (30 mL) to remove remaining 1,3-propanesultone. The oil was freeze-dried to afford a white solid as compound 11-3.

### Compound 11-4:

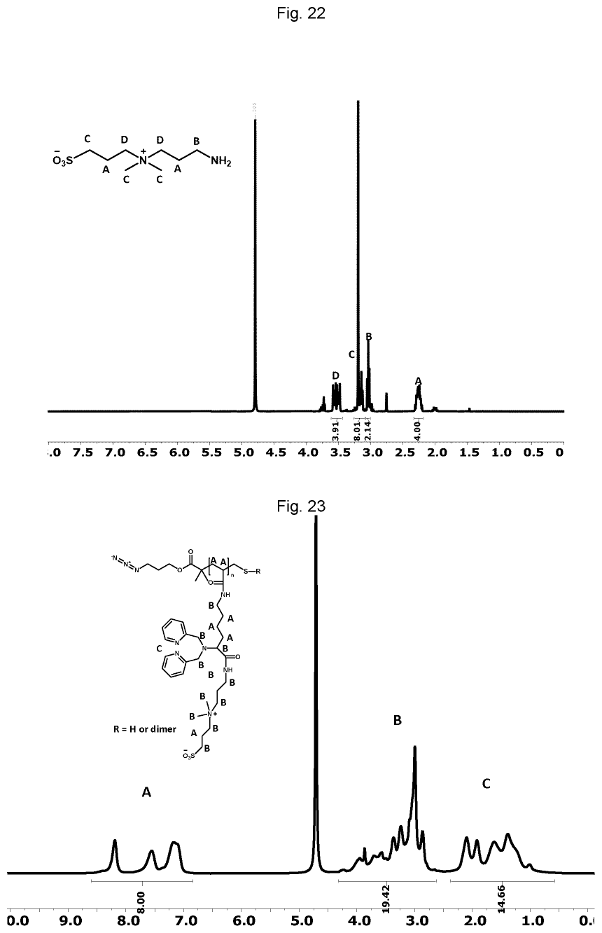

Compound 11-3 (4 g, 0.012 mol) was dissolved in 50 mL DCM, and cooled to 0° C., resulting in a slightly turbid slightly white-coloured solution. After cooling to 0° C., 4 M HCl in dioxane (5 mL) was added to the solution and left to stir for 1 h. After 1 h, the solution became clear, and a solid white chunk precipitate had formed. The solvent was removed in vacuo to dryness and precipitated over DCM/isopropanol/MeOH (10:5:1 v/v ratio). The precipitated product was a gooey white chunk which was freeze-dried. The final product, Compound 11-4 was obtained as a white solid and was stored in the freezer in a vacuum sealed bag. The ¹H NMR spectrum of compound 11-4 is shown on FIG. 22.

### Compound 12-2/I-11

Sulfobetaine 11-4 was then attached to DPA-containing polymeric chelator 12-1 as shown in Scheme 12 to obtain the compound 12-2/polymer 1-11.

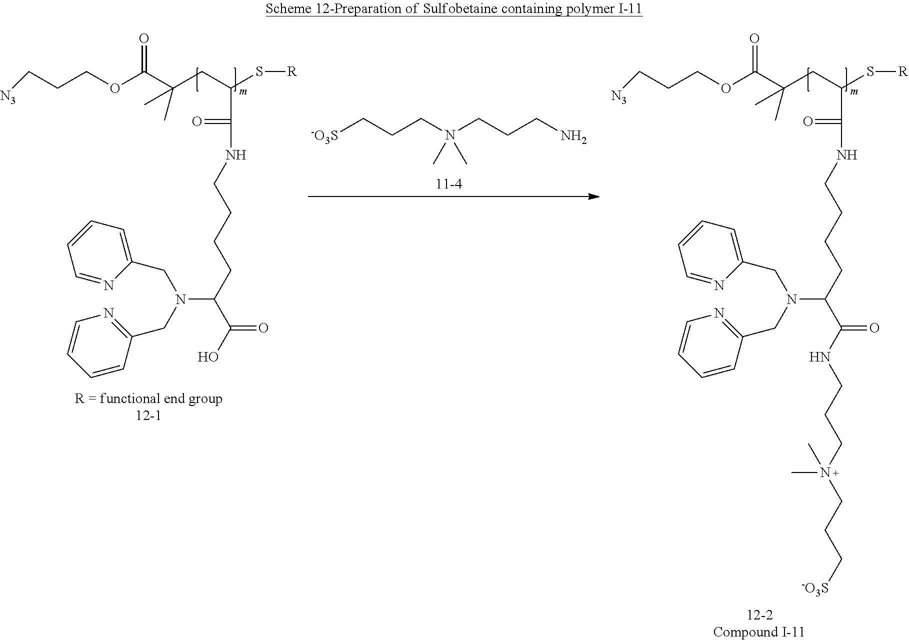

DPA chelator containing polymer 12-1 was dissolved in 0.4 mL 0.2 M pH 8 sodium phosphate buffer. DMTMM (28.8 mg, 0.104 mmol, 7.3 equiv. per pendant group) was dissolved in 0.1 mL MilliQ water and added to the polymer solution and allowed to pre-react for 5 min. After 5 min, sulfobetaine 11-4 (14.58 mg, 0.065 mmol, 5 equiv. per pendant group) was added to the DPA chelator containing polymer 12-1 solution and the reaction mixture was stirred overnight at room temperature for 20 hours. After 20 hours, the reaction mixture was purified using 3 k MWCO Amicon Ultra 4 mL spin filters, washing 5 times with MilliQ water. The retentate was taken and freeze-dried to afford the product, 12-2. The 1H NMR spectrum of the compound 12-2/I-11 is shown in FIG. 23.

### Compound 1-12

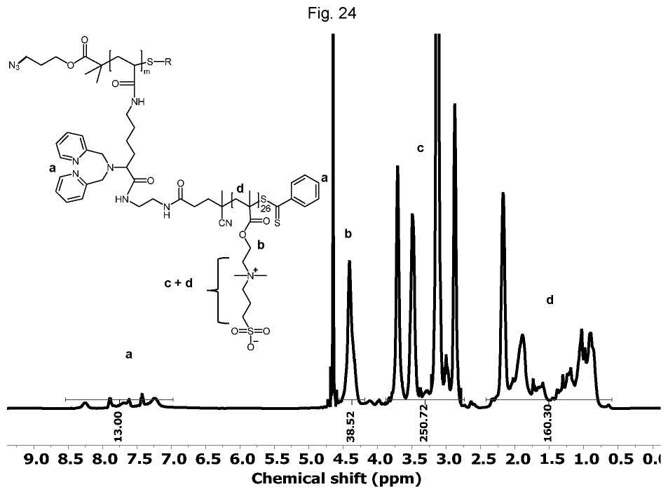

PBSMA 7-1 was prepared as shown in Scheme 7 in Example 7. An ethylene diamine linker was then attached to 7-1 and the resulting compound 13-1 was attached to DPA chelator containing polymer 12-1 to produce polymer 1-12 as shown in Scheme 13. The 1H NMR spectrum of compound I-12 is shown in FIG. 24

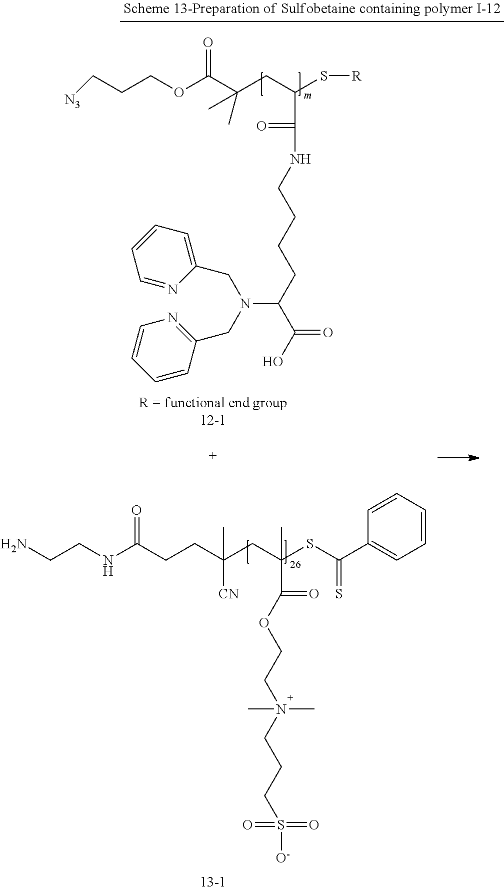

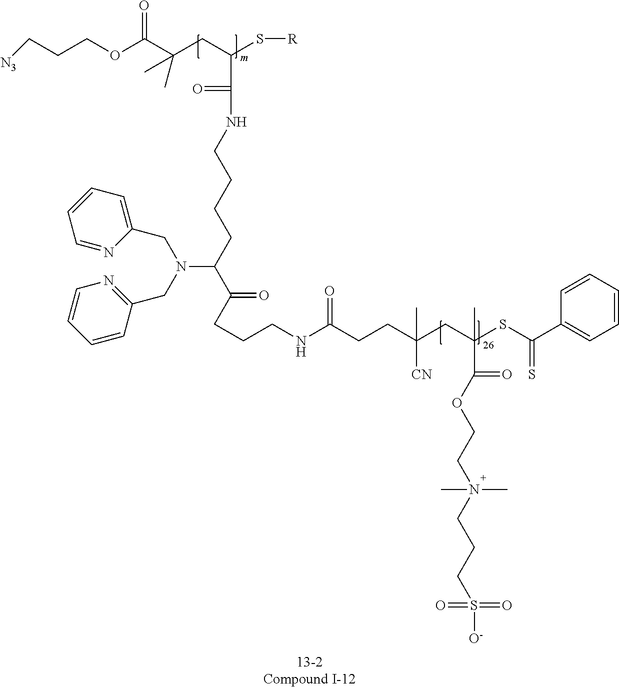

DPA-chelator containing polymer, compound 12-1 (Dp=20, Mₙ=6000 g/mol, 0.0005 mmol, 3 mg) was dissolved in 0.1 mL 0.2 M, pH 8 sodium phosphate buffer. DMTMM (7.3 equiv. per pendant group, 20.20 mg, 0.073 mmol) solution in 0.1 mL MilliQ water was added to the DPA-chelator containing polymer solution and allowed to pre-react for 10 min. Compound 13-1 (Mₙ=5600 g/mol, 112 mg, 0.02 mmol, 2 equiv. per pendant amine) was then added to the pre-reacted DPA-chelator containing polymer, compound 12-1 solution and left to stir overnight at room temperature for ˜20 h. The crude product was purified using Amicon Ultra-4 mL 10 kDa MWCO, spin filters 2× with MilliQ water, 2× with 20 mM NaCl solution, and 3× again with MilliQ water and was freeze-dried to yield the polymer, compound 13-2/I-12.

Alternatively, compound 13-1 can be prepared according to Scheme 13a.

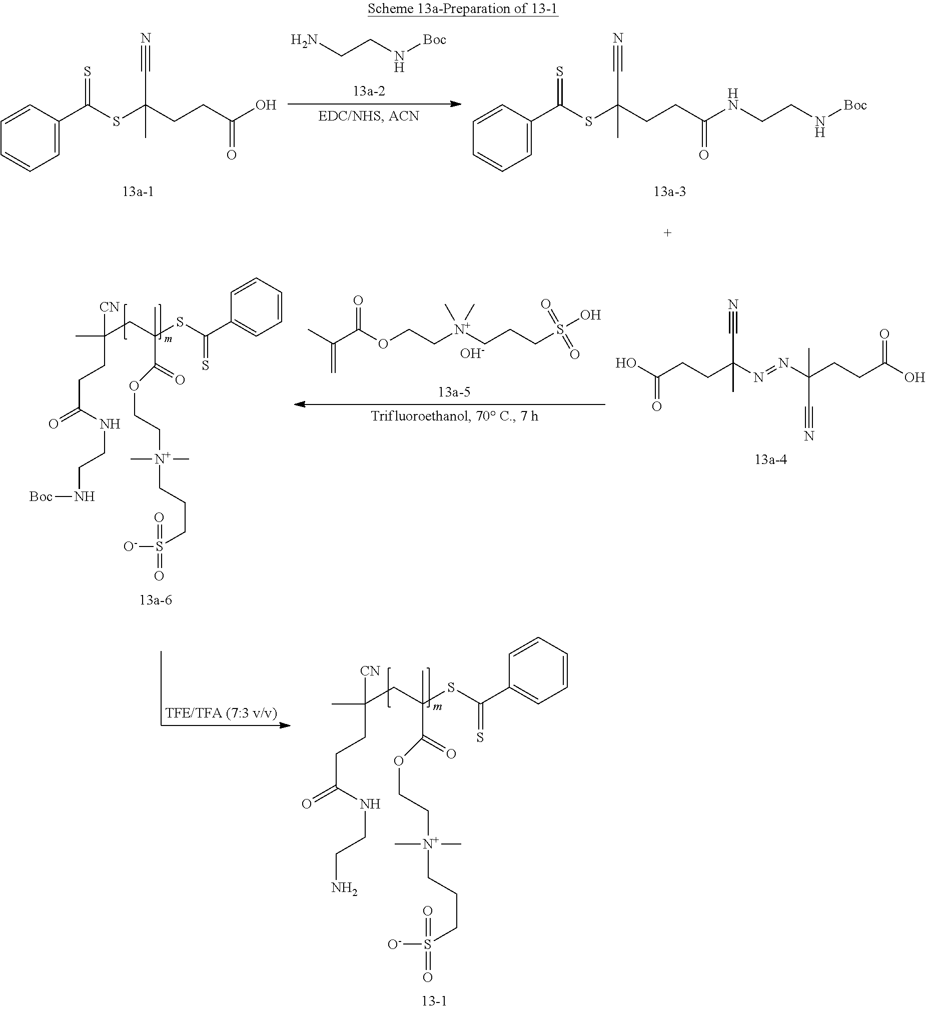

4-Cyano-4-(phenylcarbonothioylthio)pentanoic acid 13a-1 (500 mg, 1.79 mmol, 1 equiv.), EDC (555.8 mg, 3.58 mmol, 2 equiv.), and NHS (412 mg, 3.58 mmol, 2 equiv.) were dissolved in minimal amounts of acetonitrile and vortexed for 15 min. Next, N-Boc-ethylenediamine 13a-2 (430 mg, 2.685 mmol, 1.5 equiv.) was added and stirred overnight After 24 h, the reaction was dried via compressed air and then purified via silica gel flash column chromatography. The solvent system was a gradient elution of 100% DCM to 1:1 DCM/EtOAc. The purified product was dried in vacuo to afford a red-pink solid as the desired product, N-boc CTA 13a-3.

N-boc CTA 13a-3 (45 mg, 0.1067 mmol), SBMA monomer 13a-5 (1.4 g, 2.252 mmol), and 4,4′-Azobis(4-cyanovaleric acid) initiator 13a-4 (3.038 mg, 0.0108 mmol) were dissolved in 4 mL 2,2,2-Trifluoroethanol and the solution was bubbled with nitrogen gas for 20 min. After bubbling the solution with nitrogen gas for 20 min, the reaction was stirred at 70° C. for 6 hours. After 6 h, the reaction was exposed to air, a small aliquot of the crude was taken for H NMR and trifluoroacetic acid (1.7 mL) was added to the remaining crude product and stirred overnight at room temperature to hydrolyze the Boc group. After 21 h, the sample was precipitated over 7:3 diethyl ether/methanol and centrifuged down to a pellet at 2.7 k rcf for 15 min. The supernatant was discarded. The pellet was re-dissolved once over 2,2,2-Trifluoroethanol and re-precipitated over 7:3 diethyl ether/methanol and centrifuged again under the same conditions. This process was repeated two times. The sample was dried in vacuo to afford compound 13-1.

### Example 10 Preparation of Metal-Chelated DPA Chelator-Containing Polymers with Various Solubility Modifiers

Three DPA chelator-containing polymers (Scheme 14) were prepared based on methods described in Examples 3 and 9. Each polymer was metalated with platinum or mercury using K₂PtCl₄ or HgCl₂ respectively. Platinum metalation was carried out in methanol at 45° C. for 2 hours. Mercury metalation was carried out in methanol at room temperature for 1 hour. Successful metalation was assessed by proton NMR by change in chemical shifts of the pyridyl protons. NMR spectra of compounds 14-2/II-7 and 14-3/II-8 are shown in FIG. 21.

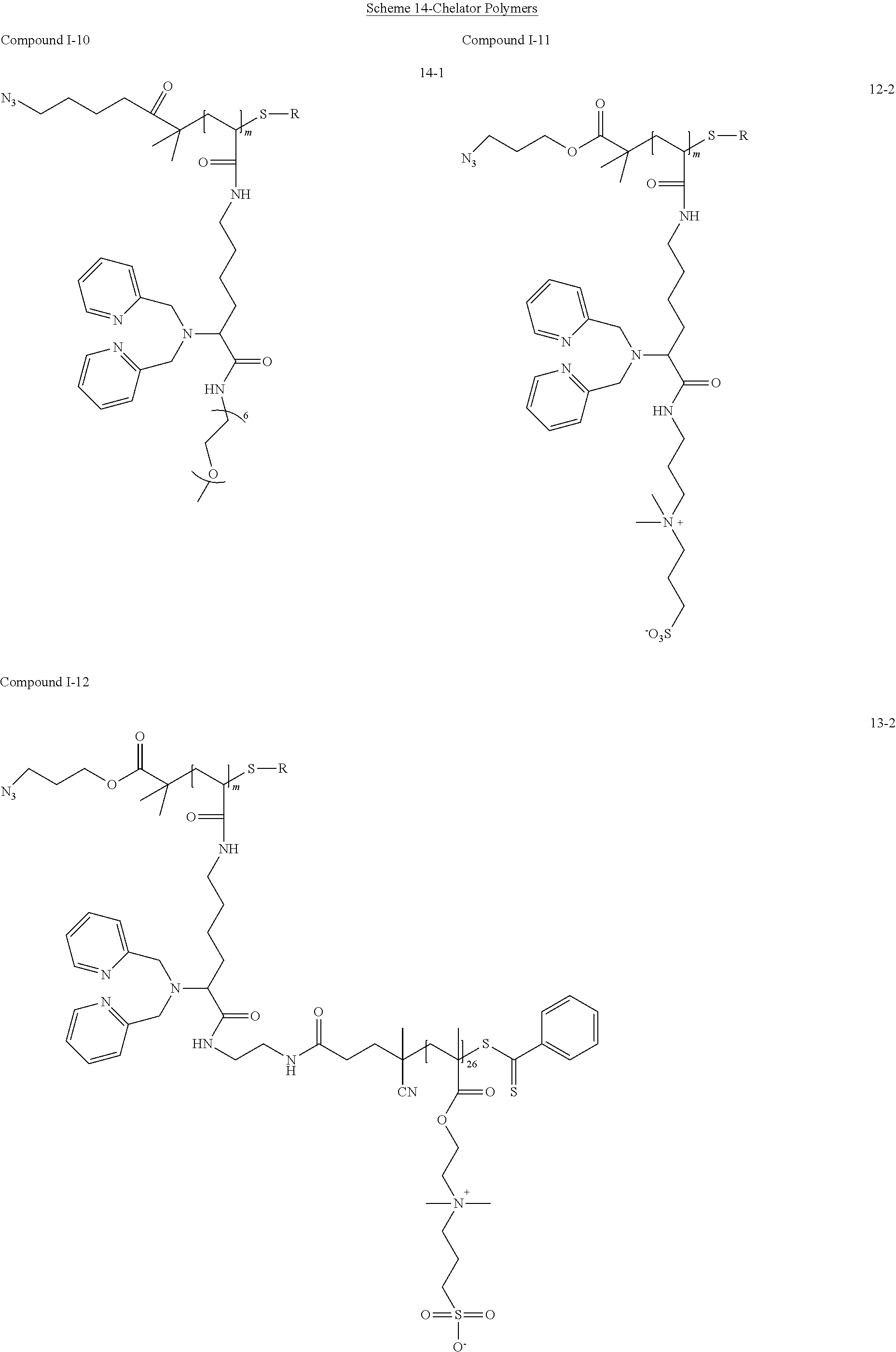

DPA-chelator containing polymer 12-1 (Dp=20, Mₙ=6000 g/mol, 0.0005 mmol, 3 mg) was dissolved in 0.1 mL 0.2 M, pH 8 sodium phosphate buffer. DMTMM (7.3 equiv. per pendant group, 20.20 mg, 0.073 mmol) solution in 0.1 mL MilliQ water was added to the DPA-chelator containing polymer solution and allowed to pre-react for 10 min. Poly(SBMA) 13-1 (Mₙ=5600 g/mol, 112 mg, 0.02 mmol, 2 equiv. per pendant amine) was then added to the pre-reacted DPA-chelator containing polymer, compounds 12-1 solution and left to stir overnight at room temperature for ˜20 h. The crude was purified using Amicon Ultra-4 mL 10 kDa MWCO, spin filters 2× with MilliQ water, 2× with 20 mM NaCl solution, and 3× again with MilliQ water and was freeze-dried to yield the polymer, compound 13-2/I-12.

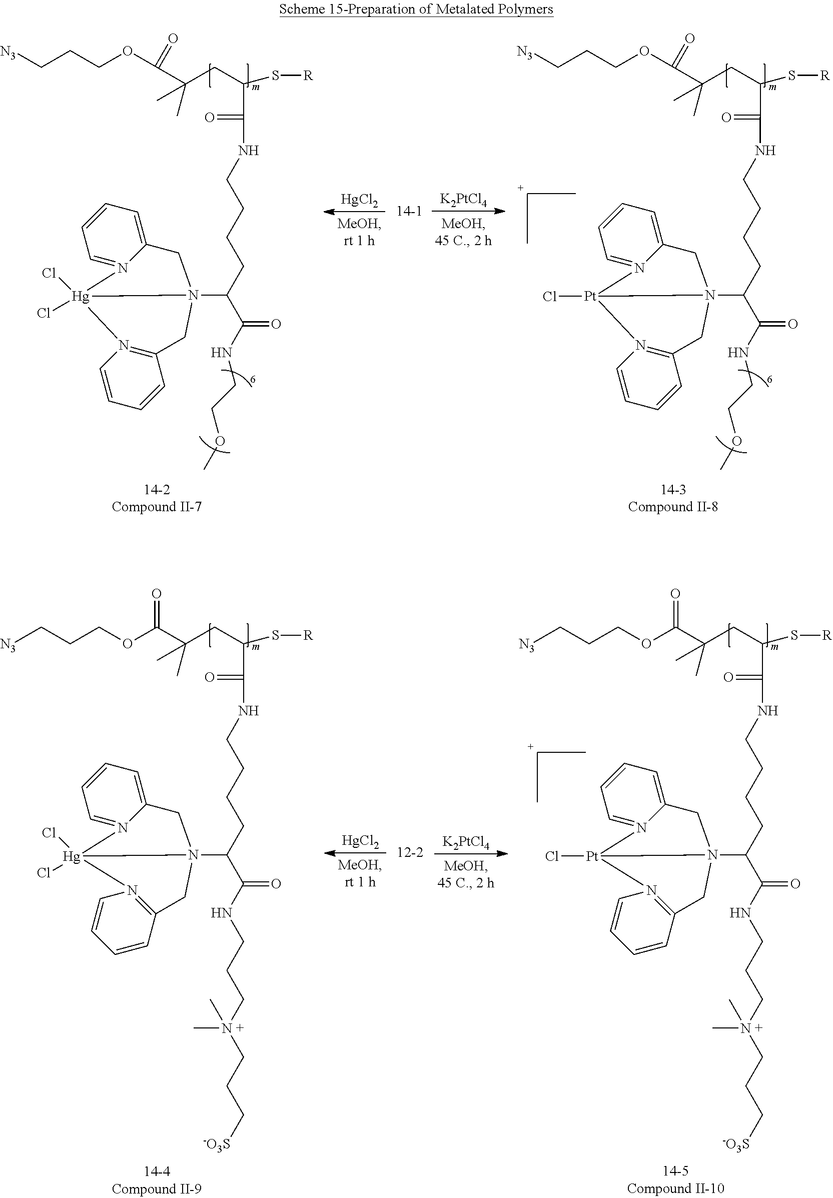

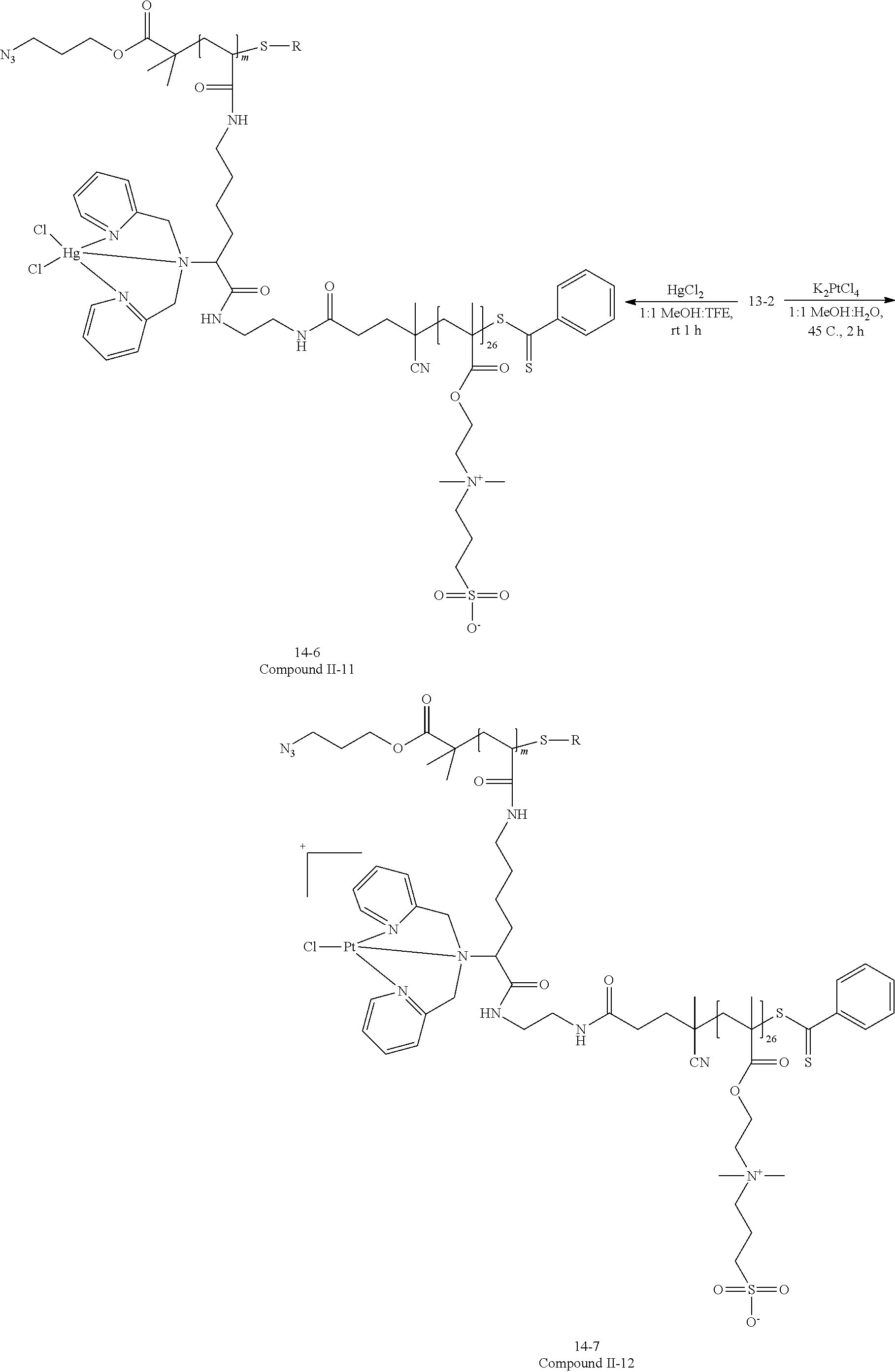

Compound 13-2/I-12 (11.6 mg was dissolved in methanol/2,2,2-Trifluoroethanol (1:1 by volume ratio)). A solution of HgCl₂ (2 mg/mL, 7 mM, 360 mL) was added to the Compound 13-2/I-12 solution and stirred at room temperature. Red precipitates formed within 10 minutes, and the reaction was left to stir at room temperature for 1 h, The red precipitates were pelleted out via centrifugation at 10 k rcf for 10 minutes. The supernatant was discarded and the pellet was further washed 2× with MeOH. The red pellet was freeze-dried to afford compound 14-6/II-11. (methodology adapted from 27)

Compound 13-2/I-12 (20 mg was dissolved in Aqueous MeOH (1:1 by volume ratio)). A solution of K₂PtCl₄ (50 mM, 360 mL) was added to compound 13-2/I-12 and was then heated at 45° C. for 2 h, resulting in a yellow solution. The yellow solution was spin filtered over Amicon Ultra-15 mL 10 k MWCO spin filters 2× with MilliQ water, 3× with 20 mM NaCl solution, and one wash with MilliQ water. The sample was then freeze-dried to afford compound 14-7/II-12.

The metalation reaction can also be done with 2,2,2-Trifluoroethanol and MeOH/2,2,2-Trifluoroethanol as the solvent system.

### Example 11 Mass Cytometry Tests of Rhenium-Containing Antibody-Conjugated Polymers with Zwitterionic Solubility Modifier

Rhenium-chelated polymers of the present disclosure comprising zwitterionic solubility modifiers were conjugated to antibodies and assessed for non-specific binding using mass cytometry.

### Antibody Conjugation

To evaluate the performance of the rhenium-tagged polymer with zwitterionic solubility modifier in mass cytometry immunoassays, two primary antibodies, CD20 and CD8a, were labeled with the polymer tag following a revised Maxpar™ antibody labeling protocol. Briefly, the antibody was partially reduced by TCEP, washed in a spin filter (30 kDa), and then mixed with an excess (20-fold) of DBCO-PEG₄-maleimide. The mixture was incubated at 37° C. for 30 min. The DBCO modified Abs were purified using a spin filter (30 kDa) and then mixed with desired polymer mass tags. The mixture was incubated at 37° C. for 90 min. Excess polymers were removed by a spin filter (100 kDa).

### Mass Cytometry Titration Experiments

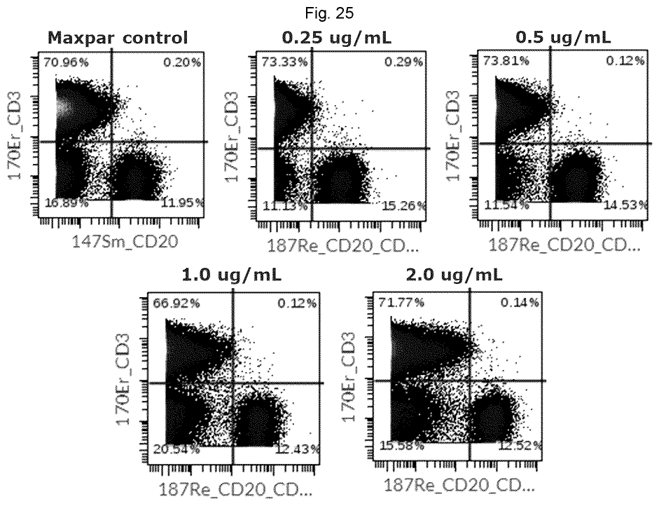

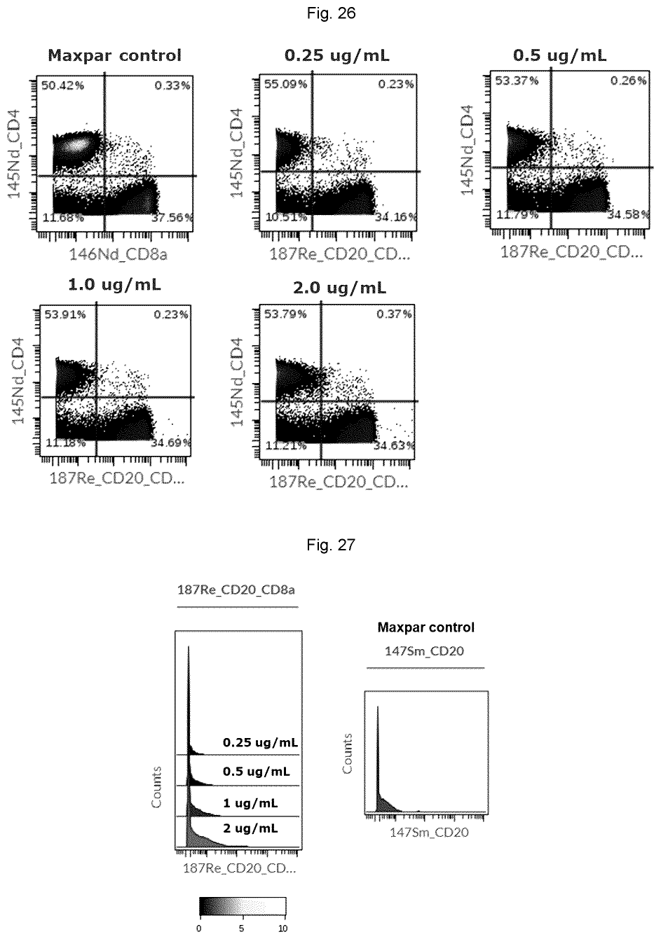

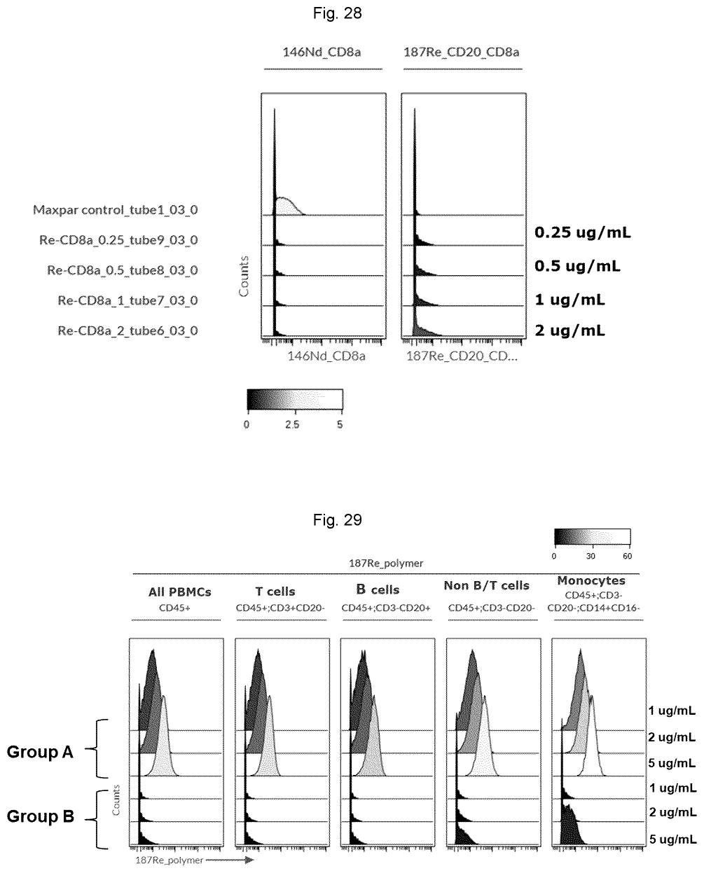

Antibody titration experiments were then performed with human peripheral blood mononuclear cells (PBMCs) to evaluate the performance of the conjugate. In these experiments, human PBMCs were stained with a 7-plex antibody panel (Fluidigm Maxpar™ reagents), including ¹⁴⁵Nd-CD4, ¹⁴⁶Nd-CD8a, ¹⁶⁵Ho-CD16, ¹⁵⁴Sm-CD45, ¹⁶⁰Gd-CD14, ¹⁷⁰Er-CD3 and ¹⁴⁷Sm-CD20. For the titration of rhenium conjugates, separate staining panels, where ¹⁴⁷Sm-CD20 and ¹⁴⁶Nd-CD8a were replaced with ^{187/185}Re-CD20 and ^{187/185}Re-CD8a, were used. ^{187/185}Re-CD20/CD8a conjugates were titrated at concentrations of 0.25, 0.5, 1 and 2.0 μg/mL. As shown in FIG. 25, ^{187/185}Re-CD20 allows distinct separation of CD20+B-cell subsets from the rest of cell subsets in PBMCs. Similarly, ^{187/185}Re-CD8a also allows distinct separation of CD8+ T-cell subsets from the rest of cell subsets in PBMCs at all titers (FIG. 26). Importantly, both conjugates showed minimal non-specific binding to other cell populations as shown in FIGS. 27 and 28. The rhenium-tagged CD20 conjugate showed minimal non-specific binding to non-T/B cells. (FIG. 27) The rhenium-tagged CD8a conjugate showed minimal non-specific binding to B cells. (FIG. 28)

Comparison with PEG Modified Re-Containing Polymers

To assess non-specific binding properties of the zwitterionic solubility modifier compared to other solubility modifiers, PEG modified polymers were synthesized and chelated to Re metal based on methods described in Example 1. For mass cytometry experiments, both polymers were mixed with the antibody staining cocktails at various concentrations (1, 2 and 5 ug/mL) and the resulting antibody cocktails were used to stain PBMCs following regular staining protocol as described above. It was observed that this PEG modified polymer exhibited non-specific binding to major cell subsets within PBMCs at 1 ug/mL. (see FIG. 29)

Polymers of the present disclosure modified with PEG solubility modifier showed higher non-specific binding to PBMCs compared with polymers modified with zwitterionic solubility modifier. (see FIG. 29) Polymers modified with zwitterionic solubility modifier showed minimal non-specific binding to major subsets of PBMCs at 5 ug/mL. The zwitterion modified rhenium polymer showed comparably less non-specific binding to PBMCs than that of PEG modified rhenium polymer.

### Example 12 Preparation of H-Dap Dipicolylamine Chelator

H-Dap dipicolylamine based chelator was prepared as shown in Scheme 16.

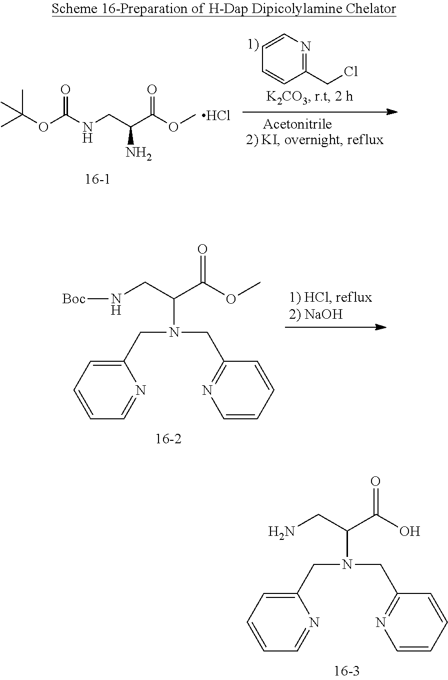 [SMILES: ClCc1ccccn1 ; COC(=O)[C@@H](N)CNC(=O)OC(C)(C)C ; Cl ; COC(=O)C(CNC(=O)OC(C)(C)C)N(Cc1ccccn1)Cc1ccccn1 ; NCC(C(=O)O)N(Cc1ccccn1)Cc1ccccn1]

H-Dap (Boc)-OMe HCl 16-1 (0.5 g, 1.86 mmol, 1 equiv.) was dissolved in ˜30 mL anhydrous acetonitrile and bubbled with N₂(g) with stirring for 30 minutes. Next, 2-picolyl chloride hydrochloride (2.2 equiv., 671.21 mg, 4.092 mmol), K₂CO₃ (3.2 equiv, 5.95 mmol, 822.6 mg) were successively added and the reaction was stirred at r.t. for 2 h. After stirring for 2 hours, potassium iodide (1 equiv., 679.27 mg, 4.092 mmol) was added and the reaction was heated and stirred at reflux (−85° C.). The reaction was then left to react overnight (˜18 h). The sample was dried in vacuo to remove acetonitrile. The dried crude product was re-dissolved in DCM. The DCM organic layer was washed with water 3× 100 mL. The organic layer was collected and dried in vacuo to afford the product, H-Dap Boc-OMe dipicolylamine 16-2 as a brown solid.

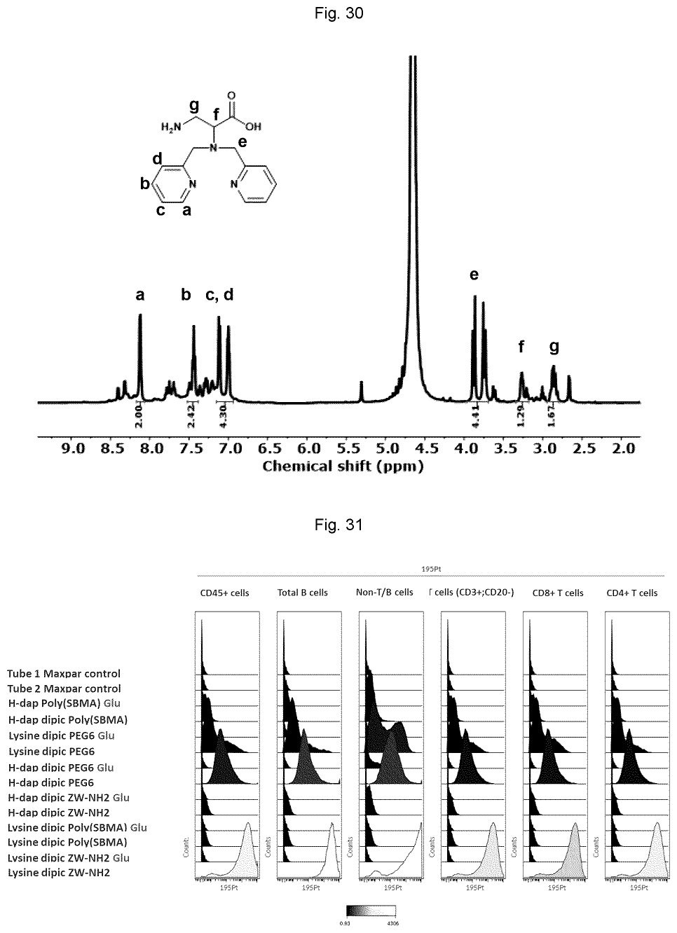

H-Dap Boc-OMe dipicolcylamine 16-2 (185 mg) was dissolved in a 40 mL 1:1 mixture of MiIIIQ water and concentrated HCl (to make ˜6M HCl). The sample was then refluxed overnight at 110° C. (Reaction start time: 11:40 am). After 24 h, the reaction mixture was dried in vacuo. The sample was re-dissolved in ˜20 mL MilliQ water and basified over 1 M NaOH to neutralize the acid (checked with pH paper). The sample freeze dried overnight. and then re-dissolved over DCM. The DCM layer was then centrifuged at 2.7 k rcf 15 min to remove any salts from the neutralization. The product 16-3 was dried and collected as a dark brown solid. The 1H NMR spectrum of compound 16-3 is shown in FIG. 30.

The chelator 16-3 was used in the preparation of the polymers of the present disclosure as described herein.

### Example 13 Addition of Modifiers to Metal-Containing Polymers Through Ligand Exchange

Modifiers such as solubility modifiers have been installed on metal-containing polymers via the metal centre by ligand exchange with small molecules such as glutathione. (Schemes 17 and 18)

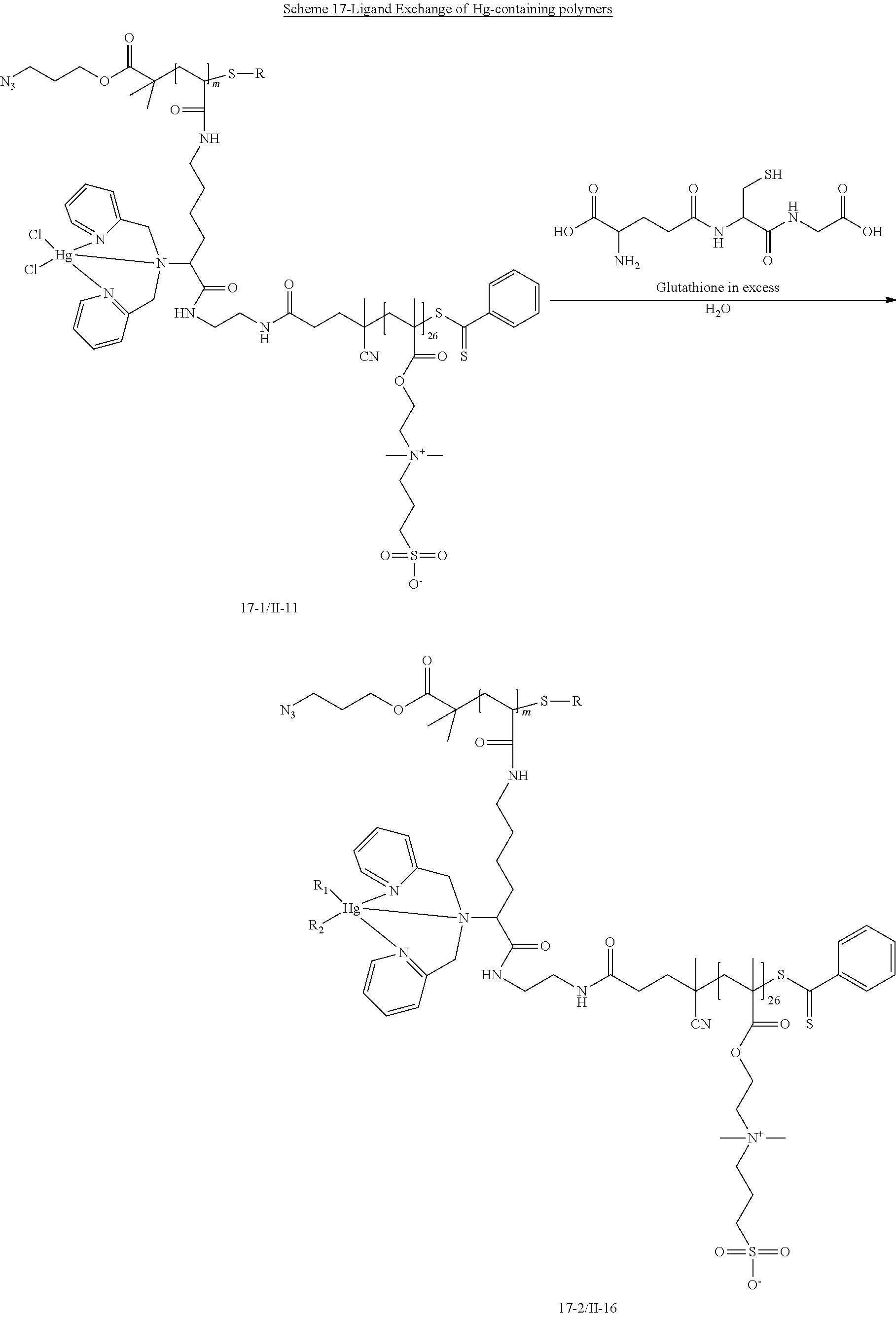

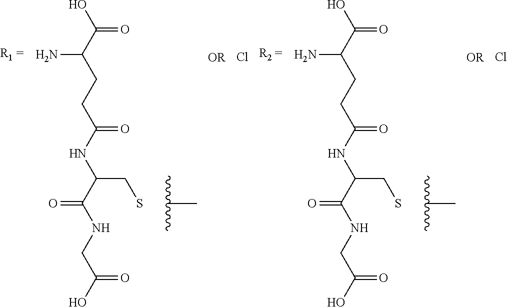

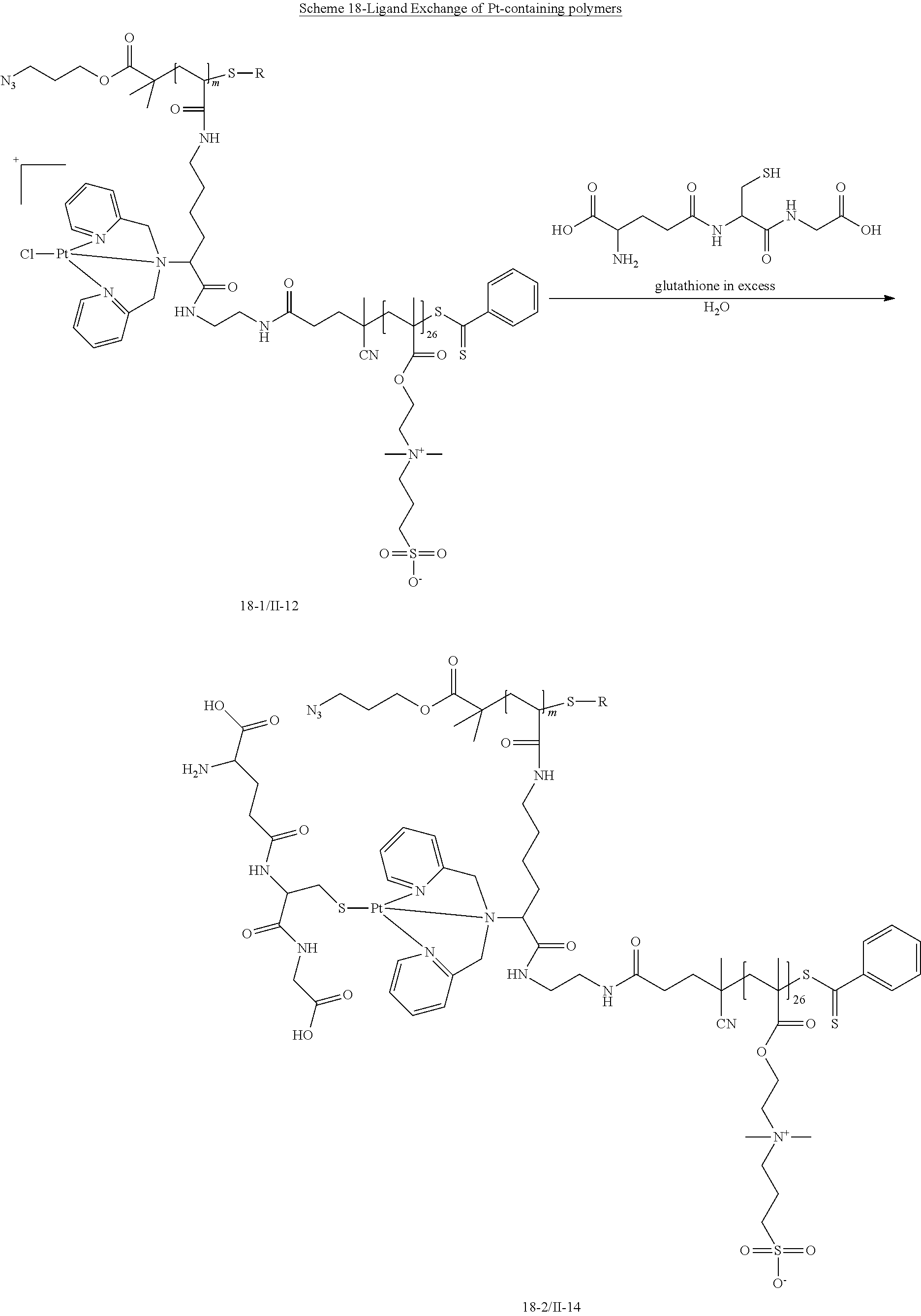

To a solution containing mercury-containing Polymer or Platinum-containing Polymer (3 mg in 250 uL) glutathione solution (325 mM, 50 uL) was added and the reaction was vortexed gently for 15 minutes at room temperature. The samples were then spin filtered over Amicon, Ultra-0.5, 3 kDa at 2.7 k rcf with MilliQ water 8 times. The samples were freeze-dried to afford the polymers.

Other examples of small molecule thiols containing one or more thiol functional groups for ligand exchange reactions described here include but are not limited to cysteine, thioglycolic acid, mercaptosuccinic acid, methyl thioglycolate, dimercaprol, dimercaptosuccinic acid, 2,3-dimercapto-1-propanesulfonate.

It was observed that polymers containing glutathione ligands were more soluble compared to the original polymers with halide ligands. For example, for the Hg polymers, any undissolved species were observed in MilliQ water. These precipitates were filtered out using 0.2 um nylon syringe filters. Addition of glutathione was able to re-dissolve the precipitate after which they were purified by spin filtration using Amicon, Ultra-0.5, 3 kDa spin filters. ¹H NMR showed that the re-dissolved (in water) species were the DPA-sulfobetaine modified polymers. Accordingly, ligand exchange to install solubility modifier at the metal centre was able to increase solubility.

Several glutathione modified Pt-containing polymers of the present disclosure were prepared and compared to their counterpart without glutathione ligand as a solubility modifier:

TABLE 2
Metal-containing polymers with or without glutathione ligand

| | | Glutathione | |
|---|---|---|---|
| Chelator | Solubility modifier | ligand (Y/N) | Compound number |
| H-dap dipic | PSBMA | Y | |
| H-dap dipic | PSBMA | N | |
| Lysine dipic | PEG6 | Y | II-13 (14-3/II-8 undergone ligand |
| | | | exchange with glutathione) |
| Lysine dipic | PEG6 | N | 14-3/II-8 |
| H-dap dipic | PEG6 | Y | |
| H-dap dipic | PEG6 | N | |
| H-dap dipic | Zwitterionic-NH₂ | Y | |
| H-dap dipic | Zwitterionic-NH₂ | N | |
| Lysine dipic | PSBMA | Y | II-14 (14-7/II-12 undergone ligand |
| | | | exchange with glutathione) |
| Lysine dipic | PSBMA | N | 14-7/II-12 |
| Lysine dipic | Zwitterionic-NH₂ | Y | II-15 (14-5/II-10 undergone ligand |
| | | | exchange with glutathione) |
| Lysine dipic | Zwitterionic-NH₂ | N | 14-5/II-10 |

The polymers were conjugated to antibodies and assessed for non-specific binding using mass cytometry methods based on those described in Example 11. The results are shown in FIG. 31. As shown in the mass cytometry results, glutathione modified polymers exhibited lower non-specific binding for all polymers tested.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### CITATIONS FOR REFERENCES REFERRED TO IN THE SPECIFICATION

(1) Manners, I. Synthetic metal-containing polymers; John Wiley & Sons, 2006.
(2) Abd-El-Aziz, A. S.; Manners, I. Frontiers in transition metal-containing polymers; John Wiley: Hoboken, N.J., 2007.
(3) Abd-El-Aziz, A. S.; Todd, E. K. Coordination Chemistry Reviews 2003, 246, 3-52.
(4) Chan, W. K. Coordination Chemistry Reviews 2007, 251, 2104-2118.
(5) Happ, B.; Winter, A.; Hager, M. D.; Schubert, U. S. Chem. Soc. Rev. 2012, 41, 2222-2255.
(6) Wang, X.; McHale, R. Macromolecular Rapid Communications 2010, 31, 331-350.
(7) Yan, Y.; Zhang, J.; Ren, L.; Tang, C. Chem. Soc. Rev. 2016, 45, 5232-5263.
(8) Bandura, D. R.; Baranov, V. I.; Ornatsky, O. I.; Antonov, A.; Kinach, R.; Lou, X. D.; Pavlov, S.; Vorobiev, S.; Dick, J. E.; Tanner, S. D. Anal. Chem. 2009, 81, 6813-6822.
(9) Illy, N.; Majonis, D.; Herrera, I.; Omatsky, O.; Winnik, M. A. Biomacromolecules 2012, 13, 2359-2369.
(10) Lou, X. D.; Zhang, G. H.; Herrera, I.; Kinach, R.; Ornatsky, O.; Baranov, V.; Nitz, M.; Winnik, M. A. Angewandte Chemie International Edition 2007, 46, 6111-6114.
(11) Majonis, D.; Herrera, I.; Ornatsky, O.; Schulze, M.; Lou, X. D.; Soleimani, M.; Nitz, M.; Winnik, M. A. Anal. Chem. 2010, 82, 8961-8969.
(12) Han, G.; Chen, S. Y.; Gonzalez, V. D.; Zunder, E. R.; Fantl, W. J.; Nolan, G. P. Cytom. A 2017, 91A, 1150-1163.
(13) Han, G. J.; Spitzer, M. H.; Bendall, S. C.; Fantl, W. J.; Nolan, G. P. Nat. Protoc. 2018, 13, 2121-2148.
(14) Majonis, D.; Ornatsky, O.; Kinach, R.; Winnik, M. A. Biomacromolecules 2011, 12, 3997-4010.
(15) de Rosales, R. T. M.; Finucane, C.; Foster, J.; Mather, S. J.; Blower, P. J. Bioconjugate Chem. 2010, 21, 811-815.
(16) Duatti, A. Nuclear Medicine and Biology 2021, 92, 202-216.
(17) Ranasinghe, K.; Handunnetti, S.; Perera, I. C.; Perera, T. Chemistry Central Journal 2016, 10.
(18) Storr, T.; Fisher, C. L.; Mikata, Y.; Yano, S.; Adam, M. J.; Orvig, C. Dalton Trans. 2005, 654-655.
(19) Fernandez-Moreira, V.; Thorp-Greenwood, F. L.; Coogan, M. P. Chem. Commun. 2010, 46, 186-202.
(20) Levadala, M. K.; Banerjee, S. R.; Maresca, K. P.; Babich, J. W.; Zubieta, J. Synthesis-Stuttgart 2004, 1759-1766.
(21) Bou Zerdan, R.; Geng, Z.; Narupai, B.; Diaz, Y. J.; Bates, M. W.; Laitar, D. S.; Souvagya, B.; Van Dyk, A. K.; Hawker, C. J. Journal of Polymer Science 2020, 58, 1989-1997.
(22) Jochum, F. D.; Theato, P. Macromolecules 2009, 42, 5941-5945.
(23) Boyle, A. J.; Liu, P.; Lu, Y.; Weinrich, D.; Scollard, D. A.; Mbong, G. N. N.; Winnik, M. A.; Reilly, R. M. Pharmaceutical Research 2013, 30, 104-116.
(24) Alberto, R.; Egli, A.; Abram, U.; Hegetschweiler, K.; Gramlich, V.; Schubiger, P. A. Journal of the Chemical Society-Dalton Transactions 1994, 2815-2820.
(25) Deygen, I. M.; Kudryashova, E. V. Colloids and Surfaces B-Biointerfaces 2016, 141, 36-43.
(26) Wang, W.; Ji, X.; Kapur, A.; Zhang, C.; Mattoussi, H. A Multifunctional Polymer Combining the Imidazole and Zwitterion Motifs as a Biocompatible Compact Coating for Quantum Dots. J. Am. Chem. Soc. 2015, 137, 14158-14172.
(27) Ye, Z-Y, Zhang Z-Y, Huo L-H, Deng, Z-P, Zhang X-F, Gao S, Polyhedron, 2016, 338-351.

1. A compound of Formula I 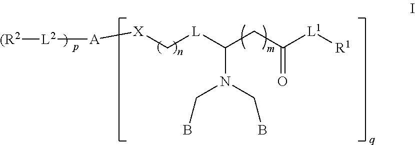
wherein
A is a polymer backbone, optionally, the polymer is a linear polymer, branched polymer, hyperbranched polymer, co-polymer, or combinations thereof;
each B is independently a nitrogen-containing 5-membered to 7-membered heterocycle, optionally substituted with one or more polar functional groups selected from C1-C6 COOH, C1-C6 alkoxy, C1-C6 alkyl phosphonate, alkyl ether, polyether, or combinations thereof; and
L is absent or a linker;
each L² is independently absent or a linker;
each R² is independently a first modifying group selected from a solubility modifier, a reactive functional group, a biomolecule, or combinations thereof;
X is a functional group selected from ester, ether and amide;
each L¹ is independently absent or a linker;
each R¹ is independently H, C1 to C8 alkyl, C2 to C8 alkenyl, C3-C8 cycloalkyl, OH, C1 to C10 alkoxy, C1 to C10 alkyl amine, a solubility modifier, a reactive functional group, a biomolecule and combinations thereof;
n is an integer from 0 to 7;
m is an integer from 0 to 4;
p is an integer from 0 to 3; and
q is an integer above 0.

2. The compound of claim 1, wherein each B is independently a nitrogen-containing 5-membered or 6-membered heteroaryl, optionally wherein each B is independently a pyridine or imidazole, optionally substituted with one or more polar functional groups selected from COOH, C1-C6 alkoxy, C1-C6 alkyl phosphonate, alkyl ether, polyether, or combinations thereof, and wherein optionally one or more B are coordinated to a soft metal, and/or conjugated to one or more biomolecules.

3. The compound of claim 1 or 2, wherein one or more B are coordinated to a soft metal, optionally selected from Re, Pt, Pd, Nb, Tc, Hg, Ag, Au, Mo, Ru, Rh, Cd, W, Os, or mixtures thereof.

4. The compound of any one of claims 1 to 3, wherein R¹ or R² is the biomolecule, optionally the biomolecule is an affinity reagent, such as an antibody.

5. The compound of any one of claims 1 to 4, wherein X is —C(O)NR⁴— or —NR⁴C(O)—, wherein R⁴ is H or C1 to C4 alkyl.

6. The compound of any one of claims 1 to 5, wherein X is —C(O)NR⁴— and the compound has a structure of Formula Ia 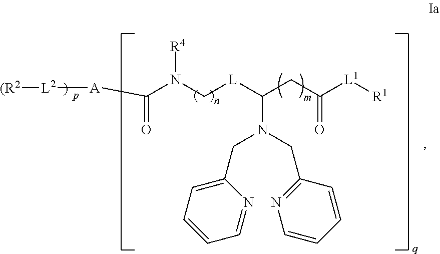
or wherein X is —NR⁴C(O)— and the compound has a structure of Formula Ib

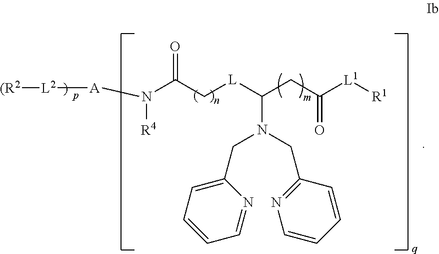

7. The compound of any one of claims 1 to 5, wherein X is —C(O)NR⁴— and the compound has a structure of Formula Ic 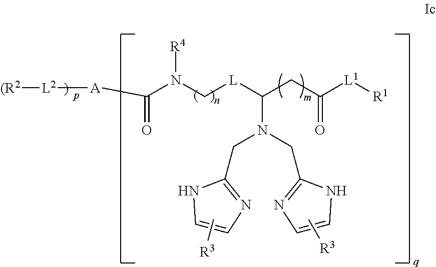
or, wherein wherein X is —NR⁴C(O)— and the compound has a structure of Formula If 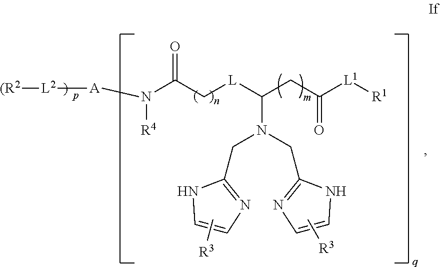
or wherein the compound has a structure of Formula Id or Ie 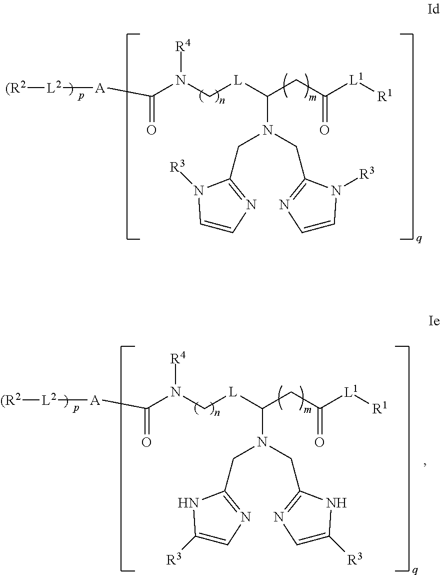
or wherein the compound has a structure of Formula Ig or Ih 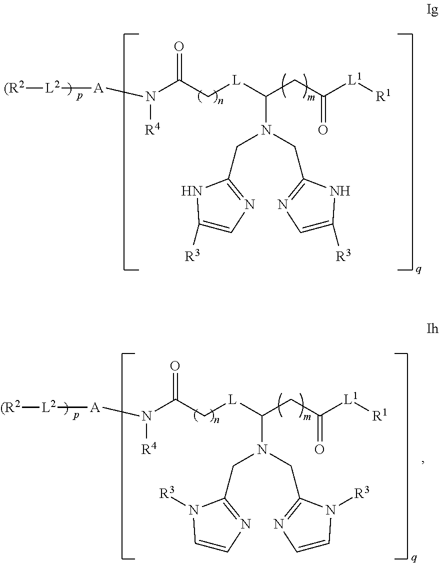
wherein each R³ is independently selected from H, C1 to C5 alkyl, C2 to C5 alkenyl, C1-C6 COOH, C1-C6 alkoxy, C1-C6 alkyl phosphonate, alkyl ether, or polyether.

8. The compound of claim 7 or 8, wherein each R³ is independently selected from H, —(CH₂)₁₋₃COOH, —(CH₂)₁₋₃O(CH₂)₁₋₂CH₃, —(CH₂)₂₋₄OH, —(CH₂)₂₋₅P(O)(OCH₂CH₃)₂, or —CH₂CH(OMe)₂, and/or wherein n is 2, 3, 4, or 5, wherein m is 0, 1, or 2, or wherein p is 1 or 2.

9. The compound of any one of claims 1 to 8, wherein A is selected from polyacrylate, polyacrylamide, polyether, polyamino acid, polyvinyl amine, poly(2-oxazoline), polyethylene glycol, polysaccharide, dendrimer, co-polymers thereof, or combinations thereof.

10. The compound of any one of claims 1 to 9, wherein each linker independently comprises or is independently selected from C3-C8 alkyl amine, C3-C8 alkylene, C3-C8 cycloalkyl, C3-C8 heterocycloalkyl, 5-membered or 6-membered aryl or heteroaryl, alkylaryl, alkylheteroaryl, C3-C8 cycloalkylaryl, C3-C8 cycloalkylheteroaryl, C(O), C(O)O, amide, amine, thioether, maleimide-thiol conjugate, polyethylene glycol (PEG), or mixtures thereof, optionally each of the amine, alkylene, aryl, alkylaryl, alkylheteroaryl, cycloalkyl, cycloalkylaryl, and cycloalkylheteroaryl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

11. The compound of any one of claims 1 to 10, wherein each L¹ and/or L² independently comprises or is independently selected from C3-C8 alkylene, C3-C8 alkyl amine, ester, amine, amide, thioether, maleimide-thiol conjugate, PEG, or mixtures thereof, optionally each of the alkylene and alkyl is independently unsubstituted or substituted with one or more substituents selected from C1-C6 alkyl, C1-C6 alkenyl, C3-C8 cycloalkyl, C3-C8 heterocycloalkyl, amide, ester, aryl, heteroaryl, alkylaryl, alkylheteroaryl, C3-C8 cycloalkylaryl, C3-C8 cycloalkylheteroaryl, CN, or mixtures thereof.

12. The compound of any one of claims 1 to 11, wherein the solubility modifier of the first modifying group of each R² and the solubility modifier of the second modifying group each independently comprises polyethyleneglycol (PEG), sugar, oligosaccharide, or zwitterionic polymer such as poly(carboxylbetaine) methacrylate or poly(sulfobetaine) methacrylate (PBSMA).

13. The compound of any one of claims 1 to 12, wherein the reactive functional group is for attachment to one or more biomolecules, optionally the one or more biomolecules being each independently selected from a small molecule, a polypeptide, an oligonucleotide, a lipid, a carbohydrate, affinity reagent, optionally an antibody, or a mixture thereof.

14. The compound of claim 1, wherein the compound is selected from 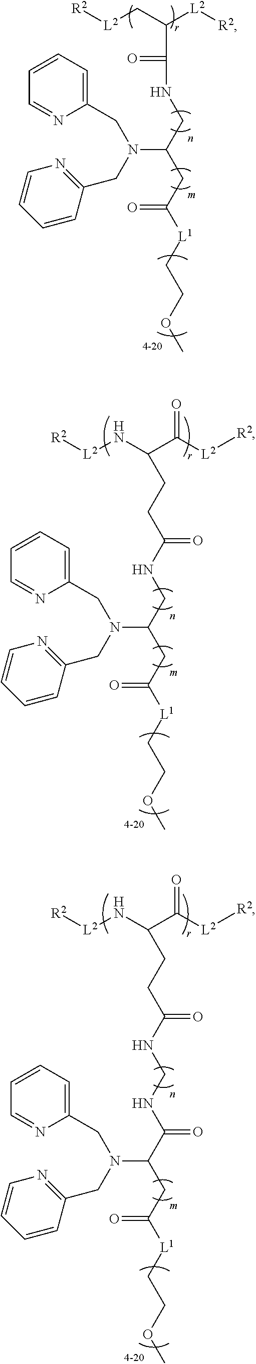 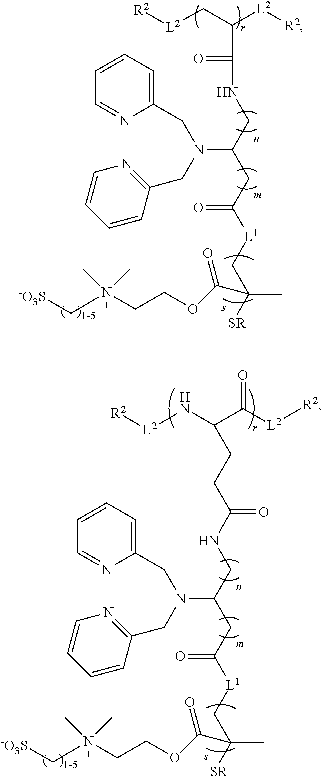 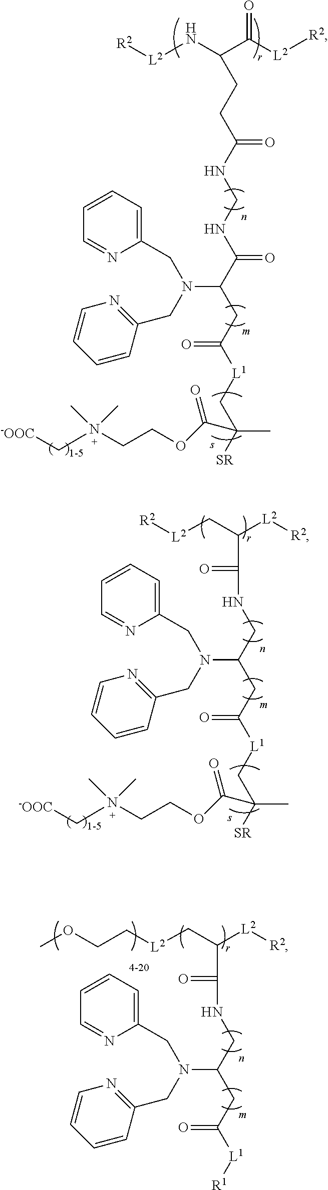 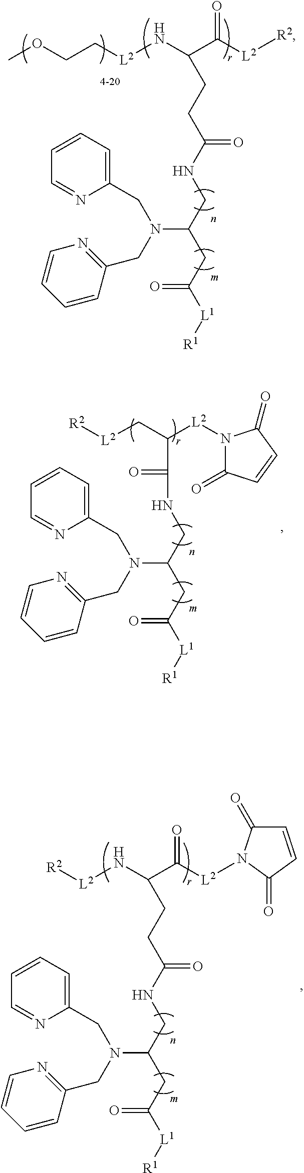 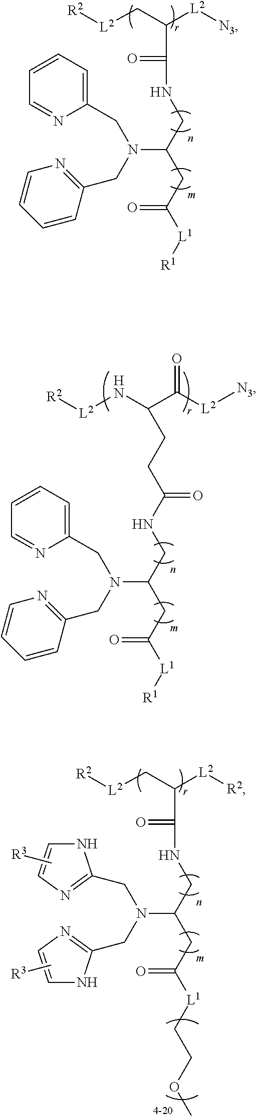 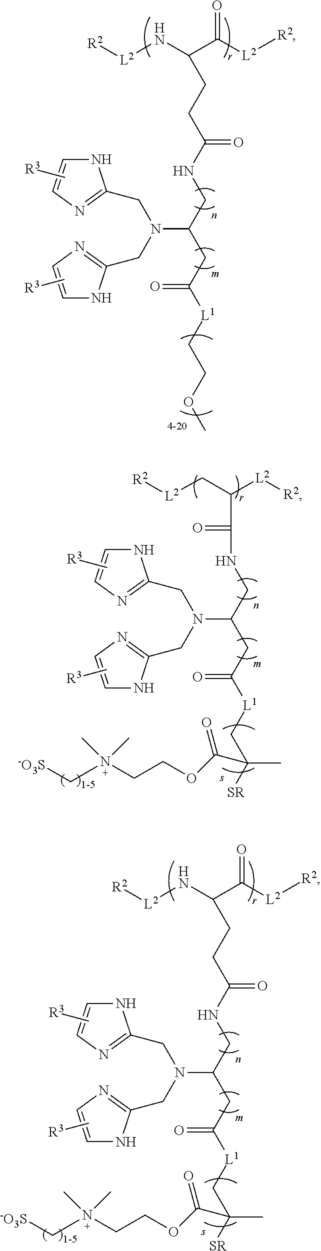 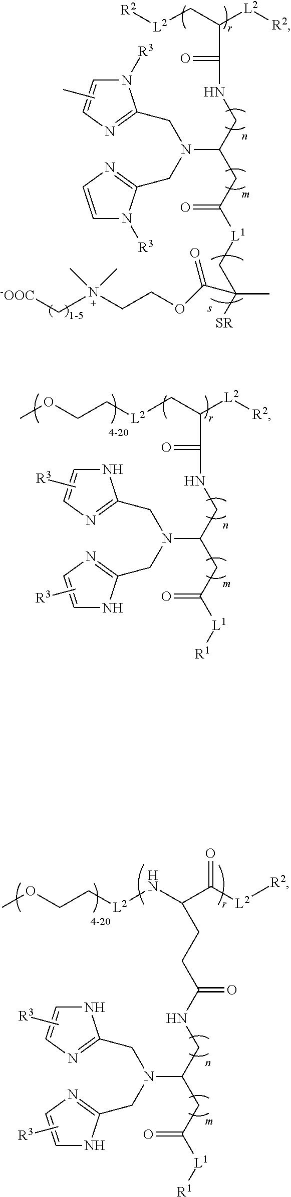 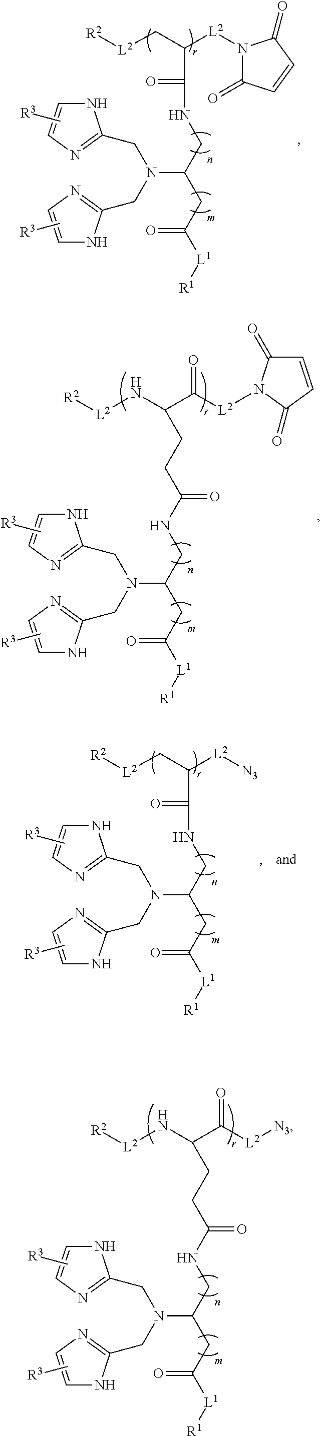
or wherein the compound is selected from 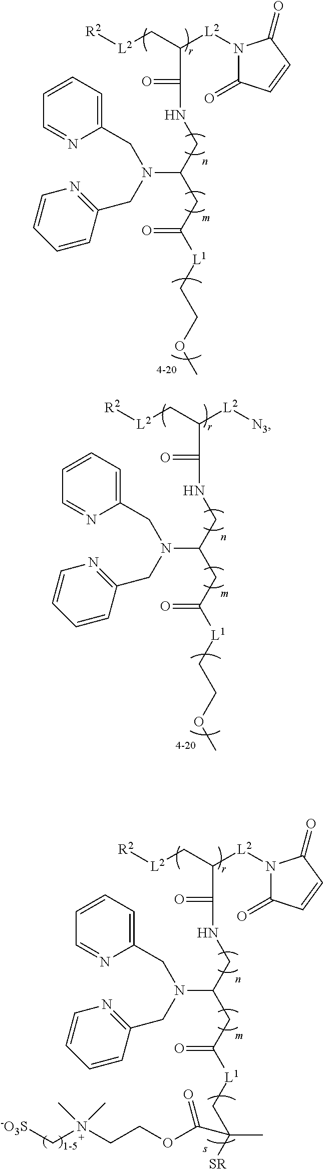 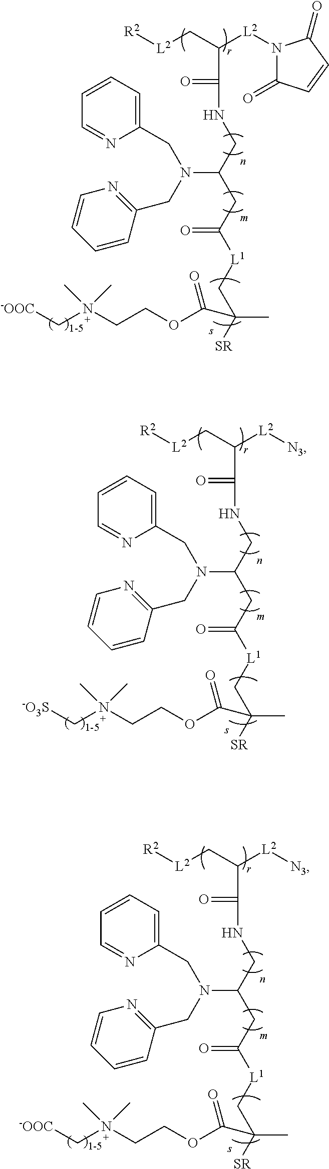 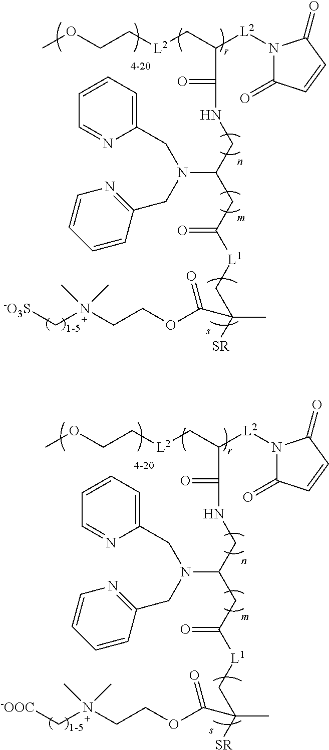 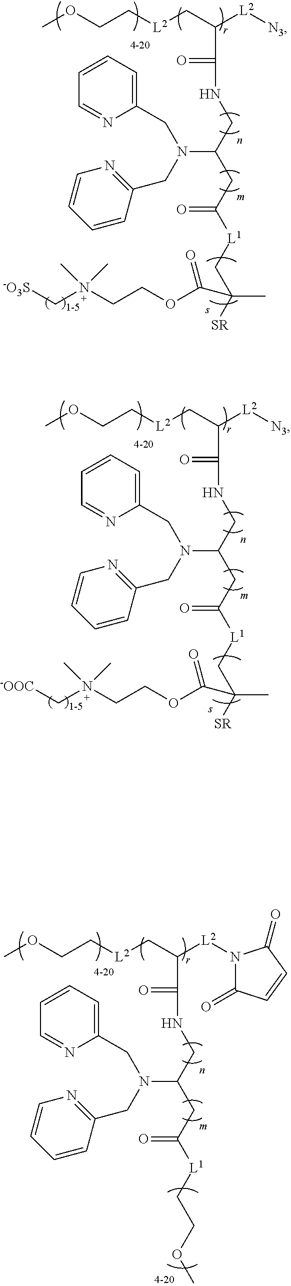 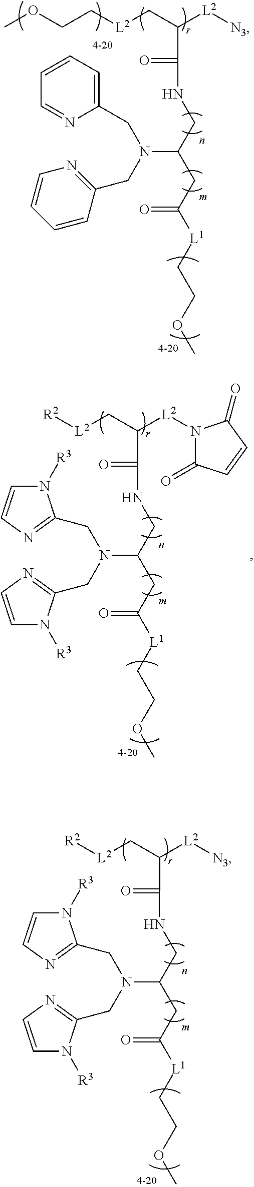 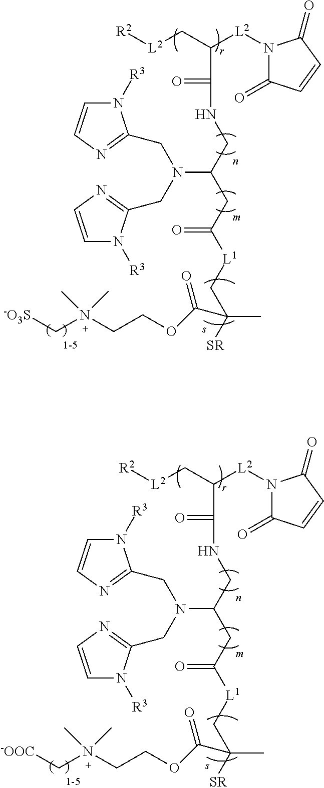 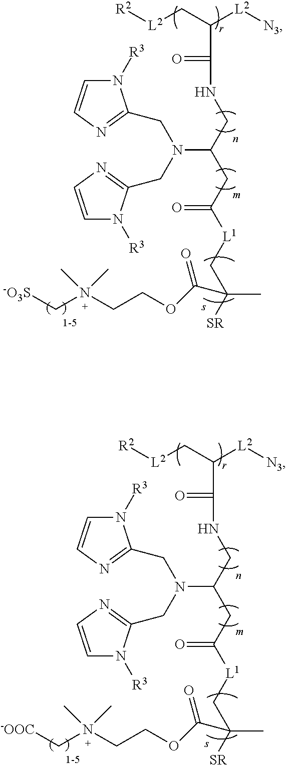 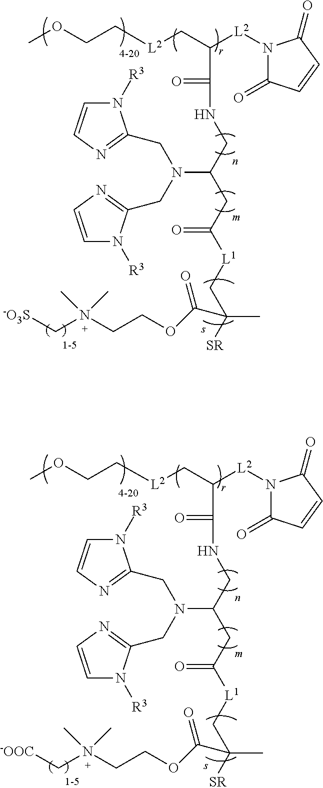 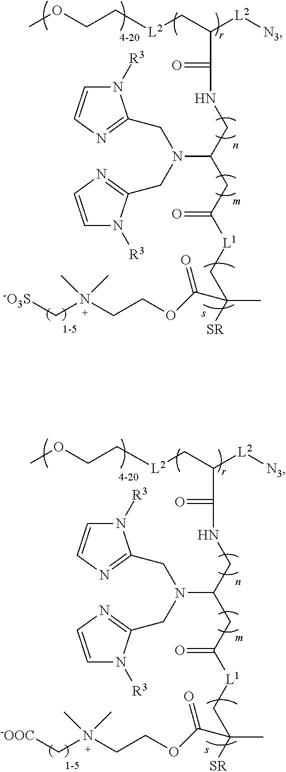 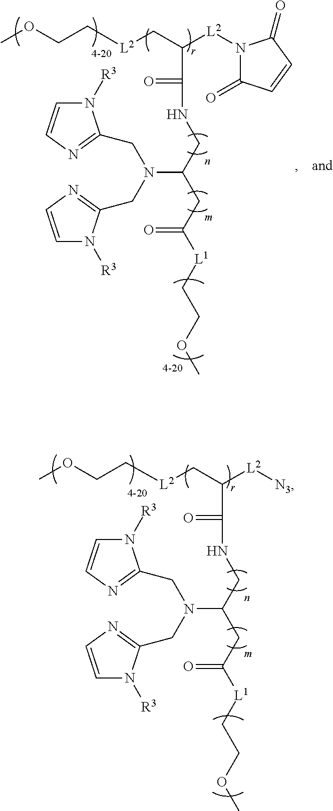
or wherein the compound is selected from
wherein s is about 1 to about 50, about 2 to about 40, about 5 to about 30, about 10 to about 30, about 5 to about 35, or about 20 to about 30, and r is about 3 to about 200, about 6 to about 30, or about 10 to about 25, and wherein R¹, R², L¹, L² and R³ are each as defined in any one of claims 7 to 8.

15. The compound of claim 1, wherein the compound is selected from 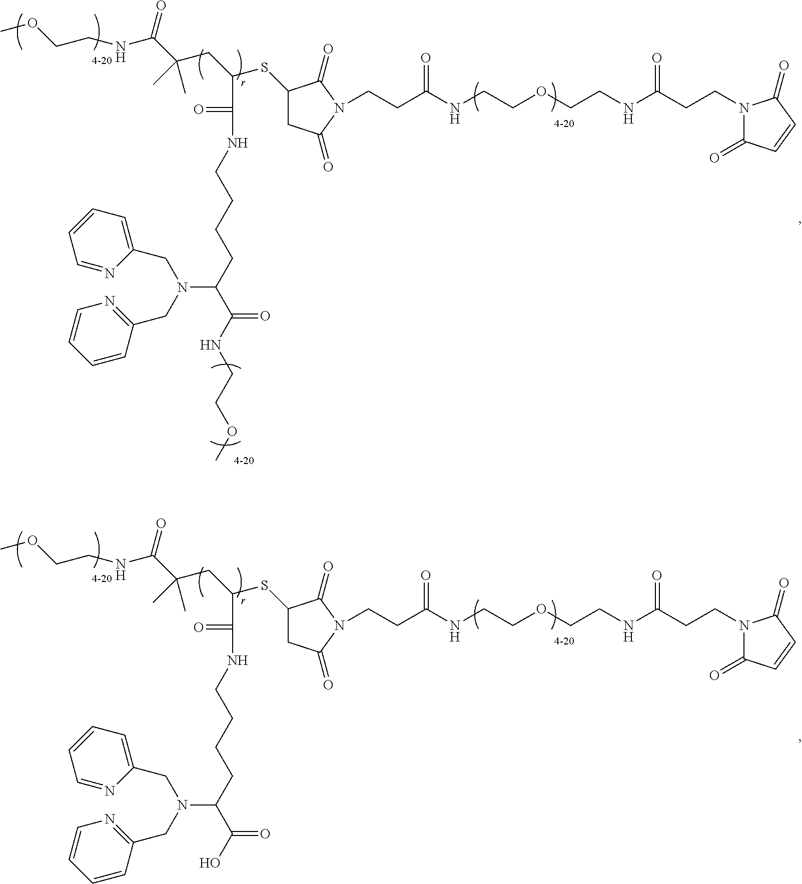 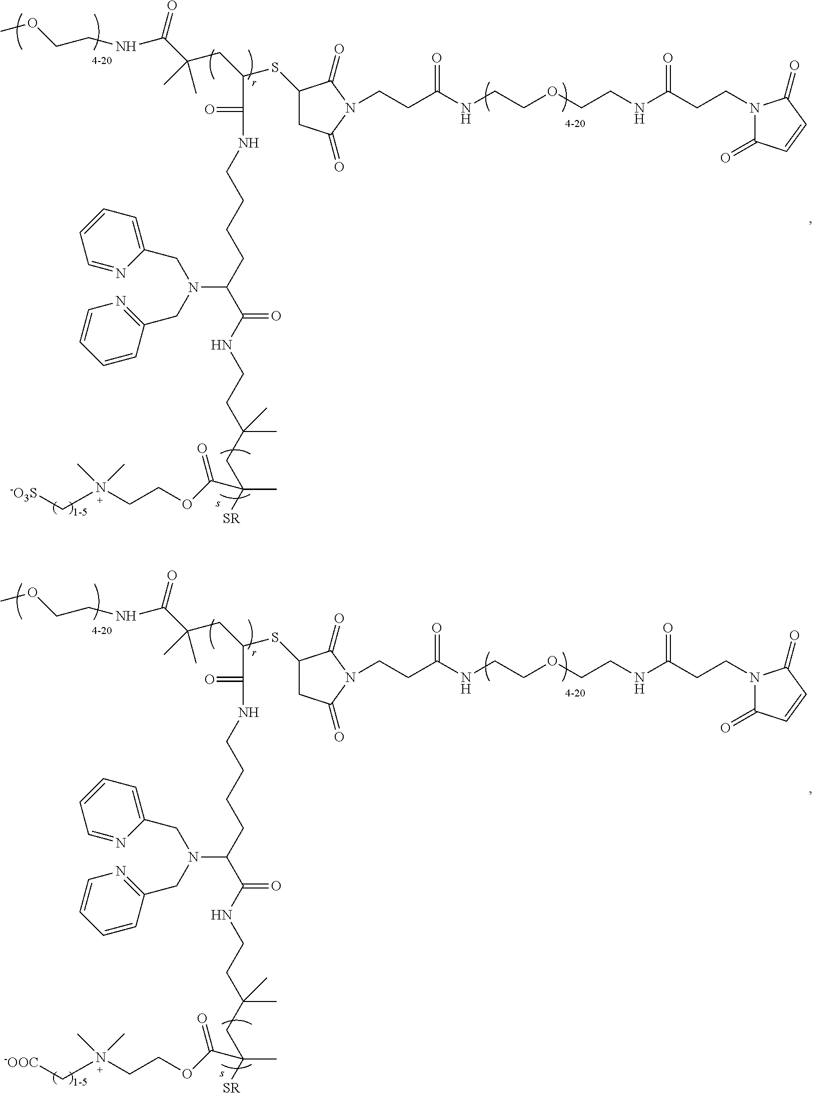 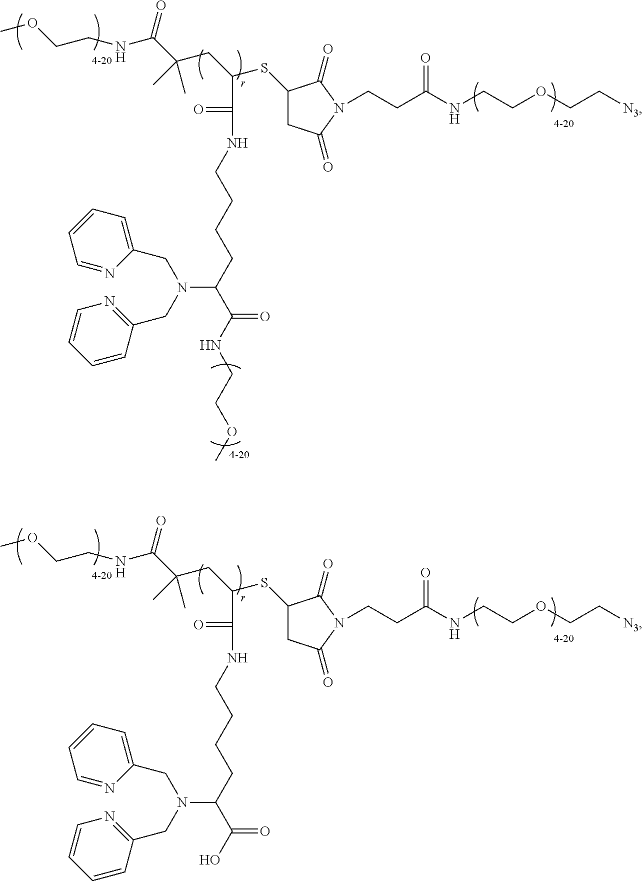 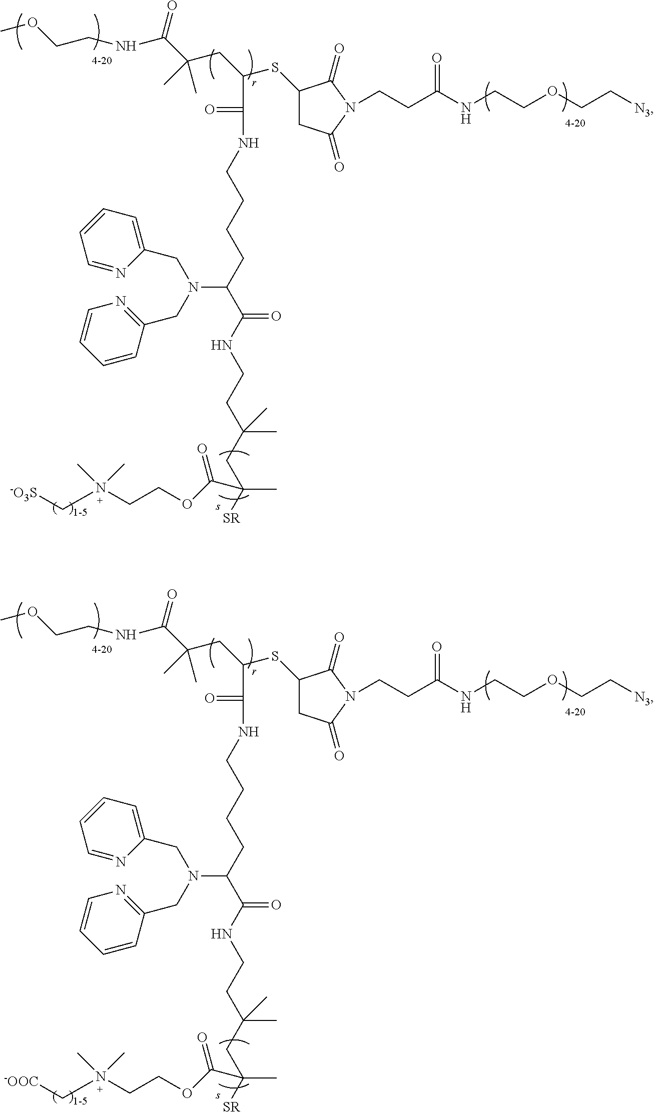 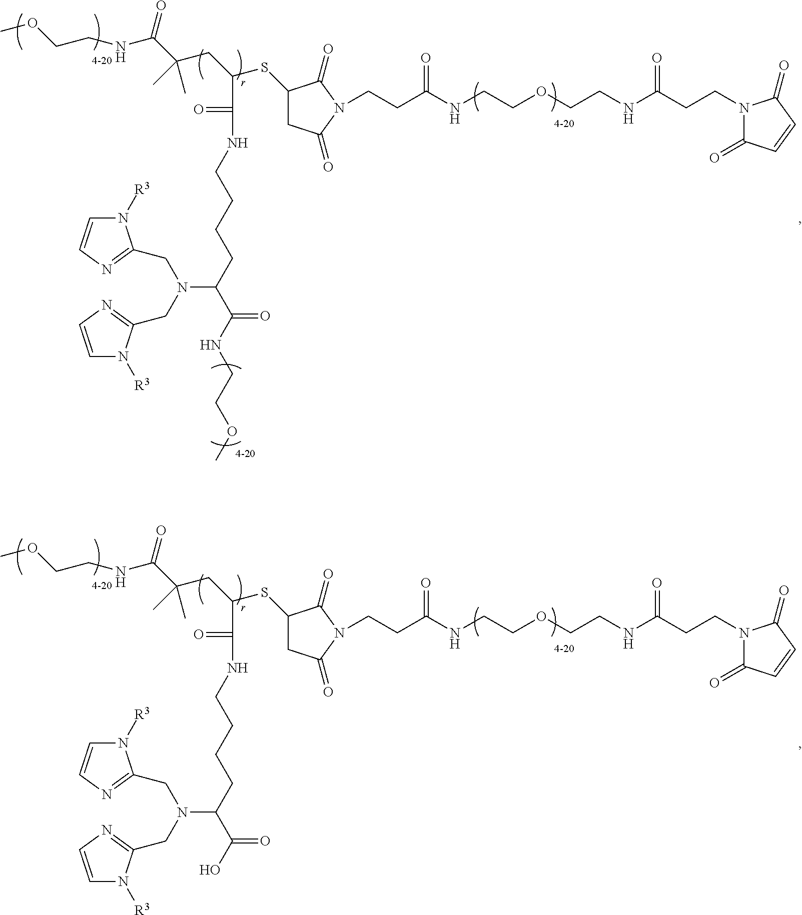 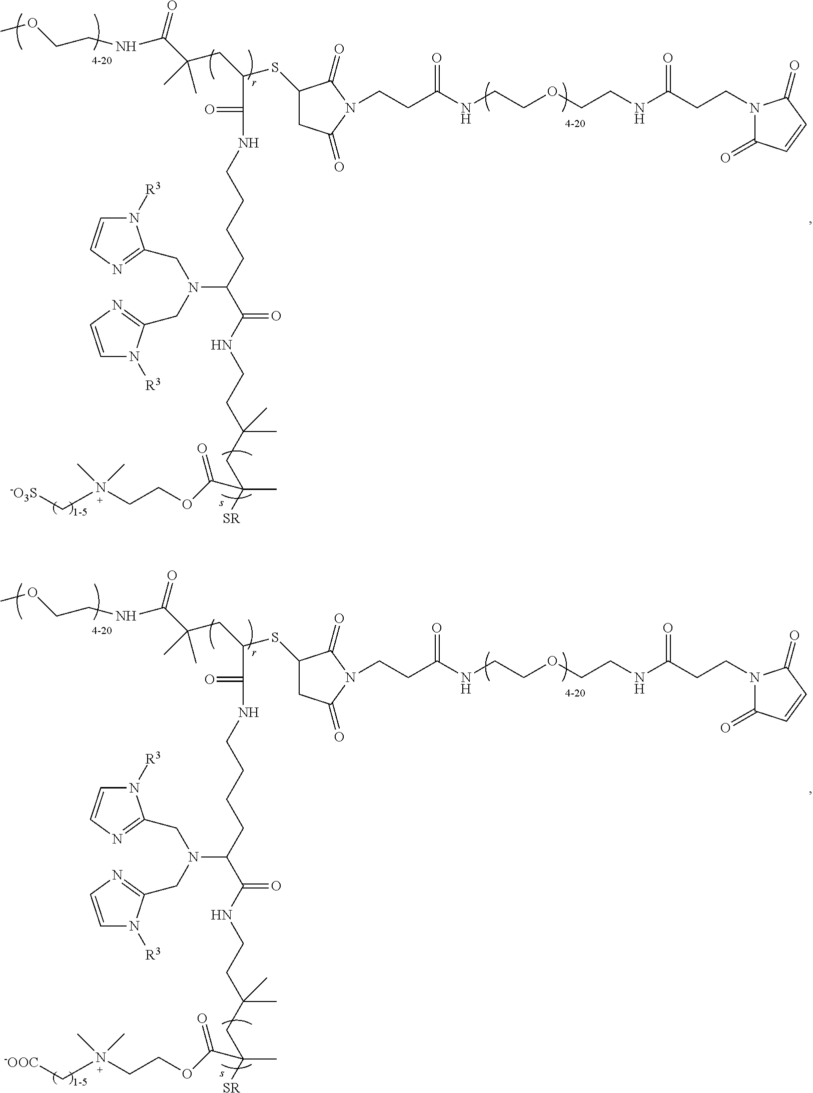 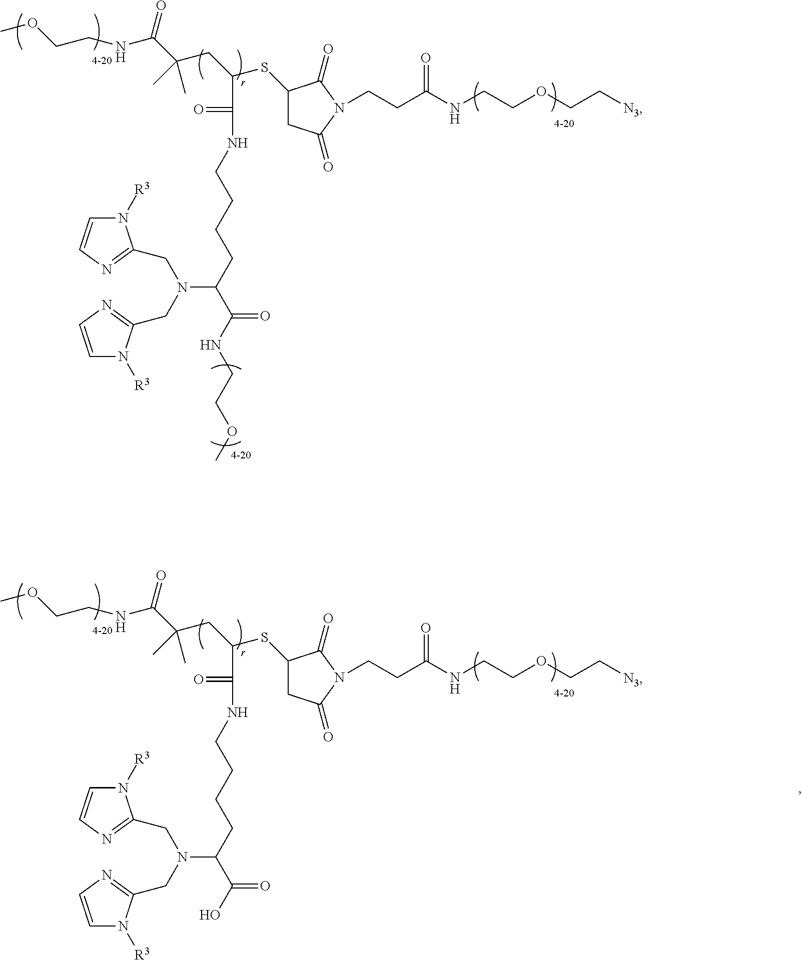 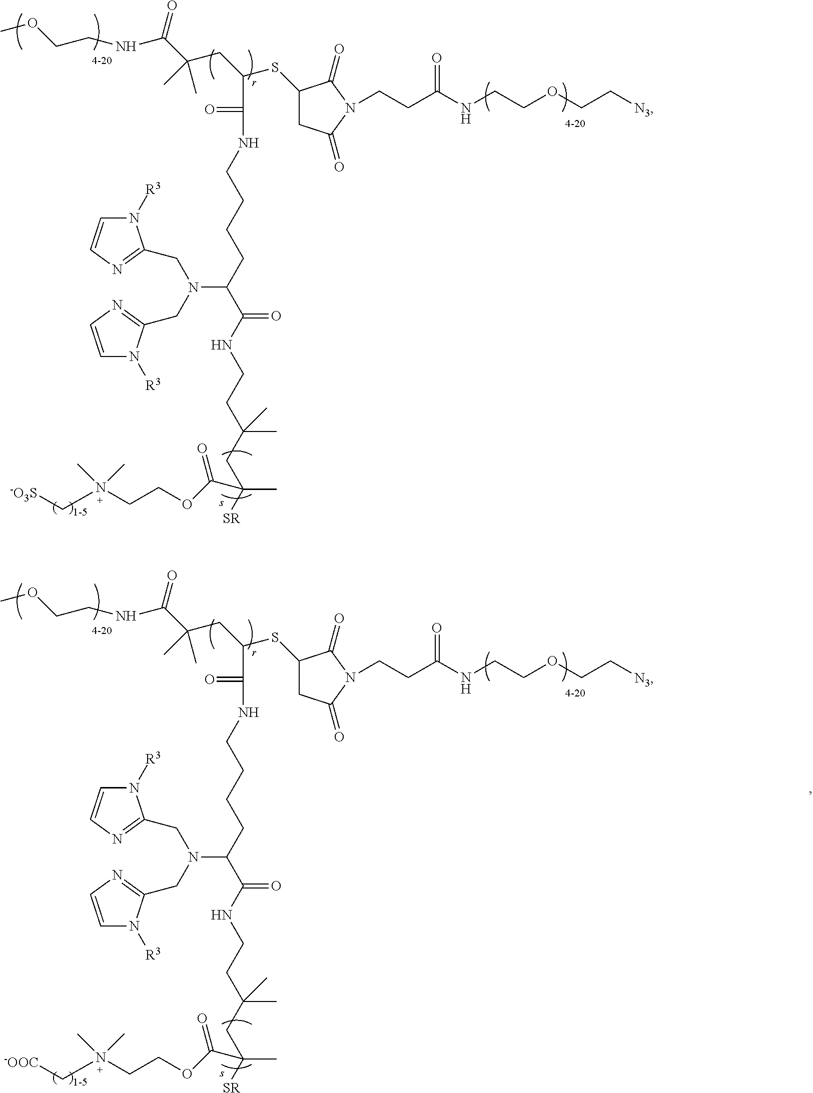 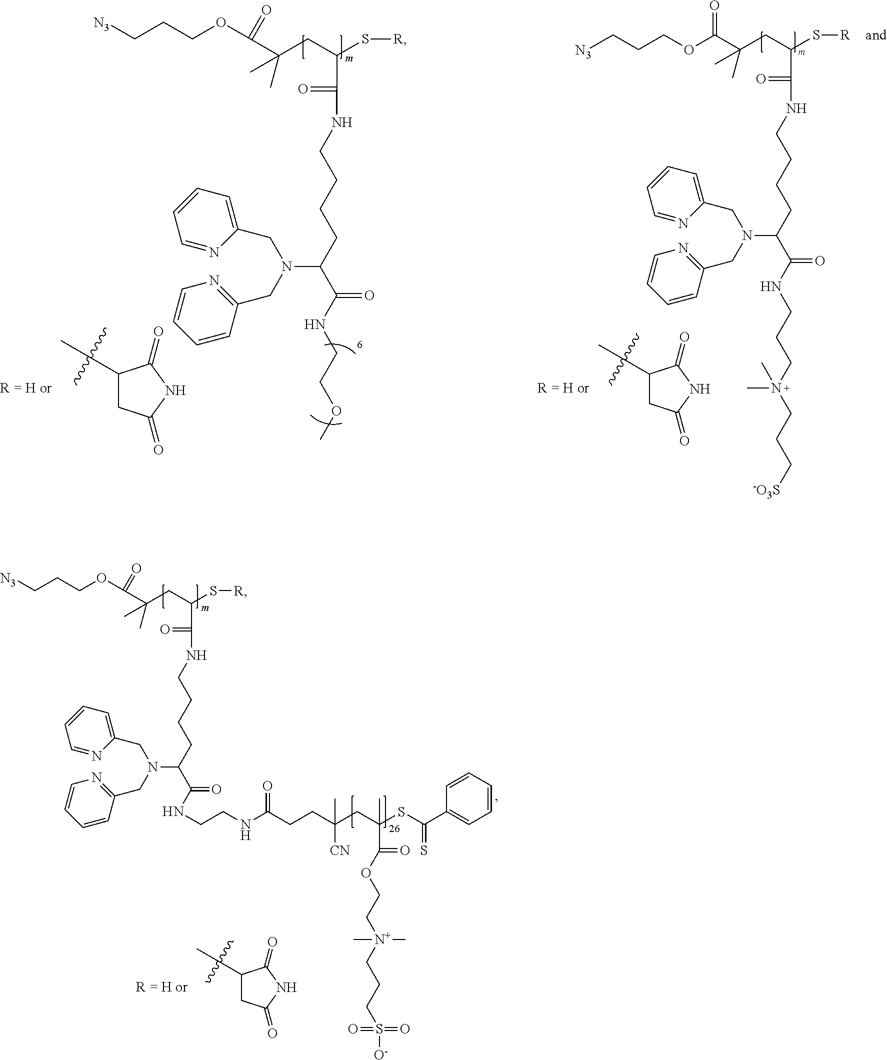
wherein s is about 1 to about 50, about 2 to about 40, about 5 to about 30, about 10 to about 30, about 5 to about 35, or about 20 to about 30, and r is about 3 to about 200, about 6 to about 30, or about 10 to about 25, and wherein R³ is as defined in any one of claims 7 to 8.

16. A compound of Formula I as defined in any one of claims 1 to 15, wherein the compound of Formula I is chelated to one or more metal M, and wherein the compound has a structure of Formula II 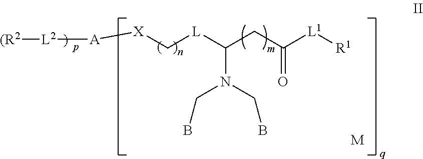
or a derivative or salt thereof.

17. A compound of Formula I as defined in any one of claims 1 to 15 or a compound of Formula II as defined in claim 16 for use in mass cytometry.

18. An element tag comprising a linear or a branched polymer comprising a plurality of chelating groups, wherein each chelating group is capable of binding a soft metal, the soft metal being a single isotope, and wherein at least one chelating group is chelated to a soft metal atom of the soft metal.

19. A kit comprising
an isotopic composition comprising multiple soft metal atoms of a single isotope of a soft metal; and
an element tag comprising a linear or branched polymer comprising a plurality of chelating groups comprising two nitrogen-containing 5-membered or 6-membered heterocycles, wherein each chelating group of the element tag includes at least one soft metal atom of the isotopic composition or is capable of binding at least one soft metal atom of the isotopic composition;
optionally wherein the kit does not comprise any radioactive soft metal.

20. The kit of claim 19, wherein the element tag is functionalised to bind a biomolecule and/or is attached to a biomolecule.

21. The kit of 19 or 20, wherein the isotopic composition is a soft metal solution provided separate from the element tag, and wherein each chelating group is capable of binding at least one soft metal atom of the isotopic composition.

22. The kit of any one of claims 19 to 21 further comprising a biomolecule, optionally an oligonucleotide or an antibody and/or further comprising an additional isotopic composition, wherein the additional isotopic composition comprises multiple additional soft metal atoms of an additional single isotope of a soft metal that is different from the single isotope of the soft metal of the isotopic composition.

23. The kit of any one of claims 19 to 22, wherein each element tag is independently a compound of Formula I as defined in any one of claims 1 to 15 or a compound of Formula II as defined in claim 16.

24. A method comprising:
providing an isotopic composition comprising multiple soft metal atoms of a single isotope of a soft metal;
providing an element tag comprising a linear or branched polymer comprising a plurality of chelating groups each independently comprising two nitrogen-containing 5-membered or 6-membered heterocycles, wherein each chelating group is capable of binding at least one of the soft metal atom of the isotopic composition; and
binding the soft metal atoms of the isotopic composition to the one or more chelating groups of the element tag;
wherein the soft metal atoms are non-radioactive.

25. The method of claim 24, wherein the isotopic composition does not comprise a natural mixture of isotopes, optionally wherein the method further comprises providing an additional isotopic composition wherein the additional isotopic composition comprises multiple additional soft metal atoms of an additional single isotope of a non-radioactive soft metal that is different from the single isotope of the non-radioactive soft metal of the isotopic composition.

26. A method for the analysis of an analyte in a biological sample, comprising:
(i) incubating an element tagged affinity reagent with the analyte, the element tagged affinity reagent comprising an affinity reagent tagged with an element tag, the element tag comprising a linear or branched polymer having multiple chelating groups each independently comprising two nitrogen-containing 5-membered or 6-membered heterocycles, the element tag further comprising multiple soft metal atoms of a single isotope of a soft metal;
wherein:
each chelating group of the element tag includes at least one of the soft metal atoms or is capable of binding at least one of the soft metal atoms,
the soft metal atoms are non-radioactive, and
the affinity reagent specifically binds the analyte,
(ii) separating unbound element tagged affinity reagent from bound element tagged affinity reagent; and
(iii) analyzing the element tag bound to the affinity reagent attached to the analyte by mass spectrometric atomic spectroscopy.

27. The method of claim 26, wherein incubating the element tagged affinity reagent with the analyte comprises:
incubating two or more differential element tagged affinity reagents with two or more analytes, wherein the element tagged affinity reagents specifically bind with the two or more analytes to produce two or more differentially tagged analytes, wherein analyzing the element tag bound to the affinity reagent comprises analyzing the differential element tags bound to the two or more analytes by mass spectrometric atomic spectroscopy.

28. The method of claim 26 or 27, wherein the element tagged affinity reagent is configured to bind to an analyte in a biological sample, and the biological sample comprises cells.

29. The method of any one of claims 26 to 28, wherein the soft metal is selected from Re, Pt, Pd, Nb, Tc, Hg, Ag, Au, Mo, Ru, Rh, Cd, W, Os, or mixtures thereof.

30. The method of any one of claims 24 to 74, wherein the element tag is or comprises a compound of Formula I as defined in any one of claims 1 to 15, or a compound of Formula II as defined in claim 16.
